SEQUENCE LISTING (28 sequences)

SEQ ID NO: 1 - protein, 575 aa, Phaeodactylum tricornutum
MRPYPTIALISVFLSAATRISATSSHQASALPVKKGTHVPDSPKLSKLYIMAKTKSVSSSFDPPRGGSTVAPTTPLATGGALRKVRQAVFPIYGNQEVTKFLLIGSIKFFIILALTLTRDTKDTLIVTQCGAEAIAFLKIYGVLPAATAFIALYSKMSNAMGKKMLFYSTCIPFFTFFGLFDVFIYPNAERLHPSLEAVQAILPGGAASGGMAVLAKIATHWTSALFYVMAEIYSSVSVGLLFWQFANDVVNVDQAKRFYPLFAQMSGLAPVLAGQYVVRFASKAVNFEASMHRLTAAVTFAGIMICIFYQLSSSYVERTESAKPAADNEQSIKPKKKKPKMSMVESGKFLASSQYLRLIAMLVLGYGLSINFTEIMWKSLVKKQYPDPLDYQRFMGNFSSAVGLSTCIVIFFGVHVIRLLGWKVGALATPGIMAILALPFFACILLGLDSPARLEIAVIFGTIQSLLSKTSKYALFDPTTQMAYIPLDDESKVKGKAAIDVLGSRIGKSGGSLIQQGLVFVFGNIINAAPVVGVVYYSVLVAWMSAAGRLSGLFQAQTEMDKADKMEAKTNKEK

SEQ ID NO: 2 - DNA, 40 bp, Artificial Sequence
  Description of Artificial Sequence Synthetic oligonucleotide
atgggtctcacacaaactcgagtacaactttaactcacac

SEQ ID NO: 3 - DNA, 33 bp, Artificial Sequence
  Description of Artificial Sequence Synthetic oligonucleotide
atgggtctcgattccattcttttgtttgtctgc

SEQ ID NO: 4 - DNA, 31 bp, Artificial Sequence
  Description of Artificial Sequence Synthetic oligonucleotide
cataatggtctcgctgctgcccgataaccac

SEQ ID NO: 5 - DNA, 42 bp, Artificial Sequence
  Description of Artificial Sequence Synthetic oligonucleotide
tgatattggtctcggtctttcgataaaacactctgagtagag

SEQ ID NO: 6 - DNA, 35 bp, Artificial Sequence
  Description of Artificial Sequence Synthetic oligonucleotide
atgggtctcgaaacctgatcatgtagatcgaacgg

SEQ ID NO: 7 - DNA, 28 bp, Artificial Sequence
  Description of Artificial Sequence Synthetic oligonucleotide
atgggtctcatctaacccggctgaacgg

SEQ ID NO: 8 - DNA, 32 bp, Artificial Sequence
  Description of Artificial Sequence Synthetic oligonucleotide
atgggtctccggtagttcagcagggcagaacg

SEQ ID NO: 9 - DNA, 34 bp, Artificial Sequence
  Description of Artificial Sequence Synthetic oligonucleotide
atgggtctcggaggggatttgaacccctgccatg

SEQ ID NO: 10 - DNA, 34 bp, Artificial Sequence
  Description of Artificial Sequence Synthetic oligonucleotide
atattcggtctcgtcagcagaatacgccgattgg

SEQ ID NO: 11 - DNA, 33 bp, Artificial Sequence
  Description of Artificial Sequence Synthetic oligonucleotide
acgcgttggtctcggttatcgggcagcagcacc

SEQ ID NO: 12 - DNA, 29 bp, Artificial Sequence
  Description of Artificial Sequence Synthetic oligonucleotide
attggtctcggccgagcggttgaaggcac

SEQ ID NO: 13 - DNA, 29 bp, Artificial Sequence
  Description of Artificial Sequence Synthetic oligonucleotide
attggtctctctggaaccctttcgggtcg

SEQ ID NO: 14 - DNA, 24 bp, Artificial Sequence
  Description of Artificial Sequence Synthetic oligonucleotide
ctcgagtacaactttaactcacac

SEQ ID NO: 15 - DNA, 24 bp, Artificial Sequence
  Description of Artificial Sequence Synthetic oligonucleotide
gattccattcttttgtttgtctgc

SEQ ID NO: 16 - DNA, 19 bp, Artificial Sequence
  Description of Artificial Sequence Synthetic oligonucleotide
gctgctgcccgataaccac

SEQ ID NO: 17 - DNA, 29 bp, Artificial Sequence
  Description of Artificial Sequence Synthetic oligonucleotide
ggtctttcgataaaacactctgagtagag

SEQ ID NO: 18 - DNA, 26 bp, Artificial Sequence
  Description of Artificial Sequence Synthetic oligonucleotide
gaaacctgatcatgtagatcgaacgg

SEQ ID NO: 19 - DNA, 19 bp, Artificial Sequence
  Description of Artificial Sequence Synthetic oligonucleotide
atctaacccggctgaacgg

SEQ ID NO: 20 - DNA, 23 bp, Artificial Sequence
  Description of Artificial Sequence Synthetic oligonucleotide
ccgctgccactaggaagcttatg

SEQ ID NO: 21 - DNA, 27 bp, Artificial Sequence
  Description of Artificial Sequence Synthetic oligonucleotide
cctctagaaaatcattccggaagtgtg

SEQ ID NO: 22 - DNA, 56 bp, Artificial Sequence
  Description of Artificial Sequence Synthetic oligonucleotide
  modified_base (30)..(30): An unnatural nucleotide
  See specification as filed for detailed description of substitutions and preferred embodiments
ctctggaaccctttcgggtcgccggtttgntagaccggtgccttcaaccgctcggc

SEQ ID NO: 23 - DNA, 63 bp, Artificial Sequence
  Description of Artificial Sequence Synthetic oligonucleotide
  modified_base (31)..(33): a, c, t, or g
  See specification as filed for detailed description of substitutions and preferred embodiments
ctcgagtacaactttaactcacacaatgtannnatcacggcagacaaacaaaagaatggaatc

SEQ ID NO: 24 - DNA, 49 bp, Artificial Sequence
  Description of Artificial Sequence Synthetic oligonucleotide
  modified_base (23)..(23): An unnatural nucleotide
  See specification as filed for detailed description of substitutions and preferred embodiments
cagcagaatacgccgattggcgntggcccggtgctgctgcccgataacc

SEQ ID NO: 25 - DNA, 53 bp, Artificial Sequence
  Description of Artificial Sequence Synthetic oligonucleotide
  modified_base (21)..(21): An unnatural nucleotide
  See specification as filed for detailed description of substitutions and preferred embodiments
gctgctgcccgataaccacancctctctactcagagtgttttatcgaaagacc

SEQ ID NO: 26 - DNA, 43 bp, Artificial Sequence
  Description of Artificial Sequence Synthetic oligonucleotide
  modified_base (19)..(19): An unnatural nucleotide
  See specification as filed for detailed description of substitutions and preferred embodiments
gctgcccgataaccacagnttgtctactcagagtgttttatcg

SEQ ID NO: 27 - DNA, 52 bp, Artificial Sequence
  Description of Artificial Sequence Synthetic oligonucleotide
  modified_base (25)..(27): a, c, t, or g
  See specification as filed for detailed description of substitutions and preferred embodiments
gaatctaacccggctgaacggattnnnagtccgttcgatctacatgatcagg

SEQ ID NO: 28 - DNA, 52 bp, Artificial Sequence
  Description of Artificial Sequence Synthetic oligonucleotide
  modified_base (27)..(27): An unnatural nucleotide
  See specification as filed for detailed description of substitutions and preferred embodiments
gatttgaacccctgccatgcggattancagtccgccgttctgccctgctgaa

(19) United States
(12) Patent Application Publication
(10) Pub. No.: US 2022/0243244 A1
(43) Pub. Date: Aug. 4, 2022

(54) COMPOSITIONS AND METHODS FOR IN VIVO SYNTHESIS OF UNNATURAL POLYPEPTIDES

(71) Applicant: The Scripps Research Institute, La Jola, CA (US)

(72) Inventors: Floyd E. ROMESBERG, La Jolla, CA (US); Emil C. FISCHER, La Jolla, CA (US); Koji HASHIMOTO, La Jolla, CA (US); Aaron W. FELDMAN, La Jolla, CA (US); Vivian T. DIEN, La Jolla, CA (US); Yorke ZHANG, La Jolla, CA (US)

(21) Appl. No.: 17/716,848

(22) Filed: Apr. 8, 2022

Related U.S. Application Data
(63) Continuation of application No. PCT/US2020/054947, filed on Oct. 9, 2020.
(60) Provisional application No. 62/988,882, filed on Mar. 12, 2020.
(60) Provisional application No. 62/913,664, filed on Oct. 10, 2019.

(51) Int. Cl. C12P 21/02

(52) U.S. Cl. CPC C12P 21/02

(57) ABSTRACT

Disclosed herein are compositions, methods, and kits for a cell incorporating unnatural amino acids into an unnatural polypeptide. Also disclosed herein are compositions, methods, and kits for increasing activity and yield of the unnatural polypeptide synthesized by the cell.

### CROSS-REFERENCE TO RELATED APPLICATIONS

This application is a continuation of International Application No. PCT/US2020/054947, filed Oct. 9, 2020, which claims priority to U.S. Provisional Application No. 62/913,664, filed on Oct. 10, 2019, and U.S. Provisional Application No. 62/988,882, filed on Mar. 12, 2020, each of which is incorporated by reference herein in its entirety.

### STATEMENT AS TO FEDERALLY SPONSORED RESEARCH

This invention was made with government support under Grant No. GM118178 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 6, 2010 is named “36271-809_301_SL.txt” and is 21 kilobytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

To the extent publications and patents or patent applications incorporated by reference contradict the disclosure contained in the specification, the specification is intended to supersede and/or take precedence over any such contradictory material.

### BACKGROUND

The natural genetic code consists of 64 codons made possible by four letters of the genetic alphabet. Three codons are used as stop codons, leaving 61 sense codons that are recognized by a transfer RNA (tRNA) charged by a cognate amino acyl tRNA synthetase (also referred to herein simply as a tRNA synthetase) with one of the 20 proteogenic amino acids. While the canonical amino acids have enabled the remarkable diversity of living organisms, there are many chemical functionalities and associated reactivities that they do not provide. The ability to expand the genetic code to include unnatural or non-canonical amino acids (ncAAs) likely bestows the protein with a desired function or activity and dramatically facilitates many known and emerging applications of proteins such as therapeutic development. Current methods of synthesizing unnatural proteins or unnatural polypeptides containing unnatural amino acids have limitations. Notably, most methods only enable introduction of a single unnatural amino acid or a few copies of one species of unnatural amino acid into an unnatural polypeptide. Also, the unnatural polypeptide synthesized by the methods currently available often possesses reduced enzymatic activity, solubility, or yield.

One alternative solution to address these limitations is to synthesize the unnatural polypeptides with a cell-free or in vitro expression system. However, such expression system is inadequate in providing a post-translation modification environment where the redox properties of the unnatural polypeptide and other post-translational modifications of the synthesized unnatural polypeptide are fully realized. Therefore, there remains a need for compositions and methods for in vivo synthesis of unnatural polypeptides containing unnatural amino acids.

### SUMMARY

Described herein are compositions, methods, cells (both non-engineered and engineered), semi-synthetic organisms (SSOs), reagents, genetic material, plasmids, and kits for in vivo synthesis of unnatural polypeptides or unnatural proteins, where each unnatural polypeptide or unnatural protein comprises two or more unnatural amino acids that are decoded by the cells.

Described herein are in vivo methods of synthesizing an unnatural polypeptide comprising: providing at least one unnatural deoxyribonucleic acid (DNA) molecule comprising at least four unnatural base pairs; transcribing the at least one unnatural DNA molecule to afford a messenger ribonucleic acid (mRNA) molecule comprising at least two unnatural codons; transcribing the at least one unnatural DNA molecule to afford at least two transfer RNA (tRNA) molecules each comprising at least one unnatural anticodon, wherein the at least two unnatural base pairs in the corresponding DNA are in sequence contexts such that the unnatural codons of the mRNA molecule are complementary to the unnatural anticodon of each of the tRNA molecules; and synthesizing the unnatural polypeptide by translating the unnatural mRNA molecule utilizing the at least two unnatural tRNA molecules, wherein each unnatural anticodon directs the site-specific incorporation of an unnatural amino acid into the unnatural polypeptide. In some embodiments, the at least two unnatural base pairs comprise base pairs selected from dCNMO-dTPT3, dNaM-dTPT3, dCNMO-dTAT1, or dNaM-dTAT1.

In some embodiments, a method of synthesizing an unnatural polypeptide is provided, comprising: providing at least one unnatural deoxyribonucleic acid (DNA) molecule comprising at least four unnatural base pairs, wherein the at least one unnatural DNA molecule encodes (i) a messenger ribonucleic acid (mRNA) molecule comprising at least first and second unnatural codons and (ii) at least first and second transfer RNA (tRNA) molecules, the first tRNA molecule comprising a first unnatural anticodon and the second tRNA molecule comprising a second unnatural anticodon, and the at least four unnatural base pairs in the at least one DNA molecule are in sequence contexts such that the first and second unnatural codons of the mRNA molecule are complementary to the first and second unnatural anticodons, respectively; transcribing the at least one unnatural DNA molecule to afford the mRNA; transcribing the at least one unnatural DNA molecule to afford the at least first and second tRNA molecules; and synthesizing the unnatural polypeptide by translating the unnatural mRNA molecule utilizing the at least first and second unnatural tRNA molecules, wherein each of the at least first and second unnatural anticodons direct site-specific incorporation of an unnatural amino acid into the unnatural polypeptide.

In some embodiments, the methods comprise the at least two unnatural codons each comprising a first unnatural nucleotide positioned at a first position, a second position, or a third position of the codon, optionally wherein the first unnatural nucleotide is positioned at a second position or a third position of the codon. In some instances, the methods comprise at least two unnatural codons each comprising a nucleic acid sequence NNX or NXN, and the unnatural anticodon comprising a nucleic acid sequence XNN, YNN, NXN, or NYN, to form the unnatural codon-anticodon pair comprising NNX-XNN, NNX-YNN, or NXN-NYN, wherein N is any natural nucleotide, X is a first unnatural nucleotide, and Y is a second unnatural nucleotide different from the first unnatural nucleotide, with X-Y forming the unnatural base pair (UBP) in DNA.

In some embodiments, UBPs are formed between the codon sequence of the mRNA and the anticodon sequence of the tRNA to facilitate translation of the mRNA into an unnatural polypeptide. Codon-anticodon UBPs comprise, in some instances, a codon sequence comprising three contiguous nucleic acids read 5′ to 3′ of the mRNA (e.g., UUX), and an anticodon sequence comprising three contiguous nucleic acids ready 5′ to 3′ of the tRNA (e.g., YAA or XAA). In some embodiments, when the mRNA codon is UUX, the tRNA anticodon is YAA or XAA. In some embodiments, when the mRNA codon is UGX, the tRNA anticodon is YCA or XCA. In some embodiments, when the mRNA codon is CGX, the tRNA anticodon is YCG or XCG. In some embodiments, when the mRNA codon is AGX, the tRNA anticodon is YCU or XCU. In some embodiments, when the mRNA codon is GAX, the tRNA anticodon is YUC or XUC. In some embodiments, when the mRNA codon is CAX, the tRNA anticodon is YUG or XUG. In some embodiments, when the mRNA codon is GXU, the tRNA anticodon is AYC. In some embodiments, when the mRNA codon is CXU, the tRNA anticodon is AYG. In some embodiments, when the mRNA codon is GXG, the tRNA anticodon is CYC. In some embodiments, when the mRNA codon is AXG, the tRNA anticodon is CYU. In some embodiments, when the mRNA codon is GXC, the tRNA anticodon is GYC. In some embodiments, when the mRNA codon is AXC, the tRNA anticodon is GYU. In some embodiments, when the mRNA codon is GXA, the tRNA anticodon is UYC. In some embodiments, when the mRNA codon is CXC, the tRNA anticodon is GYG. In some embodiments, when the mRNA codon is UXC, the tRNA anticodon is GYA. In some embodiments, when the mRNA codon is AUX, the tRNA anticodon is YAU or XAU. In some embodiments, when the mRNA codon is CUX, the tRNA anticodon is XAG or YAG. In some embodiments, when the mRNA codon is UUX, the tRNA anticodon is XAA or YAA. In some embodiments, when the mRNA codon is GUX, the tRNA anticodon is XAC or YAC. In some embodiments, when the mRNA codon is UAX, the tRNA anticodon is XUA or YUA. In some embodiments, when the mRNA codon is GGX, the tRNA anticodon is XCC or YCC.

In some embodiments, the at least one unnatural DNA molecule is transcribed into messenger RNA (mRNA) comprising the unnatural bases described herein (e.g., d5SICS, dNaM, dTPT3, dMTMO, dCNMO, dTAT1). Exemplary mRNA codons are coded by exemplary regions of the unnatural DNA comprising three contiguous deoxyribonucleotides (NNN) comprising TTX, TGX, CGX, AGX, GAX, CAX, GXT, CXT, GXG, AXG, GXC, AXC, GXA, CXC, TXC, ATX, CTX, TTX, GTX, TAX, or GGX, where X is the unnatural base attached to a 2′ deoxyribosyl moiety. The exemplary mRNA codons resulting from transcription of the exemplary unnatural DNA comprise three contiguous ribonucleotides (NNN) comprising UUX, UGX, CGX, AGX, GAX, CAX, GXU, CXU, GXG, AXG, GXC, AXC, GXA, CXC, UXC, AUX, CUX, UUX, GUX, UAX, or GGX, respectively, wherein X is the unnatural base attached to a ribosyl moiety. In some embodiments, the unnatural base is in a first position in the codon sequence (X-N-N). In some embodiments, the unnatural base is in a second (or middle) position in the codon sequence (N-X-N). In some embodiments, the unnatural base is in a third (last) position in the codon sequence (N-N-X).

In some embodiments, the methods comprise the codon comprising at least one G and the anticodon comprising at least one C. In some instances, the methods comprise X and Y, where X and Y are independently selected from the group consisting of: (i) 2-thiouracil, 2′-deoxyuridine, 4-thio-uracil, uracil-5-yl, hypoxanthin-9-yl (I), 5-halouracil; 5-propynyl-uracil, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil; (ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1,4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one); (iii) 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine; (iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and (v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone. In some embodiments, X and Y are independently selected from the group consisting of:

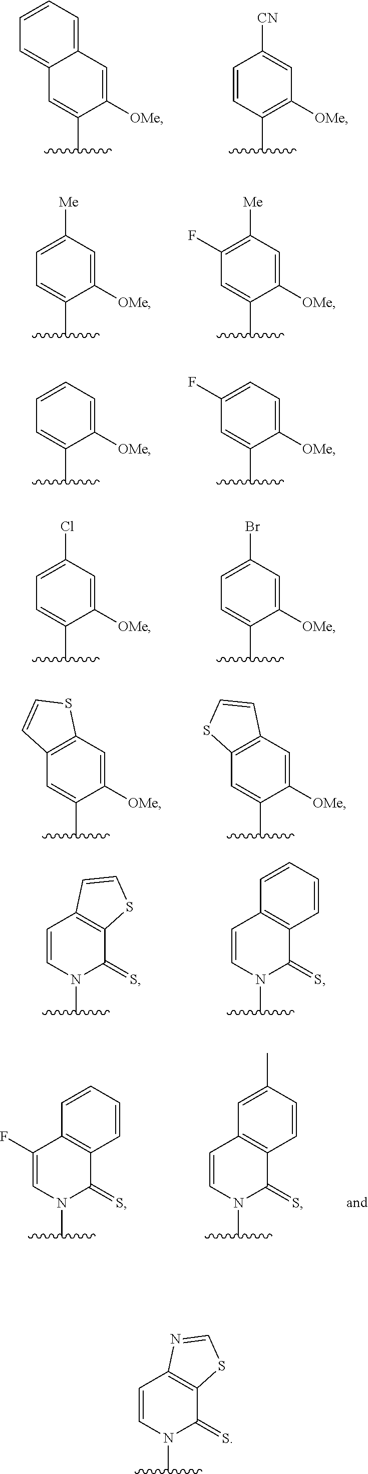

In some cases, the X is

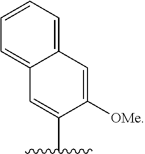 [SMILES: *c1cc2ccccc2cc1OC]

In some embodiments, the Y is

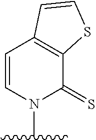 [SMILES: *n1ccc2ccsc2c1=S]

In some embodiments, the methods described herein comprise unnatural codon-anticodon pair NNX-XNN, where NNX-XNN is selected from the group consisting of UUX-XAA, UGX-XCA, CGX-XCG, AGX-XCU, GAX-XUC, CAX-XUG, AUX-XAU, CUX-XAG, GUX-XAC, UAX-XUA, and GGX-XCC. In some embodiments, the methods described herein comprise unnatural codon-anticodon pair NNX-YNN, where NNX-YNN is selected from the group consisting of UUX-YAA, UGX-YCA, CGX-YCG, AGX-YCU, GAX-YUC, CAX-YUG, AUX-YAU, CUX-YAG, GUX-YAC, UAX-YUA, and GGX-YCC. In some instances, the methods described herein comprise unnatural codon-anticodon pair NXN-NYN, where NXN-NYN is selected from the group consisting of GXU-AYC, CXU-AYG, GXG-CYC, AXG-CYU, GXC-GYC, AXC-GYU, GXA-UYC, CXC-GYG, and UXC-GYA. In some embodiments, the methods described herein comprise at least two unnatural tRNA molecules each comprising a different unnatural anticodon. In some instances, the at least two unnatural tRNA molecules comprise a pyrrolysyl tRNA from the Methanosarcina genus and the tyrosyl tRNA from Methanocaldococcus jannaschii, or derivatives thereof. In some embodiments, the methods comprise charging the at least two unnatural tRNA molecules by an amino-acyl tRNA synthetase. In some instances, the tRNA synthetase is selected from a group consisting of chimeric PylRS (chPylRS) and M. jannaschii AzFRS (MjpAzFRS). In some embodiments, the methods as described herein comprise charging the at least two unnatural tRNA molecules by at least two different tRNA synthetases. In some cases, the at least two different tRNA synthetases comprise chimeric PylRS (chPylRS) and M. jannaschii AzFRS (MjpAzFRS).

Described herein, in some embodiments, are methods of in vivo synthesis of unnatural polypeptides. In some embodiments, the unnatural polypeptide comprises two, three, or more unnatural amino acids. In some cases, the unnatural polypeptide comprises at least two unnatural amino acids that are the same. In some embodiments, the unnatural polypeptide comprises at least two different unnatural amino acids. In some instances, the unnatural amino acid comprises:

a lysine analogue; an aromatic side chain; an azido group; an alkyne group; or an aldehyde or ketone group. In some instances, the unnatural amino acid does not comprise an aromatic side chain. In some embodiments, the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

In some embodiments, the methods of in vivo synthesis of unnatural polypeptides as described herein comprise at least one unnatural DNA molecule in the form of a plasmid. In some cases, the at least one unnatural DNA molecule is integrated into the genome of a cell. In some embodiments, the at least one unnatural DNA molecule encodes the unnatural polypeptide. In some embodiments, the methods described herein comprise in vivo replication and transcription of the unnatural DNA molecule and in vivo translation of the transcribed mRNA molecule in a cellular organism. In some embodiments, the cellular organism is a microorganism. In some embodiments, the cellular organism is a prokaryote. In some embodiments, the cellular organism is a bacterium. In some instances, the cellular organism is a gram-positive bacterium. In some embodiments, the cellular organism is a gram-negative bacterium. In some instances, the cellular organism is Escherichia coli. In some embodiments, the cellular organism comprises a nucleoside triphosphate transporter. In some cases, the nucleoside triphosphate transporter comprises the amino acid sequence of PtNTT2. In some embodiments, the nucleoside triphosphate transporter comprises a truncated amino acid sequence of PtNTT2. In some alternatives, the truncated amino acid sequence of PtNTT2 is at least 80% identical to aPtNTT2 encoded by SEQ ID NO.1. In some embodiments, the cellular organism comprises the at least one unnatural DNA molecule. In some embodiments, the at least one unnatural DNA molecule comprises at least one plasmid. In some embodiments, the at least one unnatural DNA molecule is integrated into genome of the cell. In some cases, the at least one unnatural DNA molecule encodes the unnatural polypeptide. In some instances, the methods described in this instant disclosure can be an in vitro method comprising synthesizing the unnatural polypeptide with a cell-free system.

Described herein, in some embodiments, are methods for in vivo synthesis of unnatural polypeptides, where the unnatural polypeptides comprise an unnatural sugar moiety. In some embodiments, the unnatural base pairs comprise at least one unnatural nucleotide comprising an unnatural sugar moiety. In some embodiments, the unnatural sugar moiety is selected from the group consisting of: OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —O(CH₂)ₙON[(CH₂)ₙCH₃)]₂, wherein n and m are from 1 to about 10; and/or a modification at the 5′ position: 5′-vinyl, 5′-methyl (R or S); a modification at the 4′ position: 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

Described herein, in some embodiments, is a cell for in vivo synthesis of unnatural polypeptides, the cell comprising: at least two different unnatural codon-anticodon pairs, wherein each unnatural codon-anticodon pair comprises an unnatural codon from unnatural messenger RNA (mRNA) and unnatural anticodon from an unnatural transfer ribonucleic acid (tRNA), said unnatural codon comprising a first unnatural nucleotide and said unnatural anticodon comprising a second unnatural nucleotide; and at least two different unnatural amino acids each covalently linked to a corresponding unnatural tRNA. In some instances, the cell further comprises at least one unnatural DNA molecule comprising at least four unnatural base pairs (UBPs). Described herein, in some embodiments, is a cell for in vivo synthesis of unnatural polypeptides, the cell comprising: at least one unnatural DNA molecule comprising at least four unnatural base pairs, wherein the at least one unnatural DNA molecule encodes (i) a messenger ribonucleic acid (mRNA) molecule encoding an unnatural polypeptide and comprising at least first and second unnatural codons and (ii) at least first and second transfer RNA (tRNA) molecules, the first tRNA molecule comprising a first unnatural anticodon and the second tRNA molecule comprising a second unnatural anticodon, and the at least four unnatural base pairs in the at least one DNA molecule are in sequence contexts such that the first and second unnatural codons of the mRNA molecule are complementary to the first and second unnatural anticodons, respectively. In some embodiments, the cell further comprises the mRNA molecule and the at least first and second tRNA molecules. In some embodiments of the cell, the at least first and second tRNA molecules are covalently linked to unnatural amino acids. In some embodiments, the cell further comprises the unnatural polypeptide.

In some embodiments, the first unnatural nucleotide is positioned at the second or third position of the unnatural codon and is complementarily base paired with the second unnatural nucleotide of the unnatural anticodon. In some instances, the first unnatural nucleotide and the second unnatural nucleotide comprise first and second bases independently selected from the group consisting of

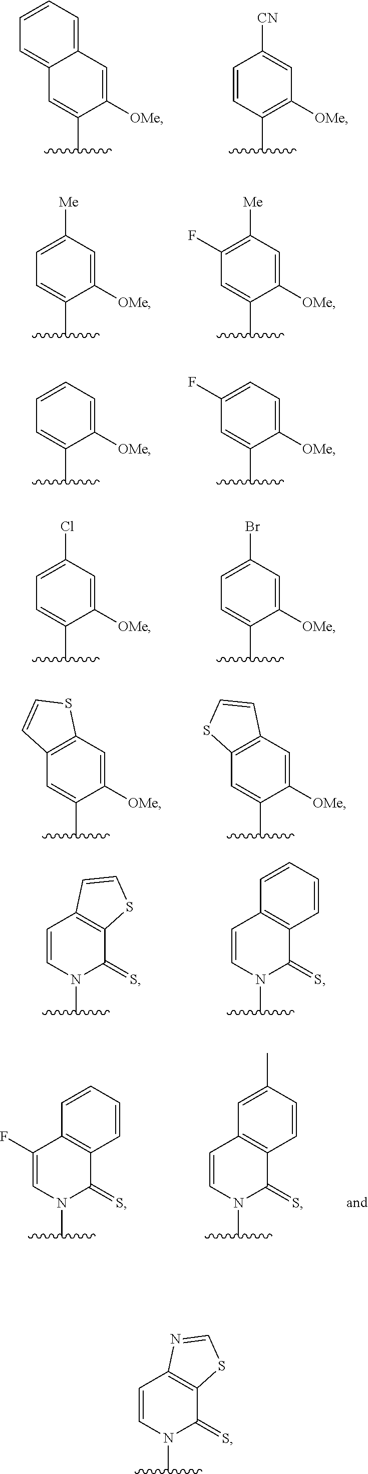

optionally wherein the second base is different from the first base. In some embodiments, the cells further comprise at least one unnatural DNA molecule comprising at least four unnatural base pairs (UBPs). In some cases, the at least four unnatural base pairs are independently selected from the group consisting of dCNMO/dTPT3, dNaM/dTPT3, dCNMO/dTAT1, or dNaM/dTATT. In some instances, the at least one unnatural DNA molecule comprises at least one plasmid. In some embodiments, the at least one unnatural DNA molecule is integrated into genome of the cell. In some embodiments, the at least one unnatural DNA molecule encodes an unnatural polypeptide. In some embodiments, the cells as described herein express a nucleoside triphosphate transporter. In some alternatives, the nucleoside triphosphate transporter comprises the amino acid sequence of PtNTT2. In some cases, the nucleoside triphosphate transporter comprises a truncated amino acid sequence of PtNTT2, optionally wherein the truncated amino acid sequence of PtNTT2 is at least 80% identical to a PtNTT2 encoded by SEQ ID NO.1. In some embodiments, the cells express at least two tRNA synthetases. In some embodiments, the at least two tRNA synthetases are chimeric PylRS (chPylRS) and M. jannaschii AzFRS (MjpAzFRS). In some embodiments, the cells comprise unnatural nucleotides comprising an unnatural sugar moiety. In some instances, the unnatural sugar moiety is selected from the group consisting of: a modification at the 2′ position: OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂F; O-alkyl, S-alkyl, N-alkyl; O-alkenyl, S-alkenyl, N-alkenyl; O-alkynyl, S-alkynyl, N-alkynyl;
O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, -O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —O(CH₂)ₙON[(CH₂)ₙCH₃)]₂, wherein n and m are from 1 to about 10; and/or a modification at the 5′ position: 5′-vinyl, 5′-methyl (R or S); a modification at the 4′ position: 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the cells comprise at least one unnatural nucleotide base that is recognized by an RNA polymerase during transcription. In some embodiments, the cells as described herein translate at least one unnatural polypeptide comprising the at least two unnatural amino acids. In some instances, the at least two unnatural amino acids are independently selected from the group consisting of N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine. In some cases, the cells as described herein are isolated cells. In some alternatives, the cells described herein are prokaryotes. In some cases, the cells described herein comprise a cell line.

### BRIEF DESCRIPTION OF THE DRAWINGS

Various aspects of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 illustrates a workflow using unnatural base pairs (UBPs) to site-specifically incorporate non-canonical amino acids (ncAAs) into an unnatural polypeptide or unnatural protein using an unnatural X-Y base pair. Incorporation of three ncAAs into the unnatural polypeptide or unnatural protein is shown as an example only; any number of ncAAs may be incorporated.

FIG. 2 depicts exemplary unnatural nucleotide base pairs (UBP).

FIG. 3 depicts deoxyribo X analogs. Deoxyribose and phosphates have been omitted for clarity.

FIGS. 4A-B illustrate ribonucleotide analogs. FIG. 4A is a depiction of ribonucleotide X analogs with ribose and phosphates omitted for clarity. FIG. 4B is a depiction of ribonucleotide Y analogs with ribose and phosphates omitted for clarity.

FIGS. 5A-G illustrates exemplary unnatural amino acids. FIG. 5A is adapted from FIG. 2 of Young et al., “Beyond the canonical 20 amino acids: expanding the genetic lexicon,” J. of Biological Chemistry 285(15): 11039-11044 (2010). FIG. 5B is exemplary unnatural amino acid lysine derivatives. FIG. 5C is exemplary unnatural amino acid phenylalanine derivatives. FIG. 5D-5G illustrate exemplary unnatural amino acids. These unnatural amino acids (UAAs) have been genetically encoded in proteins (FIG. 5D—UAA #1-42; FIG. 5E—UAA #43-89; FIG. 5F—UAA #90-128; FIG. 5G—UAA #129-167). FIGS. 5D-5G are adopted from Table 1 of Dumas et al., Chemical Science 2015, 6, 50-69.

FIGS. 6A-D illustrate protein production in non-clonal SSOs using unnatural codons and anticodons. Unnatural codons and unnatural anticodons are written in terms of their DNA coding sequence. FIG. 6A is chemical structure of the dNaM-dTPT3 UBP. FIG. 6B are chemical structures of ncAAs, AzK, PrK, and pAzF. FIG. 6C is schematic illustration of gene cassette used to express sfGFP¹⁵¹(NNN) and M. mazei tRNA^{Pyl}(NNN), where NNN refers to any specified codon or anticodon. FIG. 6D depicts normalized fluorescence from non-clonal SSO cultures at the endpoint of protein expression (i.e. t=180 min after addition of aTc) using specified codons and anticodons both with and without AzK in the media (a.u., arbitrary units). Each replicate culture originates from a different batch of competent SSO starter cells transformed with the UBP carrying plasmid (n=3, biological replicates). Mean with individual data points shown. One representative cropped western blot of purified sfGFP, subjected to SPAAC with TAMRA-PEG₄-DBCO, from SSO cultures shown above each codon and anticodon (only α-GFP channel). FIG. 6D inset is scatterplot of mean endpoint fluorescence in the presence of AzK (from FIG. 6D) versus mean of quantified relative protein shift induced by SPAAC (n=3; biological replicates). Seven top codons chosen for further analyses are encircled.

FIGS. 7A-B illustrate protein production and analyses of codon orthogonality in clonal SSOs. Unnatural codons and unnatural anticodons are written in terms of their DNA coding sequence. FIG. 7A depicts normalized fluorescence from clonal SSOs at the endpoint of protein expression (i.e. t=180 min after addition of aTc) for the seven top codons and anticodons (left) as well as the four other selected codons (right) both with and without AzK. Each replicate culture was propagated from an individual SSO colony (left: n=3, right: n [5, 4, 3, 3]; biological replicates). Mean with individual data points shown. One representative cropped western blot of purified sfGFP, subjected to SPAAC with TAMRA-PEG₄-DBCO from SSO cultures is shown (only α-GFP channel). FIG. 7B depicts normalized fluorescence from clonal SSO cultures at the endpoint of expression for AXC, GXT, and AGX codons and GYT, AYC, and XCT anticodons. All pairwise combinations of both with and without AzK in media, as well as without ribonucleoside triphosphates NaMTP and TPT3TP in the media, were examined. Each culture was propagated from a single colony and mean±standard deviation is indicated (black text; n=3; biological replicates).

FIGS. 8A-F illustrate simultaneous decoding of two unnatural codons. Unnatural codons and unnatural anticodons are written in terms of their DNA coding sequence. FIG. 8A is schematic illustration of gene cassette containing sfGFP^{190,200}(GXT,AXC), M. mazei tRNA^{Pyl}(AYC), and M. jannaschii tRNA^{pAzF}(GYT). FIG. 8B-C, time-course plot of normalized fluorescence during sfGFP expression in the presence of denoted ncAAs. IPTG was added at t=−60 min and aTc was added at t=0. Each replicate expression was carried out in cultures propagated from an individual SSO colony (n 3, biological replicates). Mean and individual data points shown. FIG. 8B illustrates clonal SSO expression of the cassette in FIG. 8A as well as controls showing expression of cassettes containing only single codons with the appropriate tRNA. FIG. 8C illustrates clonal expression of a cassette containing sfGFP^{190,200}(TAA,TAG), M. mazei tRNA^{Pyl}(TTA), and M. jannaschii tRNA^{pAzF}(CTA) also shown, as well as control cassettes containing the single stop-codons with the appropriate suppressor tRNA. FIG. 8D shows pseudocolored western blots of u-GFP and TAMRA fluorescence scans of purified sfGFP from SSOs in FIG. 8B-C, with and without conjugation to TAMRA-PEG₄-DBCO by SPAAC. Images are cropped from the same blots (UBP constructs and stop codon suppressors) but positioned to align the unshifted band in order to ease comparison of electrophoretic migration. FIG. 8E shows the time-course plot of normalized fluorescence during clonal expression of double codon/tRNA cassettes from FIG. 8B-C, with addition of PrK and pAzF. Mean and individual data points shown (n=3, biological replicates). FIG. 8F shows pseudocolored western blots of u-GFP and TAMRA fluorescence scans of purified sfGFP from SSOs in FIG. 8E, with and without conjugation to TAMRA-PEG₄-DBCO by SPAAC and to TAMRA-PEG₄-azide by CuAAC.

FIGS. 9A-C illustrate simultaneous decoding of three unnatural codons. Unnatural codons and unnatural anticodons are written in terms of their DNA coding sequence. FIG. 9A is schematic illustration of gene cassette containing sfGFP^{151; 190,200}(AXC,GXT,AGX), M. mazei tRNA^{Pyl}(XCT), M. jannaschii tRNA^{pAzF}(GYT), and E. coli tRNA^{Ser}(AYC). FIG. 9B is the time-course plot of normalized fluorescence during sfGFP expression in the absence or presence of AzK and/or pAzF. IPTG was added at t=−60 min and aTc was added at t=0. Each replicate expression was carried out in cultures propagated from an individual SSO colony (n=3, biological replicates). Mean and individual data points shown. FIG. 9C is representative deconvoluted mass spectrum from HRMS analysis of intact sfGFP purified from SSOs in FIG. 9B. Peak labels denote molecular weight as well as quantification of each peak relative to other relevant species. Standard single-letter amino acid code used. Mean±standard deviation shown for each of these species (n=3).

FIG. 10 illustrates initial screen of unnatural codons in non-clonal SSOs. Unnatural codons and unnatural anticodons are written in terms of their DNA coding sequence. Paired strip charts of normalized fluorescence from SSO cells at the endpoint of protein expression (i.e. t=180 min after aTc was supplemented) for select codon/anticodon pairs carrying the UBP in either first, second, or third position of the codon. Plus/minus denotes the addition of 20 mM AzK to the media. Each replicate derives from a different batch of competent SSO starter cells (n=3, biological replicates).

FIGS. 11A-B illustrate western blots and fluorescence scans for non-clonal SSO expression. Unnatural codons and unnatural anticodons are written in terms of their DNA coding sequence. FIG. 11A, pseudocolored western blots of u-GFP and TAMRA fluorescence scans of purified sfGFP from cultures in FIG. 6D with conjugation to TAMRA-PEG₄-DBCO by SPAAC. Plus/minus sign denotes if SPAAC was carried out. Three trials carried out (denoted 1, 2, 3; biological replicates). The three trial of each set (NXN/NYN and NNX/XNN) were processed in parallel. FIG. 11B, Quantifications of relative shift in western blots (in FIG. 11A) for specified codon/anticodon pairs (i.e. signal of the shifted band divided by the total signal of both shifted and unshifted bands). plus/minus sign denotes if SPAAC was carried out. Mean standard deviation as well as individual data points shown (n=3).

FIGS. 12A-B illustrate western blots and fluorescence scans for clonal SSO expression. Unnatural codons and unnatural anticodons are written in terms of their DNA coding sequence. FIG. 12A, pseudocolored western blots α-GFP and TAMRA fluorescence scans of purified sfGFP from cultures in FIG. 7A with conjugation to TAMRA-PEG₄-DBCO by SPAAC. Displayed (cropped) area migrated in between 32 kDa and 25 kDa standard protein markers. FIG. 12B, quantifications of relative shift in western blots (in FIG. 12A) for specified codons. Mean±standard deviation as well as individual data points shown (n=3 except n of CXC=5 and n of GXG=4)

FIG. 13 illustrates clonal SSO expressions in the absence of TPT3TP. Unnatural codons and unnatural anticodons are written in terms of their DNA coding sequence. Normalized fluorescence from clonal SSOs at the endpoint of protein expression (i.e. t=180 min after aTc was supplemented) for the top four self-pairing codons/anticodons. Each replicate expression was carried out in cultures propagated from an individual colony as done in FIG. 7A (n=3, biological replicates). Mean±standard deviation shown for both fluorescence and quantified western blot protein shift (i.e. relative shift; gels not shown) as well as individual data points for fluorescence.

FIG. 14 illustrates controls for double codon expressions. Unnatural codons and unnatural anticodons are written in terms of their DNA coding sequence. Time-course plot of normalized fluorescence during sfGFP expressions of specified genotypes, with or without denoted ncAAs in the media. IPTG was added at t=−60 min and aTc was added at t=0. Each replicate expression was carried out in cultures propagated from an individual colony (n=3, biological replicates). Mean and individual data points shown.

FIGS. 15A-B illustrate HRMS analysis of protein from double codon expression. HRMS analysis of intact sfGFP purified from SSOs expressing sfGFP^{151,190,200}(GXT,AXC), tRNA^{Pyl}(AYC), and tRNA^{pAzF}(GYT) with AzK and pAzF in the media, as shown in FIG. 8B (n=3, biological replicates). Standard single-letter amino acid code used. FIG. 15A depicts deconvoluted spectra with annotation of relevant peaks and their relative abundance to each other. FIG. 15B depicts peak assignment and interpretation.

FIGS. 16A-B illustrate HRMS analysis of protein from triple codon expression. HRMS analysis of intact sfGFP purified from SSOs expressing sfGFP^{151,190,200}(AXC,GXT,AGX), tRNA^{Pyl}(XCT), tRNA^{pAzF}(GYT), and tRNA^{Ser}(AYC) with AzK and pAzF in the media, as shown in FIG. 9B (n=3, biological replicates). Standard single-letter amino acid code used.

FIG. 16A depicts deconvoluted spectra with annotation of relevant peaks and their relative abundance to each other. FIG. 16B depicts peak assignment and interpretation.

### DETAILED DESCRIPTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example, a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

Accordingly, a polynucleotide, as the terms are used herein, refer to DNA, RNA, DNA- or RNA-like polymers such as peptide nucleic acids (PNA), locked nucleic acids (LNA), phosphorothioates, unnatural bases, and the like, which are well-known in the art. Polynucleotides can be synthesized in automated synthesizers, e.g., using phosphoroamidite chemistry or other chemical approaches adapted for synthesizer use.

DNA includes, but is not limited to, cDNA and genomic DNA. DNA may be attached, by covalent or non-covalent means, to another biomolecule, including, but not limited to, RNA and peptide. RNA includes coding RNA, e.g. messenger RNA (mRNA). In some embodiments, RNA is rRNA, RNAi, snoRNA, microRNA, siRNA, snRNA, exRNA, piRNA, long ncRNA, or any combination or hybrid thereof. In some instances, RNA is a component of a ribozyme. DNA and RNA can be in any form, including, but not limited to, linear, circular, supercoiled, single-stranded, and double-stranded.

A peptide nucleic acid (PNA) is a synthetic DNA/RNA analog wherein a peptide-like backbone replaces the sugar-phosphate backbone of DNA or RNA. PNA oligomers show higher binding strength and greater specificity in binding to complementary DNAs, with a PNA/DNA base mismatch being more destabilizing than a similar mismatch in a DNA/DNA duplex. This binding strength and specificity also applies to PNA/RNA duplexes. PNAs are not easily recognized by either nucleases or proteases, making them resistant to enzyme degradation. PNAs are also stable over a wide pH range. See also Nielsen P E, Egholm M, Berg R H, Buchardt O (December 1991). “Sequence-selective recognition of DNA by strand displacement with a thymine-substituted polyamide”, Science 254 (5037): 1497-500. doi:10.1126/science.1962210. PMID 1962210; and, Egholm M, Buchardt O, Christensen L, Behrens C, Freier S M, Driver D A, Berg R H, Kim S K, Norden B, and Nielsen P E (1993), “PNA Hybridizes to Complementary Oligonucleotides Obeying the Watson-Crick Hydrogen Bonding Rules”. Nature 365 (6446): 566-8. doi:10.1038/365566a0. PMID 7692304

A locked nucleic acid (LNA) is a modified RNA nucleotide, wherein the ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers can be synthesized chemically and are commercially available. The locked ribose conformation enhances base stacking and backbone pre-organization. See, for example, Kaur, H; Arora, A; Wengel, J; Maiti, S (2006), “Thermodynamic, Counterion, and Hydration Effects for the Incorporation of Locked Nucleic Acid Nucleotides into DNA Duplexes”, Biochemistry 45 (23): 7347-55. doi:10.1021/bi060307w. PMID 16752924; Owczarzy R.; You Y., Groth C. L., Tataurov A. V. (2011), “Stability and mismatch discrimination of locked nucleic acid-DNA duplexes.”, Biochem. 50 (43): 9352-9367. doi:10.1021/bi200904e. PMC 3201676. PMID 21928795; Alexei A. Koshkin; Sanjay K. Singh, Poul Nielsen, Vivek K. Rajwanshi, Ravindra Kumar, Michael Meldgaard, Carl Erik Olsen, Jesper Wengel (1998), “LNA (Locked Nucleic Acids): Synthesis of the adenine, cytosine, guanine, 5-methylcytosine, thymine and uracil bicyclonucleoside monomers, oligomerisation, and unprecedented nucleic acid recognition”, Tetrahedron 54 (14): 3607-30. doi:10.1016/50040-4020(98)00094-5; and, Satoshi Obika; Daishu Nanbu, Yoshiyuki Hari, Ken-ichiro Mono, Yasuko In, Toshimasa Ishida, Takeshi Imanishi (1997), “Synthesis of 2′-O,4′-C-methyleneuridine and -cytidine. Novel bicyclic nucleosides having a fixed C3′-endo sugar puckering”, Tetrahedron Lett. 38 (50): 8735-8. doi:10.1016/S0040-4039(97)10322-7.

A molecular beacon or molecular beacon probe is an oligonucleotide hybridization probe that can detect the presence of a specific nucleic acid sequence in a homogenous solution. Molecular beacons are hairpin shaped molecules with an internally quenched fluorophore whose fluorescence is restored when they bind to a target nucleic acid sequence. See, for example, Tyagi S, Kramer F R (1996), “Molecular beacons: probes that fluoresce upon hybridization”, Nat Biotechnol. 14 (3): 303-8. PMID 9630890; Tapp I, Malmberg L, Rennel E, Wik M, Syvanen A C (2000 April), “Homogeneous scoring of single-nucleotide polymorphisms: comparison of the 5′-nuclease TaqMan assay and Molecular Beacon probes”, Biotechniques 28 (4): 732-8. PMID 10769752; and, Akimitsu Okamoto (2011), “ECHO probes: a concept of fluorescence control for practical nucleic acid sensing”, Chem. Soc. Rev. 40: 5815-5828.

In some embodiments, a nucleobase is generally the heterocyclic base portion of a nucleoside. Nucleobases may be naturally occurring, may be modified, may bear no similarity to natural bases, and may be synthesized, e.g., by organic synthesis. In certain embodiments, a nucleobase comprises any atom or group of atoms capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, are referred to as nucleobases for simplicity. In some embodiments, when referring to a nucleobase, a “(d)” indicates that the nucleobase can be attached to a deoxyribose or a ribose.

In some embodiments, a nucleoside is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

In some embodiments, the unnatural mRNA codons and unnatural tRNA anticodons as described in the present disclosure can be written in terms of their DNA coding sequence. For example, unnatural tRNA anticodon can be written as GYU or GYT.

The section headings used herein are for organizational purposes only and are not to be construed as limiting the subject matter described.

### Compositions and Methods for In Vivo Synthesis of Unnatural Polypeptides

Disclosed herein are compositions and methods for in vivo synthesis of unnatural polypeptides with an expanded genetic alphabet. In some instances, the compositions and methods as described herein comprise an unnatural nucleic acid molecule encoding an unnatural polypeptide, wherein the unnatural polypeptide comprises an unnatural amino acid. In some instances, the unnatural polypeptide comprises at least two unnatural amino acids. In some cases, the unnatural polypeptide comprises at least three unnatural amino acids. In some instances, the unnatural polypeptide comprises two unnatural amino acids. In some cases, the unnatural polypeptide comprises three unnatural amino acids. In some instances, the at least two unnatural amino acids being incorporated into the unnatural polypeptide can be the same or different unnatural amino acids. In some cases, the unnatural amino acids are incorporated into the unnatural polypeptide in a site-specific manner. In some cases, the unnatural polypeptide is an unnatural protein.

In some cases, the compositions and methods as described herein comprise a semi-synthetic organism (SSO). In some instances, the methods comprise incorporating at least one unnatural base pair (UBP) into at least one unnatural nucleic acid molecule. In some embodiments, the methods comprise incorporating one UBP into the at least one unnatural nucleic acid molecule. In some embodiments, the methods comprise incorporating two UBPs into the at least one unnatural nucleic acid molecule. In some embodiments, the methods comprise incorporating three UBPs into the at least one unnatural nucleic acid molecule. UBP base pairs are formed by pairing between the unnatural nucleobases of two unnatural nucleosides. In some embodiments, the unnatural nucleic acid molecule is an unnatural DNA molecule.

In some embodiments, the at least one unnatural nucleic acid molecule is or comprises one molecule (e.g., a plasmid or a chromosome). In some embodiments, the at least one unnatural nucleic acid molecule is or comprises two molecules (e.g., two plasmids, two chromosomes, or a chromosome and a plasmid). In some embodiments, the at least one unnatural nucleic acid molecule is or comprises three molecules (e.g., three plasmids, two plasmids and a chromosome, a plasmid and two chromosomes, or three chromosomes). Examples of chromosomes include genomic chromosomes into which a UBP has been integrated and artificial chromosomes (e.g., bacterial artificial chromosomes) comprising a UBP. In some embodiments, where at least one unnatural DNA molecule comprising at least four unnatural base pairs is used and the at least one unnatural DNA molecule is two or more molecules, the at least four unnatural base pairs may be distributed among the two or more molecules in any feasible manner (e.g., one in the first and three in the second, two in the first and two in the second, etc.).

In some instances, the at least one unnatural nucleic acid molecule, optionally including the UBPs, is transcribed to afford a messenger RNA molecule comprising at least one unnatural codon harboring at least one unnatural nucleotide. In some embodiments, transcribing refers to generating one or more RNA molecules complementary to a portion of a DNA molecule. In some cases, the unnatural nucleotide occupies the first, second, or third codon position of the unnatural codon, e.g., the second or third codon position. In some cases, two unnatural nucleotides occupy first and second, first and third, second and third, or first and third codon positions of the unnatural codon. In some cases, three unnatural nucleotides occupy all three codon positions of the unnatural codon. In some cases, the mRNA harboring the unnatural nucleotides comprises at least two unnatural codons (in some embodiments, the expression “at least two unnatural codons” is interchangeable with “at least first and second unnatural codons”). In some cases, the mRNA harboring the unnatural nucleotides comprises two unnatural codons. In some cases, the mRNA harboring the unnatural nucleotides comprises three unnatural codons.

In some embodiments, the unnatural nucleic acid molecule, optionally including the UBPs, is transcribed to afford at least one tRNA molecule, where the tRNA molecule comprises an unnatural anticodon harboring at least one unnatural nucleotide. In some cases, an unnatural nucleotide occupies the first, second, or third anticodon position of the unnatural anticodon. In some cases, two unnatural nucleotides occupy first and second, first and third, second and third, or first and third anticodon positions of the unnatural anticodon. In some cases, three unnatural nucleotides occupy all three anticodon positions of the unnatural anticodon. In some cases, the unnatural nucleic acid molecule, optionally including the UBPs, is transcribed to afford at least two tRNAs comprising at least two unnatural anticodons. In cases, the at least two unnatural anticodons can be the same or different. In some instances, the unnatural nucleic acid molecule, optionally including the UBPs, is transcribed to afford two tRNAs comprising unnatural anticodons that can be the same or different. In some instances, the unnatural nucleic acid molecule, optionally including the UBPs, is transcribed to afford three tRNAs comprising three unnatural anticodons that can be the same or different.

In some embodiments, the at least one unnatural codon encoded by the mRNA can be complementary to the at least unnatural anticodon of the tRNA to form an unnatural codon-anticodon pair. In some cases, the compositions and methods described herein comprise synthesizing the unnatural polypeptide with one, two, three, or more unnatural codon-anticodon pairs. In some cases, the compositions and methods described herein comprise synthesizing the unnatural polypeptide with two unnatural codon-anticodon pairs. In some cases, the compositions and methods described herein comprise synthesizing the unnatural polypeptide with three unnatural codon-anticodon pairs.

In some cases, the compositions and methods described herein comprise synthesizing the unnatural polypeptide with one, two, three, or more unnatural amino acids using one, two, three, or more unnatural codon-anticodon pairs. In some cases, the compositions and methods described herein comprise synthesizing the unnatural polypeptide with two unnatural amino acids using two unnatural codon-anticodon pairs. In some cases, the compositions and methods described herein comprise synthesizing the unnatural polypeptide with three unnatural amino acids using three unnatural codon-anticodon pairs.

In some instances, the unnatural codon comprises a nucleic acid sequence XNN, NXN, NNX, XXN, XNX, NXX, or XXX, and the unnatural anticodon comprises a nucleic acid sequence XNN, YNN, NXN, NYN, NNX, NNY, NXX, NYY, XNX, YNY, XXN, YYN, or YYY to form the unnatural codon-anticodon pair. In some cases, the unnatural codon-anticodon pair comprises of NNX-XNN, NNX-YNN, or NXN-NYN, where N is any natural nucleotide, X is a first unnatural nucleotide, and Y is a second unnatural nucleotide. In some embodiments, any natural nucleotide includes nucleotides having a standard base such as adenine, thymine, uracil, guanine, or cytosine, and nucleotides having a naturally occurring modified base such as pseudouridine, 5-methylcytosine, etc. In some embodiments, the unnatural codon-anticodon pair comprises at least one G in the codon and at least one C in the anticodon. In some embodiments, the unnatural codon-anticodon pair comprises at least one G or C in the codon and at least one complementary C or G in the anticodon. X and Y are each independently selected from a group consisting of (i) 2-thiouracil, 2′-deoxyuridine, 4-thio-uracil, uracil-5-yl, hypoxanthin-9-yl (I), 5-halouracil; 5-propynyl-uracil, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, dihydrouracil, 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1,4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, 6-aza-adenine, 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, 6-aza-guanine, hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

In some embodiments, the X and Y are independently selected from a group consisting of:

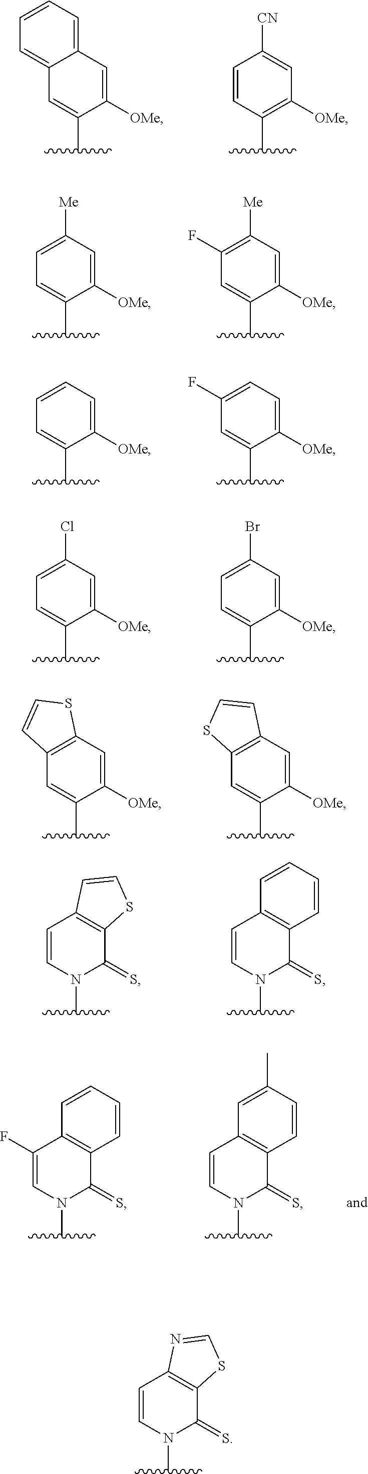

In some cases, the unnatural codon-anticodon pair comprises NNX-XNN, where NNX-XNN is selected from the group consisting of AAX-XUU, AUX-XAU, ACX-XGU, AGX-XCU, UAX-XUA, UUX-XAA, UCX-XGA, UGX-XCA, CAX-XUG, CUX-XAG, CCX-XGG, CGX-XCG, GAX-XUC, GUX-XAC, GCX-XGC, and GGX-XCC. In some cases, the unnatural codon-anticodon pair comprises NNX-YNN, where NNX-YNN is selected from the group consisting of AAX-YUU, AUX-YAU, ACX-YGU, AGX-YCU, UAX-YUA, UUX-YAA, UCX-YGA, UGX-YCA, CAX-YUG, CUX-YAG, CCX-YGG, CGX-YCG, GAX-YUC, GUX-YAC, GCX-YGC, and GGX-YCC. In some embodiments, the unnatural codon-anticodon pair comprises NXN-NXN, where NXN-NXN is selected from the group consisting of AXA-UXU, AXU-AXU. AXC-GXU, AXG-CXU, UXA-UXA, UXU-AXA, UXC-GXA, UXG-CXA, CXA-UXG, CXU-AXG, CXC-GXG, CXG-CXG, GXA-UXC, GXU-AXC, GXC-GXC, and GXG-CXC. In some instances, the unnatural codon-anticodon pair comprises NXN-NYN, where NXN-NYN is selected from the group consisting of AXA-UYU, AXU-AYU. AXC-GYU, AXG-CYU, UXA-UYA, UXU-AYA, UXC-GYA, UXG-CYA, CXA-UYG, CXU-AYG, CXC-GYG, CXG-CYG, GXA-UYC, GXU-AYC, GXC-GYC, and GXG-CYC.

In some embodiments, the unnatural codon-anticodon pair comprises XNN-NNX, where XNN-NNX is selected from the group consisting of XAA-UUX, XAU-AUX, XAC-AGX, XAG-CUX, XUA-UAX, XUU-AAX, XUC-GAX, XUG-CAX, XCA-UGX, XCU-AGX, XCC-GGX, XCG-CGX, XGA-UCX, XGU-ACX, XGC-GCX, and XGG-CCX. In some embodiments, the unnatural codon-anticodon pair comprises XNN-NNY, where XNN-NNY is selected from the group consisting of XAA-UUY, XAU-AUY, XAC-AGY, XAG-CUY, XUA-UAY, XUU-AAY, XUC-GAY, XUG-CAY, XCA-UGY, XCU-AGY, XCC-GGY, XCG-CGY, XGA-UCY, XGU-ACY, XGC-GCY, and XGG-CCY.

In some embodiments, the unnatural codon-anticodon pair comprises XXN-NXX, where XXN-NXX is selected from the group consisting of XXA-UXX, XXU-AXX, XXC-GXX, and XXG-CXX. In some embodiments, the unnatural codon-anticodon pair comprises XXN-NYY, where XXN-NYY is selected from the group consisting of XXA-UYY, XXU-AYY, XXC-GYY, and XXG-CYY. In some alternatives, the unnatural codon-anticodon pair comprises XNX-XNX, where XNX-XNX is selected from the group consisting of XAX-XUX, XUX-XAX, XCX-XGX, and XGX-XCX. In some embodiments, the unnatural codon-anticodon pair comprises XNX-YNY, where XNX-YNY is selected from the group consisting of XAX-YUY, XUX-YAY, XCX-YGY, and XGX-YCY. In some cases, the unnatural codon-anticodon pair comprises NXX-XXN, where NXX-XXN is selected from the group consisting of AXX-XXU, UXX-XXA, CXX-XXG, and GXX-XXC. In some instances, the unnatural codon-anticodon pair comprises NXX-YYN, where NXX-YYN is selected from the group consisting of AXX-YYU, UXX-YYA, CXX-YYG, and GXX-YYC. In some cases, the unnatural codon-anticodon pair comprises XXX-XXX or XXX-YYY.

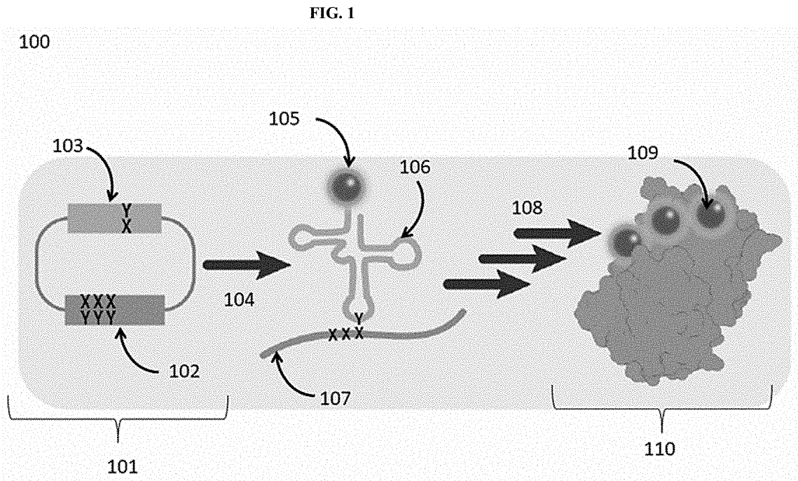

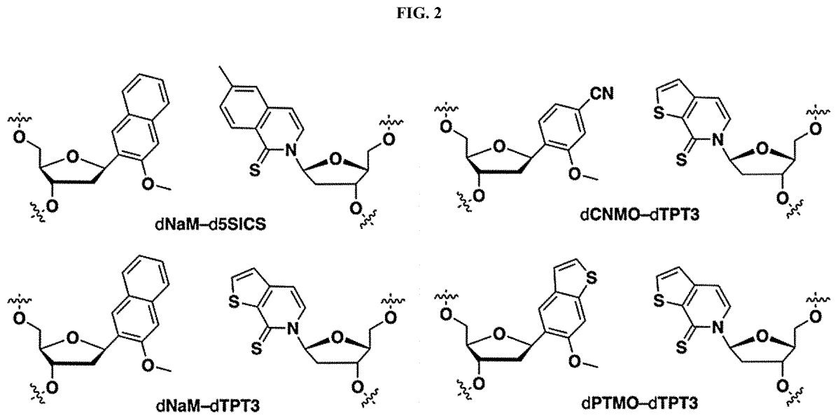

In an exemplary workflow 100 (FIG. 1) of a method producing an unnatural polypeptide with an expanded genetic alphabet (FIG. 2), DNA 101 coding for a protein 102 and a tRNA 103, each comprising complementary unnatural nucleobases (X, Y) is transcribed 104 to generate a tRNA 106 and mRNA 107. X is a first unnatural nucleotide and Y is a second unnatural nucleotide. After charging the tRNA with an unnatural amino acid 105, the mRNA 107 is translated 108 to generate a protein 110 comprising one or more unnatural amino acids 109. Methods and compositions described herein in some instances allow for site-specific incorporation of unnatural amino acids with high fidelity and yield. Also described herein are semi-synthetic organisms comprising an expanded genetic alphabet, methods for using the semi-synthetic organisms to produce protein products, including those comprising at least one unnatural amino acid residue.

Selection of unnatural nucleobases allows for optimization of one or more steps in the methods described herein. For example, nucleobases are selected for high efficiency replication, transcription, and/or translation. In some instances, more than one unnatural nucleobase pair is utilized for the methods described herein. For example, a first set of nucleobases comprising a deoxyribo moiety are used for DNA replication (such as a first nucleobase and a second nucleobase, configure to form a first base pair), and a second set of nucleobases (such a third nucleobase and a fourth nucleobase, wherein the third and fourth nucleobases are attached to ribose, configured to form a second base pair) are used for transcription/translation. Complementary pairing between a nucleobase of the first set and a nucleobase of the second set in some instances allow for transcription of genes to generate tRNA or proteins from a DNA template comprising nucleobases from the first set. Complementary pairing between nucleobases of the second set (second base pair) in some instances allows for translation by matching tRNAs comprising unnatural nucleic acids and mRNA. In some cases, nucleobases in the first set are attached to a deoxyribose moiety. In some cases, nucleobases in the first set are attached to ribose moiety. In some instances, nucleobases of both sets are unique. In some instances, at least one nucleobase is the same in both sets. In some instances, a first nucleobase and a third nucleobase are the same. In some embodiments, the first base pair and the second base pair are not the same. In some cases, the first base pair, the second base pair, and the third base pair are not the same.

In some embodiments, yield of unnatural polypeptide or unnatural protein synthesized by the compositions and methods as disclosed herein is higher compared to yield of the same unnatural polypeptide or unnatural protein synthesized by other methods. In some instances, the yield of unnatural polypeptide or unnatural protein synthesized by the compositions and methods as disclosed herein is at least 10%, at least 20%, at least 30%, at least 40%, or at least 50% higher than the yield of the same unnatural polypeptide or unnatural protein synthesized by other methods. An example of other methods includes methods utilizing amber codon suppression.

In some instance, solubility of unnatural polypeptide or unnatural protein synthesized by the compositions and methods as disclosed herein is higher compared the solubility of the same unnatural polypeptide or unnatural protein synthesized by other methods. In some instances, the solubility of unnatural polypeptide or unnatural protein synthesized by the compositions and methods as disclosed herein is at least 10%, at least 20%, at least 30%, at least 40%, or at least 50% higher than the same unnatural polypeptide or unnatural protein synthesized by other methods. In some cases, biological activity of unnatural protein synthesized by the compositions and methods as disclosed herein is higher compared to biological activity of the same unnatural protein synthesized by other methods. In some instances, the biological activity of the unnatural protein synthesized by the compositions and methods as disclosed herein is at least 10%, at least 20%, at least 30%, at least 40%, or at least 50% higher than the biological activity of the same unnatural protein synthesized by other methods.

In some embodiments, the compositions and methods for in vivo synthesis of unnatural polypeptides as described herein utilize or comprise a semi-synthetic organism (SSO). In some embodiments, the SSO is undergoing clonal expansion during the synthesis of the unnatural polypeptides. In some instances, the SSO is not clonal expanding during the synthesis of the unnatural polypeptides. In some cases, the SSO can be arrested at any phase of the cell cycle during the synthesis of the unnatural polypeptides. In some embodiments, the compositions and methods as described herein can synthesize the unnatural polypeptides in vitro. In some cases, the compositions and methods as described herein can comprise a cell-free system to synthesize the unnatural polypeptides.

### Nucleic Acid Molecules

In some embodiments, a nucleic acid (e.g., also referred to herein as nucleic acid molecule of interest) is from any source or composition, such as DNA, cDNA, gDNA (genomic DNA), RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For natural RNA, the uracil base is uridine. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism. In some embodiments, an unnatural nucleotide is not a natural nucleotide. In some embodiments, a nucleotide that does not comprise a natural base comprises an unnatural nucleobase.

### Unnatural Nucleic Acids

A nucleotide analog, or unnatural nucleotide, comprises a nucleotide which contains some type of modification to either the base, sugar, or phosphate moieties. In some embodiments, a modification comprises a chemical modification. In some cases, modifications occur at the 3′OH or 5′OH group, at the backbone, at the sugar component, or at the nucleotide base. Modifications, in some instances, optionally include non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules. In one aspect, a modified backbone comprises a backbone other than a phosphodiester backbone. In one aspect, a modified sugar comprises a sugar other than deoxyribose (in modified DNA) or other than ribose (modified RNA). In one aspect, a modified base comprises a base other than adenine, guanine, cytosine or thymine (in modified DNA) or a base other than adenine, guanine, cytosine or uracil (in modified RNA).

In some embodiments, the nucleic acid comprises at least one modified base. In some instances, the nucleic acid comprises 2, 3, 4, 5, 6, 7, 8, 9, 10, 15, 20, or more modified bases. In some cases, modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

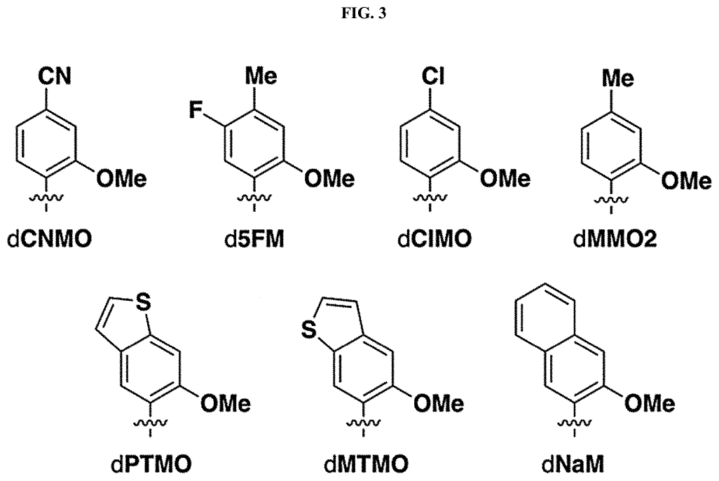

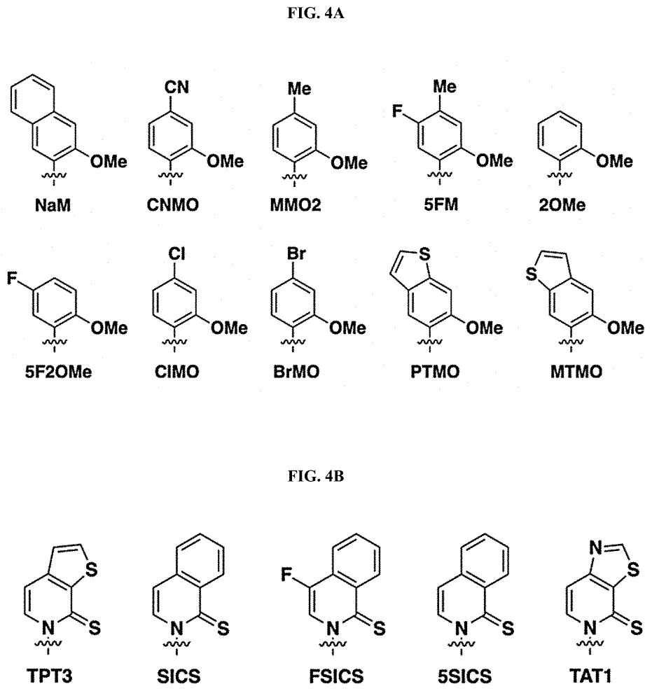

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613. In some instances, an unnatural nucleic acid comprises a nucleobase of FIG. 3. In some instances, an unnatural nucleic acid comprises a nucleobase of FIG. 4A. In some instances, an unnatural nucleic acid comprises a nucleobase of FIG. 4B.

Unnatural nucleic acids comprising various heterocyclic bases and various sugar moieties (and sugar analogs) are available in the art, and the nucleic acid in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d]pyrimidin-5-yl, 2-amino-4-oxopyrolo [2, 3-d] pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, a modified base of an unnatural nucleic acid is depicted below, wherein the wavy line or R identifies a point of attachment to the deoxyribose or ribose.

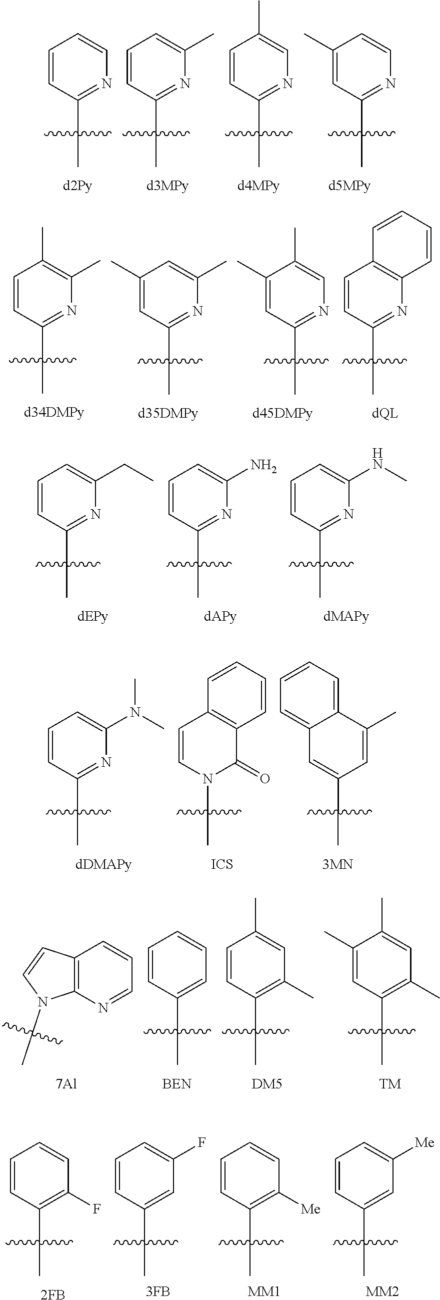

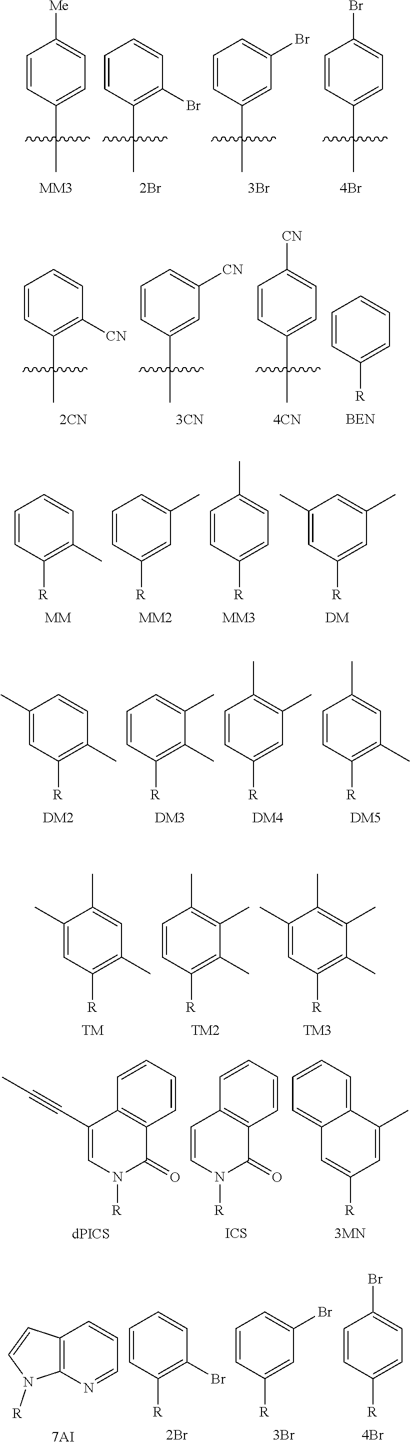

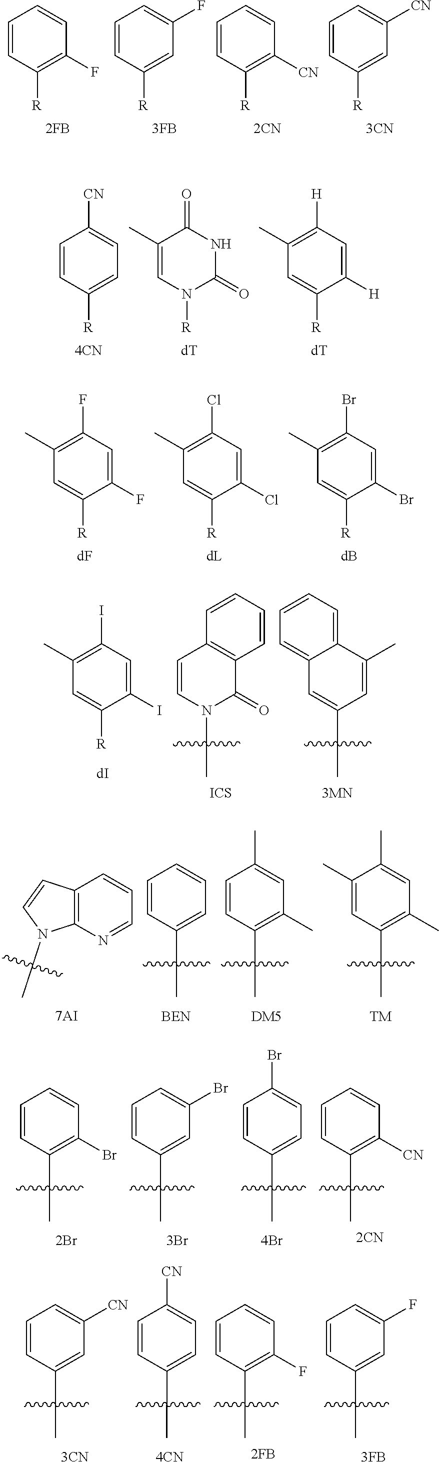

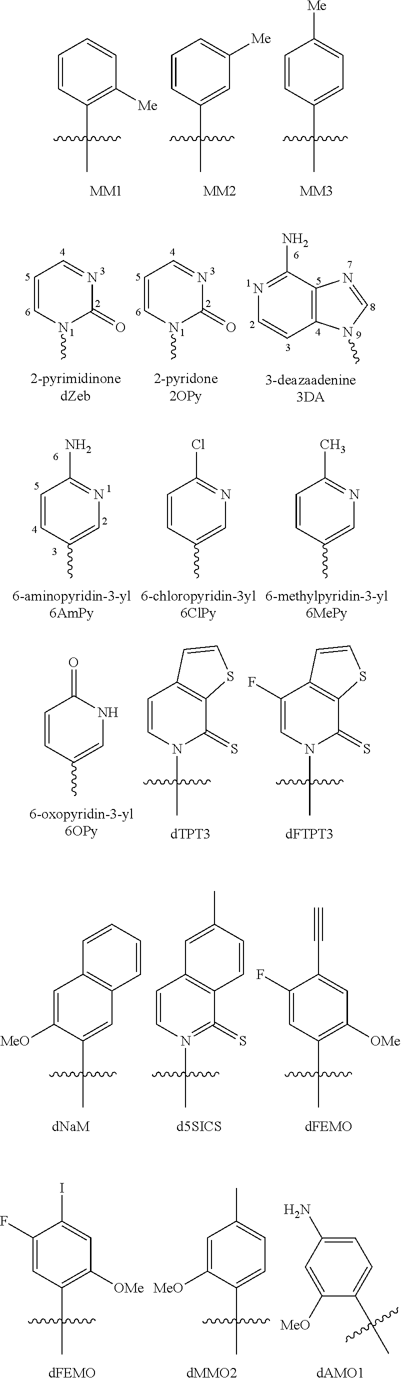

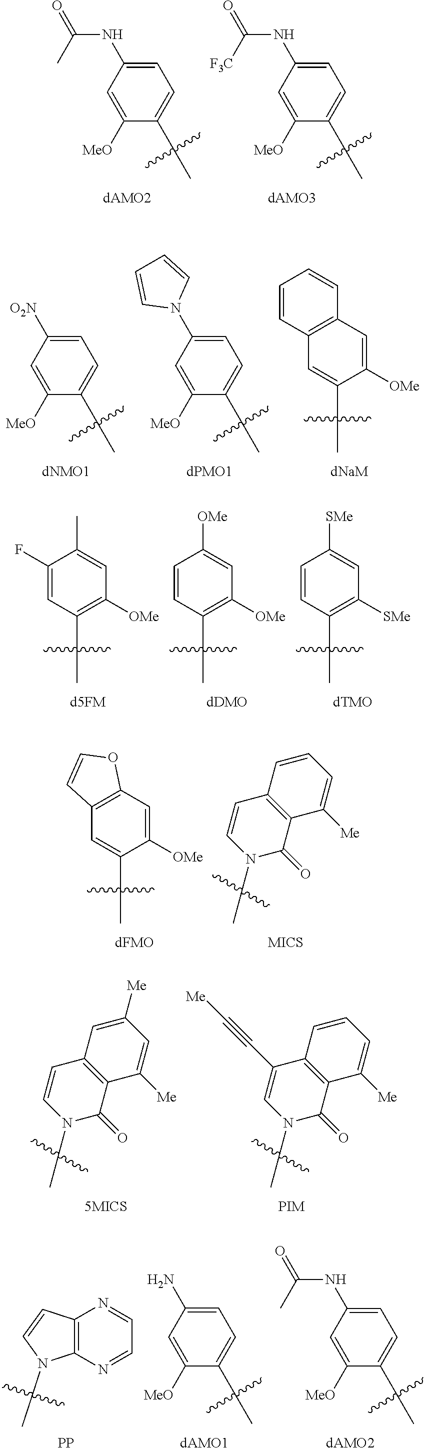

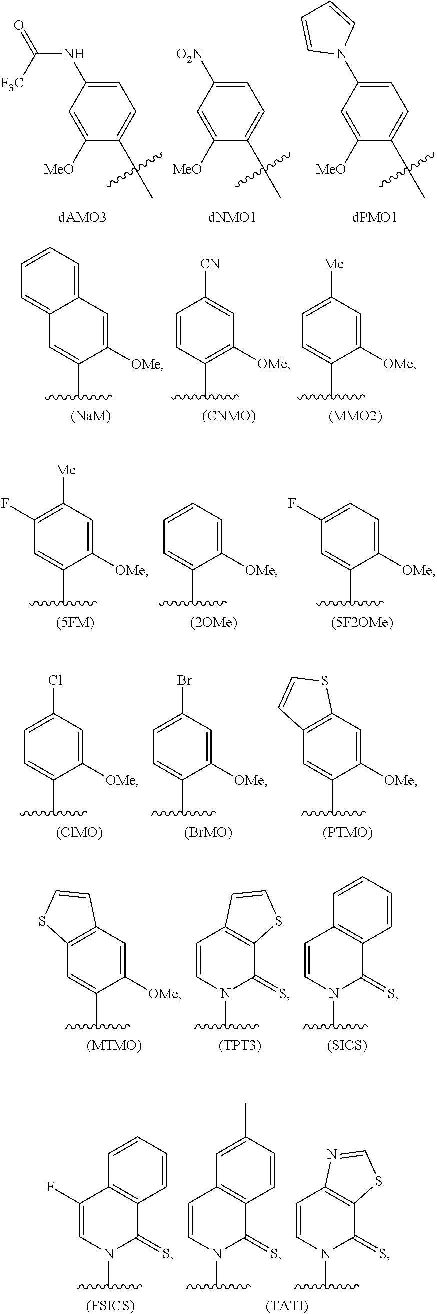

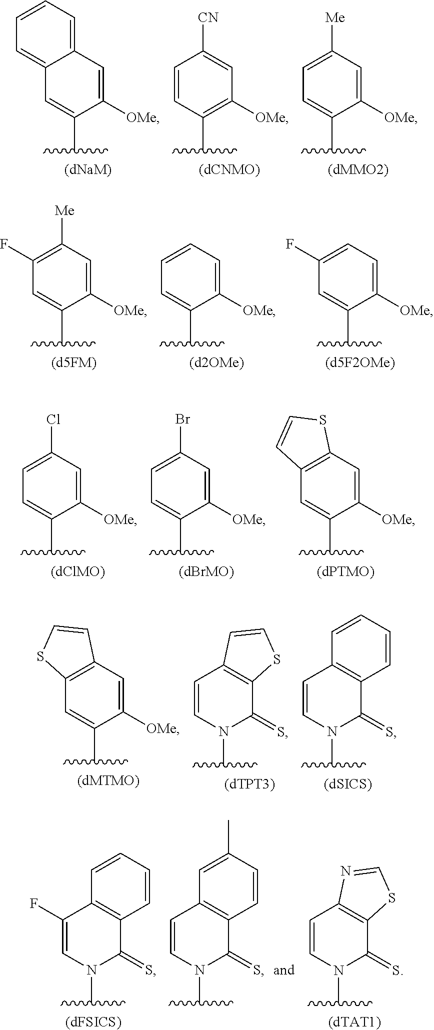

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)-CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R1)(R2) (R═H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]2, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O- 2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, N.Y.; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Described herein are nucleobases used in the compositions and methods for replication, transcription, translation, and incorporation of unnatural amino acids into proteins. In some embodiments, a nucleobase described herein comprises the structure:

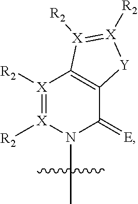

wherein each X is independently carbon or nitrogen; R₂ is optional and when present is independently hydrogen, alkyl, alkenyl, alkynyl; methoxy, methanethiol, methaneseleno, halogen, cyano, or azide group; wherein each Y is independently sulfur, oxygen, selenium, or secondary amine; wherein each E is independently oxygen, sulfur or selenium; and wherein the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, connected to a mono-phosphate, diphosphate, or triphosphate group, optionally comprising an u-thiotriphosphate, β-thiotriphosphate, or γ-thiotriphosphate group, or is included in an RNA or a DNA or in an RNA analog or a DNA analog. In some embodiments, R₂ is lower alkyl (e.g., C₁-C₆), hydrogen, or halogen. In some embodiments of a nucleobase described herein, R₂ is fluoro. In some embodiments of a nucleobase described herein, X is carbon. In some embodiments of a nucleobase described herein, E is sulfur. In some embodiments of a nucleobase described herein, Y is sulfur. In some embodiments of a nucleobase described herein, a nucleobase has the structure:

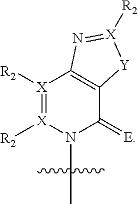

In some embodiments of a nucleobase described herein, E is sulfur and Y is sulfur. In some embodiments of a nucleobase described herein, the wavy line indicates a point of bonding to a ribosyl or deoxyribosyl moiety. In some embodiments of a nucleobase described herein, the wavy line indicates a point of bonding to a ribosyl or deoxyribosyl moiety, connected to a triphosphate group. In some embodiments of a nucleobase described herein is a component of a nucleic acid polymer. In some embodiments of a nucleobase described herein, the nucleobase is a component of a tRNA. In some embodiments of a nucleobase described herein, the nucleobase is a component of an anticodon in a tRNA. In some embodiments of a nucleobase described herein, the nucleobase is a component of an mRNA. In some embodiments of a nucleobase described herein, the nucleobase is a component of a codon of an mRNA. In some embodiments of a nucleobase described herein, the nucleobase is a component of RNA or DNA. In some embodiments of a nucleobase described herein, the nucleobase is a component of a codon in DNA. In some embodiments of a nucleobase described herein, the nucleobase forms a nucleobase pair with another complementary nucleobase.

### Nucleic Acid Base Pairing Properties

In some embodiments, an unnatural nucleotide forms a base pair (an unnatural base pair; UBP) with another unnatural nucleotide during or after incorporation into DNA or RNA. In some embodiments, a stably integrated unnatural nucleic acid is an unnatural nucleic acid that can form a base pair with another nucleic acid, e.g., a natural or unnatural nucleic acid. In some embodiments, a stably integrated unnatural nucleic acid is an unnatural nucleic acid that can form a base pair with another unnatural nucleic acid (unnatural nucleic acid base pair (UBP)). For example, a first unnatural nucleic acid can form a base pair with a second unnatural nucleic acid. For example, one pair of unnatural nucleoside triphosphates that can base pair during and after incorporation into nucleic acids include a triphosphate of (d)5SICS ((d)5SICSTP) and a triphosphate of (d)NaM ((d)NaMTP). Other examples include but are not limited to: a triphosphate of (d)CNMO ((d)CNMOTP) and a triphosphate of (d)TPT3 ((d)TPT3TP). Such unnatural nucleotides can have a ribose or deoxyribose sugar moiety (indicated by the “(d)”). For example, one pair of unnatural nucleoside triphosphates that can base pair when incorporated into nucleic acids includes a triphosphate of TAT1 (TAT1TP) and a triphosphate of NaM (NaMTP). In some embodiments, one pair of unnatural nucleoside triphosphates that can base pair when incorporated into nucleic acids includes a triphosphate of dCNMO (dCNMOTP) and a triphosphate of TAT1 (TAT1TP). In some embodiments, one pair of unnatural nucleoside triphosphates that can base pair when incorporated into nucleic acids includes a triphosphate of dTPT3 (dTPT3TP) and a triphosphate of NaM (NaMTP). In some embodiments, an unnatural nucleic acid does not substantially form a base pair with a natural nucleic acid (A, T, G, C). In some embodiments, a stably integrated unnatural nucleic acid can form a base pair with a natural nucleic acid.

In some embodiments, a stably integrated unnatural (deoxy)ribonucleotide is an unnatural (deoxy)ribonucleotide that can form a UBP but does not substantially form a base pair with each any of the natural (deoxy)ribonucleotides. In some embodiments, a stably integrated unnatural (deoxy)ribonucleotide is an unnatural (deoxy)ribonucleotide that can form a UBP but does not substantially form a base pair with one or more natural nucleic acids. For example, a stably integrated unnatural nucleic acid may not substantially form a base pair with A, T, and, C, but can form a base pair with G. For example, a stably integrated unnatural nucleic acid may not substantially form a base pair with A, T, and, G, but can form a base pair with C. For example, a stably integrated unnatural nucleic acid may not substantially form a base pair with C, G, and, A, but can form a base pair with T. For example, a stably integrated unnatural nucleic acid may not substantially form a base pair with C, G, and, T, but can form a base pair with A. For example, a stably integrated unnatural nucleic acid may not substantially form a base pair with A and T, but can form a base pair with C and G. For example, a stably integrated unnatural nucleic acid may not substantially form a base pair with A and C, but can form a base pair with T and G. For example, a stably integrated unnatural nucleic acid may not substantially form a base pair with A and G, but can form a base pair with C and T. For example, a stably integrated unnatural nucleic acid may not substantially form a base pair with C and T, but can form a base pair with A and G. For example, a stably integrated unnatural nucleic acid may not substantially form a base pair with C and G, but can form a base pair with T and G. For example, a stably integrated unnatural nucleic acid may not substantially form a base pair with T and G, but can form a base pair with A and G. For example, a stably integrated unnatural nucleic acid may not substantially form a base pair with, G, but can form a base pair with A, T, and, C. For example, a stably integrated unnatural nucleic acid may not substantially form a base pair with, A, but can form a base pair with G, T, and, C. For example, a stably integrated unnatural nucleic acid may not substantially form a base pair with, T, but can form a base pair with G, A, and, C. For example, a stably integrated unnatural nucleic acid may not substantially form a base pair with, C, but can form a base pair with G, T, and, A.

Exemplary unnatural nucleotides capable of forming an unnatural DNA or RNA base pair (UBP) under conditions in vivo includes, but is not limited to, 5SICS, d5SICS, NaM, dNaM, dTPT3, dMTMO, dCNMO, TAT1, and combinations thereof. In some embodiments, unnatural nucleotide base pairs include but are not limited to:

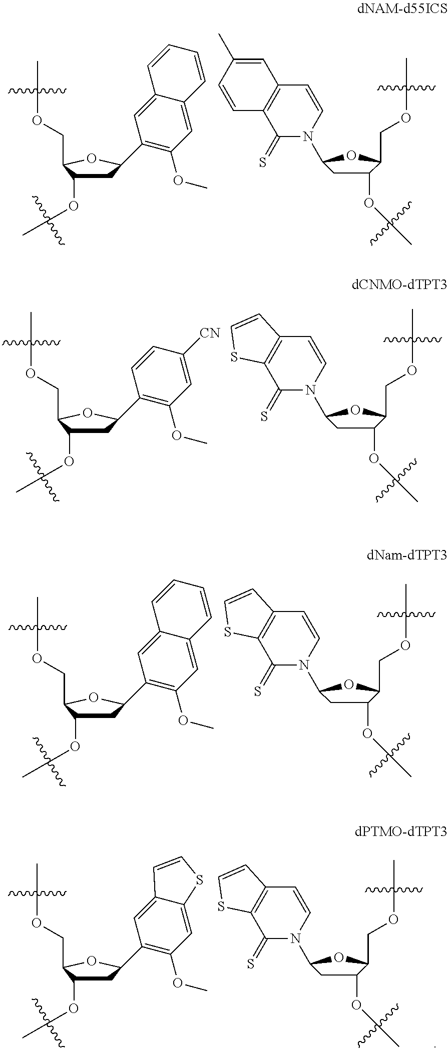

### Engineered Organisms

In some embodiments, methods and plasmids disclosed herein are further used to generate engineered organism, e.g. an organism that incorporates and replicates an unnatural nucleotide or an unnatural nucleic acid base pair (UBP) and may also use the nucleic acid containing the unnatural nucleotide to transcribe mRNA and tRNA which are used to translate unnatural polypeptides or unnatural proteins containing at least one unnatural amino acid residue. In some cases, the unnatural amino acid residue is incorporated into the unnatural polypeptide or unnatural protein in a site-specific manner. In some instances, the organism is a non-human semi-synthetic organism (SSO). In some instances, the organism is a semi-synthetic organism (SSO). In some instances, the SSO is a cell. In some instances, the in vivo methods comprise a semi-synthetic organism (SSO). In some instances, the semi-synthetic organism comprises a microorganism. In some instances, the organism comprises a bacterium. In some instances, the organism comprises a gram-negative bacterium. In some instances, the organism comprises a gram-positive bacterium. In some instances, the organism comprises an Escherichia coli. Such modified organisms variously comprise additional components, such as DNA repair machinery, modified polymerases, nucleotide transporters, or other components. In some instances, the SSO comprises E. coli strain YZ3. In some instances, the SSO comprises E. coli strain ML1 or ML2, such as those strains described in FIG. 1 (B-D) of Ledbetter, et al. J. Am Chem. Soc. 2018, 140(2), 758. In some cases, the SSO is a cell line. In some cases, the cell line is immortalized cell line. In some instances, the cell line comprises primary cells. In some instances, the cell line comprises stem cells. In some intendances, the SSO is an organoid.

In some instances, the cell employed is genetically transformed with an expression cassette encoding a heterologous protein, e.g., a nucleoside triphosphate transporter capable of transporting unnatural nucleoside triphosphates into the cell, and optionally a CRISPR/Cas9 system to eliminate DNA that has lost the unnatural nucleotide (e.g. E. coli strain YZ3, ML1, or ML2). In some instances, cells further comprise enhanced activity for unnatural nucleic acid uptake. In some cases, cells further comprise enhanced activity for unnatural nucleic acid import.

In some embodiments, Cas9 and an appropriate guide RNA (sgRNA) are encoded on separate plasmids. In some instances, Cas9 and sgRNA are encoded on the same plasmid. In some cases, the nucleic acid molecule encoding Cas9, sgRNA, or a nucleic acid molecule comprising an unnatural nucleotide are located on one or more plasmids. In some instances, Cas9 is encoded on a first plasmid and the sgRNA and the nucleic acid molecule comprising an unnatural nucleotide are encoded on a second plasmid. In some instances, Cas9, sgRNA, and the nucleic acid molecule comprising an unnatural nucleotide are encoded on the same plasmid. In some instances, the nucleic acid molecule comprises two or more unnatural nucleotides. In some instances, Cas9 is incorporated into the genome of the host organism and sgRNAs are encoded on a plasmid or in the genome of the organism.

In some instances, a first plasmid encoding Cas9 and sgRNA and a second plasmid encoding a nucleic acid molecule comprising an unnatural nucleotide are introduced into an engineered microorganism. In some instances, a first plasmid encoding Cas9 and a second plasmid encoding sgRNA and a nucleic acid molecule comprising an unnatural nucleotide are introduced into an engineered microorganism. In some instances, a plasmid encoding Cas9, sgRNA and a nucleic acid molecule comprising an unnatural nucleotide is introduced into an engineered microorganism. In some instances, the nucleic acid molecule comprises two or more unnatural nucleotides.

In some embodiments, a living cell is generated that incorporates within its DNA (plasmid or genome) at least one unnatural nucleic acid molecule comprising at least one unnatural base pair (UBP). In some cases, the at least one unnatural nucleic acid molecule comprises one, two, three, four, or more UBPs. In some instances, the at least one unnatural nucleic acid molecule is a plasmid. In some cases, the at least one unnatural nucleic acid molecule is integrated into the genome of the cell. In some embodiments, the at least on unnatural nucleic acid molecule encodes the unnatural polypeptide or the unnatural protein. In some cases, the at least one unnatural nucleic acid molecule is transcribed to afford the unnatural codon of the mRNA and the unnatural anticodon of the tRNA. In some embodiments, the at least one unnatural nucleic acid molecule is an unnatural DNA molecule.

In some instances, the unnatural base pair includes a pair of unnatural mutually base-pairing nucleotides capable of forming the unnatural base pair under in vivo conditions, when the unnatural mutually base-pairing nucleotides, as their respective triphosphates, are taken up into the cell by action of a nucleotide triphosphate transporter. The cell can be genetically transformed by an expression cassette encoding a nucleotide triphosphate transporter so that the nucleotide triphosphate transporter is expressed and is available to transport the unnatural nucleotides into the cell. The cell can be a prokaryotic or eukaryotic cell, and the pair of unnatural mutually base-pairing nucleotides, as their respective triphosphates, can be a triphosphate of dTPT3 (dTP3TP) and a triphosphate of dNaM (dNaMTP) or dCNMO (dCNMOTP).

In some embodiments, cells are genetically transformed cells with a nucleic acid, e.g., an expression cassette encoding a nucleotide triphosphate transporter capable of transporting such unnatural nucleotides into the cell. A cell can comprise a heterologous nucleoside triphosphate transporter, where the heterologous nucleoside triphosphate transporter can transport natural and unnatural nucleoside triphosphates into the cell.

In some cases, the methods described herein also include contacting a genetically transformed cell with the respective triphosphates, in the presence of potassium phosphate and/or an inhibitor of phosphatases or nucleotidases. During or after such contact, the cell can be placed within a life-supporting medium suitable for growth and replication of the cell. The cell can be maintained in the life-supporting medium so that the respective triphosphate forms of unnatural nucleotides are incorporated into nucleic acids within the cells, and through at least one replication cycle of the cell. The pair of unnatural mutually base-pairing nucleotides as a respective triphosphate, can comprise a triphosphate of dTPT3 or (dTPT3TP) and a triphosphate of dCNMO or dNaM (dCNOM or dNaMTP), the cell can be E. coli, and the dTPT3TP and dNaMTP can be imported into E. coli by the transporter PtNTT2, wherein an E. coli polymerase, such as Pol III or Pol II, can use the unnatural triphosphates to replicate DNA containing a UBP, thereby incorporating unnatural nucleotides and/or unnatural base pairs into cellular nucleic acids within the cellular environment. Additionally, ribonucleotides such as NaMTP and TAT1TP, 5FMTP, and TPT3TP are in some instances imported into E. coli by the transporter PtNTT2. In some instances, the PtNTT2 for importing ribonucleotides is a truncated PtNTT2, where the truncated PtNTT2 has an amino acid sequence that is at least 60%, at least 65%, at least 70%, at least 75%, at least 80%, at least 85%, or at least 90% identical to the amino acid sequence of untruncated PtNTT2. An example of untruncated PtNTT2 (NCBI accession number EEC49227.1, GI:217409295) has the amino acid sequence (SEQ ID NO: 1):

```
    1 MRPYPTIALI SVFLSAATRI SATSSHQASA LPVKKGTHVP
  41 DSPKLSKLYI MAKTKSVSSS FDPPRGGSTV APTTPLATGG
  81 ALRKVRQAVF PIYGNQEVTK FLLIGSIKFF IILALTLTRD
121 TKDTLIVTQC GAEAIAFLKI YGVLPAATAF IALYSKMSNA
161 MGKKMLFYST CIPFFTFFGL FDVFIYPNAE RLHPSLEAVQ
201 AILPGGAASG GMAVLAKIAT HWTSALFYVM AEIYSSVSVG
241 LLFWQFANDV VNVDQAKRFY PLFAQMSGLA PVLAGQYVVR
281 FASKAVNFEA SMHRLTAAVT FAGIMICIFY QLSSSYVERT
321 ESAKPAADNE QSIKPKKKKP KMSMVESGKF LASSQYLRLI
361 AMLVLGYGLS INFTEIMWKS LVKKQYPDPL DYQRFMGNFS
401 SAVGLSTCIV IFFGVHVIRL LGWKVGALAT PGIMAILALP
441 FFACILLGLD SPARLEIAVI FGTIQSLLSK TSKYALFDPT
481 TQMAYIPLDD ESKVKGKAAI DVLGSRIGKS GGSLIQQGLV
521 FVFGNIINAA PVVGVVYYSV LVAWMSAAGR LSGLFQAQTE
561 MDKADKMEAK TNKEK
```

Described herein are compositions and methods comprising the use of three or more unnatural base-pairing nucleotides. Such base pairing nucleotides in some cases enter a cell through use of nucleotide transporters, or through standard nucleic acid transformation methods known in the art (e.g., electroporation, chemical transformation, or other methods). In some cases, a base pairing unnatural nucleotide enters a cell as part of a polynucleotide, such as a plasmid. One or more base pairing unnatural nucleotide which enter a cell as part of a polynucleotide (RNA or DNA) need not themselves be replicated in vivo. For example, a double-stranded DNA plasmid or other nucleic acid comprising a first unnatural deoxyribonucleotide and a second unnatural deoxyribonucleotide with bases configured to form a first unnatural base pair are electroporated into a cell. The cell media is treated with a third unnatural deoxyribonucleotide, a fourth unnatural deoxyribonucleotide with bases configured to form a second unnatural base pair with each other, wherein the first unnatural deoxyribonucleotide's base and the third unnatural deoxyribonucleotide's base form a second unnatural base pair, and wherein the second unnatural deoxyribonucleotide's base and the fourth unnatural deoxyribonucleotide's base form a third unnatural base pair. In some instances, in vivo replication of the originally transformed double-stranded DNA plasmid results in subsequent replicated plasmids comprising the third unnatural deoxyribonucleotide and the fourth unnatural deoxyribonucleotide. Alternatively, or in combination, ribonucleotides variants of the third unnatural deoxyribonucleotide and fourth unnatural deoxyribonucleotide are added to the cell media. These ribonucleotides are in some instances incorporated into RNA, such as mRNA or tRNA. In some instances, the first, second, third, and fourth deoxynucleotides comprise different bases. In some instances, the first, third, and fourth deoxynucleotides comprise different bases. In some instances, the first and third deoxynucleotides comprise the same base.

By practice of the methods of the present disclosure, the person of ordinary skill can obtain a population of a living and propagating cells that has at least one unnatural nucleotide and/or at least one unnatural base pair (UBP) within at least one nucleic acid maintained within at least some of the individual cells, wherein the at least one nucleic acid is stably propagated within the cell, and wherein the cell expresses a nucleotide triphosphate transporter suitable for providing cellular uptake of triphosphate forms of one or more unnatural nucleotides when contacted with (e.g., grown in the presence of) the unnatural nucleotide(s) in a life-supporting medium suitable for growth and replication of the organism.

After transport into the cell by the nucleotide triphosphate transporter, the unnatural base-pairing nucleotides are incorporated into nucleic acids within the cell by cellular machinery, e.g., the cell's own DNA and/or RNA polymerases, a heterologous polymerase, or a polymerase that has been evolved using directed evolution (Chen T, Romesberg F E, FEBS Lett. 2014 Jan. 21; 588(2):219-29; Betz K et al., J Am Chem Soc. 2013 Dec. 11; 135(49):18637-43). The unnatural nucleotides can be incorporated into cellular nucleic acids such as genomic DNA, genomic RNA, mRNA, tRNA, structural RNA, microRNA, and autonomously replicating nucleic acids (e.g., plasmids, viruses, or vectors).

In some cases, genetically engineered cells are generated by introduction of nucleic acids, e.g., heterologous nucleic acids, into cells. In some instances, the nucleic acids being introduced into the cells are in the form of a plasmid. In some cases, the nucleic acids being introduced into the cells are integrated into the genome of the cell. Any cell described herein can be a host cell and can comprise an expression vector. In one embodiment, the host cell is a prokaryotic cell. In another embodiment, the host cell is E. coli. In some embodiments, a cell comprises one or more heterologous polynucleotides. Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, conjugation, particle bombardment and the like. In some instances, the addition of carrier molecules (e.g., bis-benzoimidazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some instances, genetic transformation is obtained using direct transfer of an expression cassette, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the methods described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, DNA encoding a nucleoside triphosphate transporter or polymerase expression cassette and/or vector can be introduced to a cell by any methods including, but not limited to, calcium-mediated transformation, electroporation, microinjection, lipofection, particle bombardment and the like.

In some cases, a cell comprises unnatural nucleoside triphosphates incorporated into one or more nucleic acids within the cell. For example, the cell can be a living cell capable of incorporating at least one unnatural nucleotide within DNA or RNA maintained within the cell. The cell can also incorporate at least one unnatural base pair (UBP) comprising a pair of unnatural mutually base-pairing nucleotides into nucleic acids within the cell under in vivo conditions, wherein the unnatural mutually base-pairing nucleotides, e.g., their respective triphosphates, are taken up into the cell by action of a nucleoside triphosphate transporter, the gene for which is present (e.g., was introduced) into the cell by genetic transformation. For example, upon incorporation into the nucleic acid maintained within the cell, dTPT3 and dCNMO can form a stable unnatural base pair that can be stably propagated by the DNA replication machinery of an organism, e.g., when grown in a life-supporting medium comprising dTPT3TP and dCNMOTP.

In some cases, cells are capable of replicating a nucleic acid containing an unnatural nucleotide. Such methods can include genetically transforming the cell with an expression cassette encoding a nucleoside triphosphate transporter capable of transporting into the cell, as a respective triphosphate, one or more unnatural nucleotides under in vivo conditions. Alternatively, a cell can be employed that has previously been genetically transformed with an expression cassette that can express an encoded nucleoside triphosphate transporter. The methods can also include contacting or exposing the genetically transformed cell to potassium phosphate and the respective triphosphate forms of at least one unnatural nucleotide (for example, two mutually base-pairing nucleotides capable of forming the unnatural base pair (UBP)) in a life-supporting medium suitable for growth and replication of the cell, and maintaining the transformed cell in the life-supporting medium in the presence of the respective triphosphate forms of at least one unnatural nucleotide (for example, two mutually base-pairing nucleotides capable of forming the unnatural base pair (UBP)) under in vivo conditions, through at least one replication cycle of the cell.

In some embodiments, a cell comprises a stably incorporated unnatural nucleic acid. Some embodiments comprise a cell (e.g., as E. coli) that stably incorporates nucleotides other than A, G, T, and C within nucleic acids maintained within the cell. For example, the nucleotides other than A, G, T, and C can be d5SICS, dCNMO, dNaM, and/or dTPT3, which upon incorporation into nucleic acids of the cell, can form a stable unnatural base pair within the nucleic acids. In one aspect, unnatural nucleotides and unnatural base pairs can be stably propagated by the replication apparatus of the organism, when an organism transformed with the gene for the triphosphate transporter, is grown in a life-supporting medium that includes potassium phosphate and the triphosphate forms of d5SICS, dNaM, dCNMO, and/or dTPT3.

In some cases, a cell comprises an expanded genetic alphabet. A cell can comprise a stably incorporated unnatural nucleic acid. In some embodiments, a cell with an expanded genetic alphabet comprises an unnatural nucleic acid that contains an unnatural nucleotide that can pair with another unnatural nucleotide. In some embodiments, a cell with an expanded genetic alphabet comprises an unnatural nucleic acid that is hydrogen bonded to another nucleic acid. In some embodiments, a cell with an expanded genetic alphabet comprises an unnatural nucleic acid that is not hydrogen bonded to another nucleic acid to which it is base paired. In some embodiments, a cell with an expanded genetic alphabet comprises an unnatural nucleic acid that contains an unnatural nucleotide with a nucleobase that base pairs to the nucleobase or another unnatural nucleotide via hydrophobic and/or packing interactions. In some embodiments, a cell with an expanded genetic alphabet comprises an unnatural nucleic acid that base pairs to another nucleic acid via non-hydrogen bonding interactions. A cell with an expanded genetic alphabet can be a cell that can copy a homologous nucleic acid to form a nucleic acid comprising an unnatural nucleic acid. A cell with an expanded genetic alphabet can be a cell comprising an unnatural nucleic acid base paired with another unnatural nucleic acid (unnatural nucleic acid base pair (UBP)).

In some embodiments, cells form unnatural DNA base pairs (UBPs) from the imported unnatural nucleotides under in vivo conditions. In some embodiments, potassium phosphate and/or inhibitors of phosphatase and/or nucleotidase activities can facilitate transport of unnatural nucleotides. The methods include use of a cell that expresses a heterologous nucleoside triphosphate transporter. When such a cell is contacted with one or more nucleoside triphosphates, the nucleoside triphosphates are transported into the cell. The cell can be in the presence of potassium phosphate and/or inhibitors of phosphatases and nucleotidases. Unnatural nucleoside triphosphates can be incorporated into nucleic acids within the cell by the cell's natural machinery (i.e. polymerases) and, for example, mutually base-pair to form unnatural base pairs within the nucleic acids of the cell. In some embodiments, UBPs are formed between DNA and RNA nucleotides bearing unnatural bases.

In some embodiments, a UBP can be incorporated into a cell or population of cells when exposed to unnatural triphosphates. In some embodiments a UBP can be incorporated into a cell or population of cells when substantially consistently exposed to unnatural triphosphates.

In some embodiments, induction of expression of a heterologous gene, e.g., a nucleoside triphosphate transporter (NTT), in a cell can result in slower cell growth and increased unnatural triphosphate uptake compared to the growth and uptake of one or more unnatural triphosphates in a cell without induction of expression of the heterologous gene. Uptake variously comprises transport of nucleotides into a cell, such as through diffusion, osmosis, or via the action of transporters. In some embodiments, induction of expression of a heterologous gene, e.g., an NTT, in a cell can result in increased cell growth and increased unnatural nucleic acid uptake compared to the growth and uptake of a cell without induction of expression of the heterologous gene.

In some embodiments, a UBP is incorporated during a log growth phase. In some embodiments, a UBP is incorporated during a non-log growth phase. In some embodiments, a UBP is incorporated during a substantially linear growth phase. In some embodiments a UBP is stably incorporated into a cell or population of cells after growth for a time period. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 50 or more duplications. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, or 24 hours of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days of growth. For example, a UBP can be stably incorporated into a cell or population of cells after growth for at least about 1, 2, 3, 4, 5, 6, 7, 8, 9, 10, 11, or 12 months of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50 years of growth.

In some embodiments, a cell further utilizes an RNA polymerase to generate an mRNA which contains one or more unnatural nucleotides. In some instances, a cell further utilizes a polymerase to generate a tRNA which contains an anticodon that comprises one or more unnatural nucleotides. In some instances, the tRNA is charged with an unnatural amino acid. In some instances, the unnatural anticodon of the tRNA pairs with the unnatural codon of an mRNA during translation to synthesis an unnatural polypeptide or an unnatural protein that contains at least one unnatural amino acid.

Natural and Unnatural Amino Acids

As used herein, an amino acid residue can refer to a molecule containing both an amino group and a carboxyl group. Suitable amino acids include, without limitation, both the D- and L-isomers of the naturally-occurring amino acids, as well as non-naturally occurring amino acids prepared by organic synthesis or any other methods. The term amino acid, as used herein, includes, without limitation, u-amino acids, natural amino acids, non-natural amino acids, and amino acid analogs.

The term “a-amino acid” can refer to a molecule containing both an amino group and a carboxyl group bound to a carbon which is designated the a-carbon. For example:

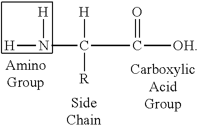

The term “-amino acid” can refer to a molecule containing both an amino group and a carboxyl group in a 3 configuration.

“Naturally occurring amino acid” can refer to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | | Side-chain | |
|---|---|---|---|---|---|
| | Letter | Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive (10%) | −3.2 |
| | | | | neutral (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” can be glycine, alanine, proline, and analogs thereof. “Large hydrophobic amino acids” can be valine, leucine, isoleucine, phenylalanine, methionine, tryptophan, and analogs thereof “Polar amino acids” can be serine, threonine, asparagine, glutamine, cysteine, tyrosine, and analogs thereof. “Charged amino acids” can be lysine, arginine, histidine, aspartate, glutamate, and analogs thereof.

An “amino acid analog” can be a molecule which is structurally similar to an amino acid and which can be substituted for an amino acid in the formation of a peptidomimetic macrocycle Amino acid analogs include, without limitation, R-amino acids and amino acids where the amino or carboxy group is substituted by a similarly reactive group (e.g., substitution of the primary amine with a secondary or tertiary amine, or substitution of the carboxy group with an ester).

A non-cannonical amino acid (ncAA) or “non-natural amino acid” can be an amino acid which is not one of the twenty amino acids commonly found in peptides synthesized in nature, and known by the one letter abbreviations A, R, N, C, D, Q, E, G, H, I, L, K, M, F, P, S, T, W, Y and V. In some instances, non-natural amino acids are a subset of non-canonical amino acids.

Amino acid analogs can include R-amino acid analogs. Examples of 3-amino acid analogs include, but are not limited to, the following: cyclic-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahy dro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-No-benzyloxycarbonyl-p-homolysine; NO-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-p-phenylalanine; L-β-homoaspartic acid y-t-butyl ester; L-β-homoglutamic acid δ-t-butyl ester; L-NO-p-homolysine; N6-trityl-L-β-homoglutamine; No-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs can include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,ω-diaminopropionic acid; (N-β-1-(4,4-dimethyl-2,6-dioxocyclohex-1-ylidene)ethyl)-D-α,ω-diaminopropionic acid; (N-β-1-(4,4-dimethyl-2,6-dioxocyclohex-1-ylidene)ethyl)-L-α,ω-diaminopropionic acid; (N-β-4-methyltrityl)-L-α,β-diaminopropionic acid; (N-β-allyloxycarbonyl)-L-α,ω-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs can include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-omithine; No-nitro-D-arginine; NO-nitro-L-arginine; α-methyl-omithine; 2,6-diaminoheptanedioic acid; L-ornithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; No-nitro-D-arginine; and No-nitro-L-arginine.

Amino acid analogs can include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; y-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-p-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs can include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs can include analogs of phenylalanine and tyrosine. Examples of amino acid analogs of phenylalanine and tyrosine include J3-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs can include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs can include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs can include analogs of tryptophan. Examples of amino acid analogs of tryptophan include, but are not limited to, the following: α-methyl-tryptophan; j-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

Amino acid analogs can be racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a j-amino acid analog is protected, e.g., as its ester derivative. In some cases, the salt of the amino acid analog is used.

In some embodiments, an unnatural amino acid is an unnatural amino acid described in Liu C. C., Schultz, P. G. Annu. Rev. Biochem. 2010, 79, 413. In some embodiments, an unnatural amino acid comprises N6(2-azidoethoxy)-carbonyl-L-lysine.

In some embodiments, an amino acid residue described herein (e.g., within a protein) is mutated to an unnatural amino acid prior to binding to a conjugating moiety. In some cases, the mutation to an unnatural amino acid prevents or minimizes a self-antigen response of the immune system. As used herein, the term “unnatural amino acid” refers to an amino acid other than the 20 amino acids that occur naturally in protein. Non-limiting examples of unnatural amino acids include: p-acetyl-L-phenylalanine, p-iodo-L-phenylalanine, p-methoxyphenylalanine, p-methyl-L-tyrosine, p-propargyloxyphenylalanine, p-propargyl-phenylalanine, L-3-(2-naphthyl)alanine, 3-methyl-phenylalanine, p-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-azido-L-phenylalanine p-azido-phenylalanine, p-benzoyl-L-phenylalanine, p-Boronophenylalanine, p-propargyltyrosine, L-phosphoserine, phosphonoserine, phosphonotyrosine, p-bromophenylalanine, selenocysteine, p-amino-L-phenylalanine, isopropyl-L-phenylalanine, N6-(propargyloxy)-carbonyl-L-lysine (PrK), azido-lysine (N6-azidoethoxy-carbonyl-L-lysine, AzK), N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α, α disubstituted amino acid; a β-amino acid; a cyclic amino acid other than proline or histidine, and an aromatic amino acid other than phenylalanine, tyrosine or tryptophan.

In some embodiments, the unnatural amino acid comprises a selective reactive group, or a reactive group for site-selective labeling of a target protein or polypeptide. In some instances, the chemistry is a biorthogonal reaction (e.g., biocompatible and selective reactions). In some cases, the chemistry is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the unnatural amino acid comprises a photoreactive group, which crosslinks, upon irradiation with, e.g., UV. In some embodiments, the unnatural amino acid comprises a photo-caged amino acid. In some instances, the unnatural amino acid is a para-substituted, meta-substituted, or an ortho-substituted amino acid derivative.

In some instances, the unnatural amino acid comprises p-acetyl-L-phenylalanine, p-azidomethyl-L-phenylalanine (pAMF), p-iodo-L-phenylalanine, O-methyl-L-tyrosine, p-methoxyphenylalanine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-bromophenylalanine, p-amino-L-phenylalanine, or isopropyl-L-phenylalanine.

In some cases, the unnatural amino acid is 3-aminotyrosine, 3-nitrotyrosine, 3,4-dihydroxy-phenylalanine, or 3-iodotyrosine. In some cases, the unnatural amino acid is phenylselenocysteine. In some instances, the unnatural amino acid is a benzophenone, ketone, iodide, methoxy, acetyl, benzoyl, or azide containing phenylalanine derivative. In some instances, the unnatural amino acid is a benzophenone, ketone, iodide, methoxy, acetyl, benzoyl, or azide containing lysine derivative. In some instances, the unnatural amino acid comprises an aromatic side chain. In some instances, the unnatural amino acid does not comprise an aromatic side chain. In some instances, the unnatural amino acid comprises an azido group. In some instances, the unnatural amino acid comprises a Michael-acceptor group. In some instances, Michael-acceptor groups comprise an unsaturated moiety capable of forming a covalent bond through a 1,2-addition reaction. In some instances, Michael-acceptor groups comprise electron-deficient alkenes or alkynes. In some instances, Michael-acceptor groups include but are not limited to alpha,beta unsaturated: ketones, aldehydes, sulfoxides, sulfones, nitriles, imines, or aromatics. In some instances, the unnatural amino acid is dehydroalanine. In some instances, the unnatural amino acid comprises an aldehyde or ketone group. In some instances, the unnatural amino acid is a lysine derivative comprising an aldehyde or ketone group. In some instances, the unnatural amino acid is a lysine derivative comprising one or more O, N, Se, or S atoms at the beta, gamma, or delta position. In some instances, the unnatural amino acid is a lysine derivative comprising 0, N, Se, or S atoms at the gamma position. In some instances, the unnatural amino acid is a lysine derivative wherein the epsilon N atom is replaced with an oxygen atom. In some instances, the unnatural amino acid is a lysine derivative that is not naturally-occurring post-translationally modified lysine.

In some instances, the unnatural amino acid is an amino acid comprising a side chain, wherein the sixth atom from the alpha position comprises a carbonyl group. In some instances, the unnatural amino acid is an amino acid comprising a side chain, wherein the sixth atom from the alpha position comprises a carbonyl group, and the fifth atom from the alpha position is nitrogen. In some instances, the unnatural amino acid is an amino acid comprising a side chain, wherein the seventh atom from the alpha position is an oxygen atom.

In some instances, the unnatural amino acid is a serine derivative comprising selenium. In some instances, the unnatural amino acid is selenoserine (2-amino-3-hydroselenopropanoic acid). In some instances, the unnatural amino acid is 2-amino-3-((2-((3-(benzyloxy)-3-oxopropyl)amino)ethyl)selanyl)propanoic acid. In some instances, the unnatural amino acid is 2-amino-3-(phenylselanyl)propanoic acid. In some instances, the unnatural amino acid comprises selenium, wherein oxidation of the selenium results in the formation of an unnatural amino acid comprising an alkene.

In some instances, the unnatural amino acid comprises a cyclooctynyl group. In some instances, the unnatural amino acid comprises a transcycloctenyl group. In some instances, the unnatural amino acid comprises a norbornenyl group. In some instances, the unnatural amino acid comprises a cyclopropenyl group. In some instances, the unnatural amino acid comprises a diazirine group. In some instances, the unnatural amino acid comprises a tetrazine group.

In some instances, the unnatural amino acid is a lysine derivative, wherein the side-chain nitrogen is carbamylated. In some instances, the unnatural amino acid is a lysine derivative, wherein the side-chain nitrogen is acylated. In some instances, the unnatural amino acid is 2-amino-6-{[(tert-butoxy)carbonyl]amino}hexanoic acid. In some instances, the unnatural amino acid is 2-amino-6-{[(tert-butoxy)carbonyl]amino}hexanoic acid. In some instances, the unnatural amino acid is N6-Boc-N6-methyllysine. In some instances, the unnatural amino acid is N6-acetyllysine. In some instances, the unnatural amino acid is pyrrolysine. In some instances, the unnatural amino acid is N6-trifluoroacetyllysine. In some instances, the unnatural amino acid is 2-amino-6-{[(benzyloxy)carbonyl]amino}hexanoic acid. In some instances, the unnatural amino acid is 2-amino-6-{[(p-iodobenzyloxy)carbonyl]amino}hexanoic acid. In some instances, the unnatural amino acid is 2-amino-6-{[(p-nitrobenzyloxy)carbonyl]amino}hexanoic acid. In some instances, the unnatural amino acid is N6-prolyllysine. In some instances, the unnatural amino acid is 2-amino-6-{[(cyclopentyloxy)carbonyl]amino}hexanoic acid. In some instances, the unnatural amino acid is N6-(cyclopentanecarbonyl)lysine. In some instances, the unnatural amino acid is N6-(tetrahydrofuran-2-carbonyl)lysine. In some instances, the unnatural amino acid is N6-(3-ethynyltetrahy drofuran-2-carbonyl)lysine. In some instances, the unnatural amino acid is N6-((prop-2-yn-1-yloxy)carbonyl)lysine. In some instances, the unnatural amino acid is 2-amino-6-{[(2-azidocyclopentyloxy)carbonyl]amino}hexanoic acid. In some instances, the unnatural amino acid is N6-((2-azidoethoxy)carbonyl)lysine. In some instances, the unnatural amino acid is 2-amino-6-{[(2-nitrobenzyloxy)carbonyl]amino}hexanoic acid. In some instances, the unnatural amino acid is 2-amino-6-{[(2-cyclooctynyloxy)carbonyl]amino}hexanoic acid. In some instances, the unnatural amino acid is N6-(2-aminobut-3-ynoyl)lysine. In some instances, the unnatural amino acid is 2-amino-6-((2-aminobut-3-ynoyl)oxy)hexanoic acid. In some instances, the unnatural amino acid is N6-(allyloxy carbonyl)lysine. In some instances, the unnatural amino acid is N6-(butenyl-4-oxycarbonyl)lysine. In some instances, the unnatural amino acid is N6-(pentenyl-5-oxy carbonyl)lysine. In some instances, the unnatural amino acid is N6-((but-3-yn-1-yloxy)carbonyl)-lysine. In some instances, the unnatural amino acid is N6-((pent-4-yn-1-yloxy)carbonyl)-lysine. In some instances, the unnatural amino acid is N6-(thiazolidine-4-carbonyl)lysine. In some instances, the unnatural amino acid is 2-amino-8-oxononanoic acid. In some instances, the unnatural amino acid is 2-amino-8-oxooctanoic acid. In some instances, the unnatural amino acid is N6-(2-oxoacetyl)lysine. In some instances, the unnatural amino acid is N6-(((2-azidobenzyl)oxy)carbonyl)-L-lysine. In some instances, the unnatural amino acid is N6-(((3-azidobenzyl)oxy)carbonyl)-L-lysine. In some instances, the unnatural amino acid is N6-(((4-azidobenzyl)oxy)carbonyl)-L-lysine.

In some instances, the unnatural amino acid is N6-propionyllysine. In some instances, the unnatural amino acid is N6-butyryllysine, In some instances, the unnatural amino acid is N6-(but-2-enoyl)lysine, In some instances, the unnatural amino acid is N6-((bicyclo[2.2.1]hept-5-en-2-yloxy)carbonyl)lysine. In some instances, the unnatural amino acid is N6-((spiro[2.3]hex-1-en-5-ylmethoxy)carbonyl)lysine. In some instances, the unnatural amino acid is N6-(((4-(1-(trifluoromethyl)cycloprop-2-en-1-yl)benzyl)oxy)carbonyl)lysine. In some instances, the unnatural amino acid is N6-((bicyclo[2.2.1]hept-5-en-2-ylmethoxy)carbonyl)lysine. In some instances, the unnatural amino acid is cysteinyllysine. In some instances, the unnatural amino acid is N6-((1-(6-nitrobenzo[d][1,3]dioxol-5-yl)ethoxy)carbonyl)lysine. In some instances, the unnatural amino acid is N6-((2-(3-methyl-3H-diazirin-3-yl)ethoxy)carbonyl)lysine. In some instances, the unnatural amino acid is N6-((3-(3-methyl-3H-diazirin-3-yl)propoxy)carbonyl)lysine. In some instances, the unnatural amino acid is N6-((meta nitrobenyloxy)N6-methylcarbonyl)lysine. In some instances, the unnatural amino acid is N6-((bicyclo[6.1.0]non-4-yn-9-ylmethoxy)carbonyl)-lysine. In some instances, the unnatural amino acid is N6-((cyclohept-3-en-1-yloxy)carbonyl)-L-lysine.

In some instances, the unnatural amino acid is 2-amino-3-(((((benzyloxy)carbonyl)amino)methyl)selanyl)propanoic acid. In some embodiments, the unnatural amino acid is incorporated into an unnatural polypeptide or an unnatural protein by a repurposed amber, opal, or ochre stop codon. In some embodiments, the unnatural amino acid is incorporated into an unnatural polypeptide or an unnatural protein by a 4-base codon. In some embodiments, the unnatural amino acid is incorporated into the protein by a repurposed rare sense codon.

In some embodiments, the unnatural amino acid is incorporated into an unnatural polypeptide or an unnatural protein by an unnatural codon comprising an unnatural nucleotide.

In some instances, incorporation of the unnatural amino acid into a protein is mediated by an orthogonal, modified synthetase/tRNA pair. Such orthogonal pairs comprise a natural or mutated synthetase that is capable of charging the unnatural tRNA with a specific unnatural amino acid, often while minimizing charging of a) other endogenous amino acids or alternate unnnatural amino acids onto the unnatural tRNA and b) any other (including endogenous) tRNAs. Such orthogonal pairs comprise tRNAs that are capable of being charged by the synthetase, while avoiding being charged with other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid can be incorporated into an unnatural polypeptide or an unnatural protein by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, Methanococcus jannaschii (M. jannaschii) TyrRS variant pAzFRS (MjpAzFRS), E. coli TyrRS (Ec-Tyr)/B. stearothermophilus tRNAcuA pairs, E. coli LeuRS (Ec-Leu)/B. stearothermophilus tRNAcuA pairs, and pyrrolysyl-tRNA pairs. In some instances, the unnatural amino acid is incorporated into an unnatural polypeptide or an unnatural protein by a Mj-TyrRS/tRNA pair. Exemplary unnatural amino acids (UAAs) that can be incorporated by a Mj-TyrRS/tRNA pair include, but are not limited to, para-substituted phenylalanine derivatives such as p-Azido-L-Phenylalanine (pAzF), N6-(((2-azidobenzyl)oxy)carbonyl)-L-lysine, N6-(((3-azidobenzyl)oxy)carbonyl)-L-lysine, N6-(((4-azidobenzyl)oxy)carbonyl)-L-lysine, p-aminophenylalanine andp-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronopheylalanine; and o-nitrobenzyltyrosine.

In some instances, the unnatural amino acid can be incorporated into an unnatural polypeptide or an unnatural protein by an Ec-Tyr/tRNAcuA or an Ec-Leu/tRNAcuA pair. Exemplary UAAs that can be incorporated by an Ec-Tyr/tRNAcuA or an Ec-Leu/tRNAcuA pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid can be incorporated into an unnatural polypeptide or an unnatural protein by a pyrrolysyl-tRNA pair. In some cases, the PylRS can be obtained from an archaebacterial species, e.g., from a methanogenic archaebacterium. In some cases, the PylRS can be obtained from Methanosarcina barkeri, Methanosarcina mazei, or Methanosarcina acetivorans. In some cases, the PylRS can be a chimeric PylRS. Exemplary UAAs that can be incorporated by a pyrrolysyl-tRNA pair include, but are not limited to, amide and carbamate substituted lysines such as N6-(2-azidoethoxy)-carbonyl-L-lysine (AzK), N6-(((2-azidobenzyl)oxy)carbonyl)-L-lysine, N6-(((3-azidobenzyl)oxy)carbonyl)-L-lysine, Nδ-(((4-azidobenzyl)oxy)carbonyl)-L-lysine, 2-amino-6-((R)-tetrahydrofuran-2-carboxamido)hexanoic acid, N-ε-_{D}-prolyl-_{L}-lysine, and N-ε-cyclopentyloxycarbonyl-L-lysine; N-ε-Acryloyl-_{L}-lysine; N-ε-[(1-(6-nitrobenzo[d][1,3]dioxol-5-yl)ethoxy)carbonyl]-L-lysine; and N-ε-(1-methylcyclopro-2-enecarboxamido)lysine.

In some case, the compositions and methods as described herein comprise using at least two tRNA synthetases to incorporate at least two unnatural amino acids into the unnatural polypeptide or unnatural protein. In some cases, the at least two tRNA synthetases can be same or different. In cases, the at least two unnatural amino acids can be the same or different. In some instances, the at least two unnatural amino acids being incorporated into the unnatural polypeptide are different. In some instances, the at least two different unnatural amino acids can be incorporated into the unnatural polypeptide or unnatural protein in a site-specific manner.

In some instances, an unnatural amino acid can be incorporated into an unnatural polypeptide or unnatural protein described herein by a synthetase disclosed in U.S. Pat. Nos. 9,988,619 and 9,938,516. Exemplary UAAs that can be incorporated by such synthetases include para-methylazido-L-phenylalanine, aralkyl, heterocyclyl, heteroaralkyl unnatural amino acids, and others. In some embodiments, such UAAs comprise pyridyl, pyrazinyl, pyrazolyl, triazolyl, oxazolyl, thiazolyl, thiophenyl, or other heterocycle. Such amino acids in some embodiments comprise azides, tetrazines, or other chemical group capable of conjugation to a coupling partner, such as a water soluble moiety. In some embodiments, such synthetases are expressed and used to incorporate UAAs into proteins in vivo. In some embodiments, such synthetases are used to incorporate UAAs into proteins using a cell-free translation system.

In some instances, an unnatural amino acid can be incorporated into an unnatural polypeptide or unnatural protein described herein by a naturally occurring synthetase. In some embodiments, an unnatural amino acid is incorporated into an unnatural polypeptide or unnatural protein by an organism that is auxotrophic for one or more amino acids. In some embodiments, synthetases corresponding to the auxotrophic amino acid are capable of charging the corresponding tRNA with an unnatural amino acid. In some embodiments, the unnatural amino acid is selenocysteine, or a derivative thereof. In some embodiments, the unnatural amino acid is selenomethionine, or a derivative thereof. In some embodiments, the unnatural amino acid is an aromatic amino acid, wherein the aromatic amino acid comprises an aryl halide, such as an iodide. In embodiments, the unnatural amino acid is structurally similar to the auxotrophic amino acid.

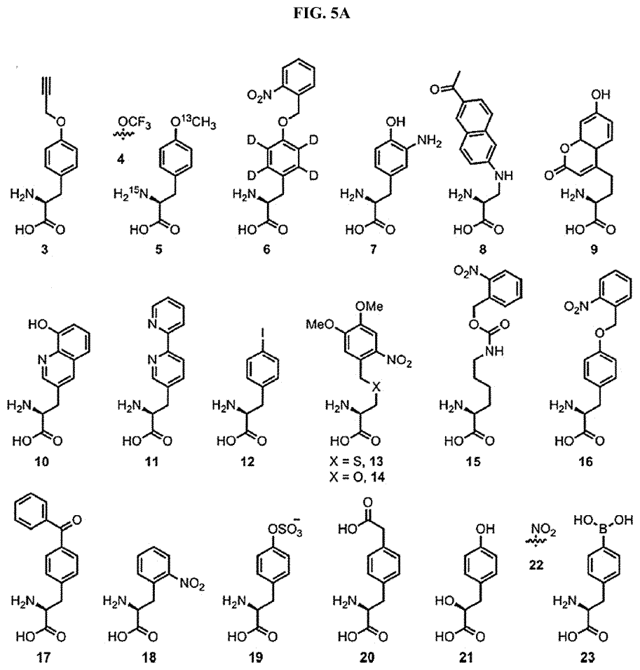

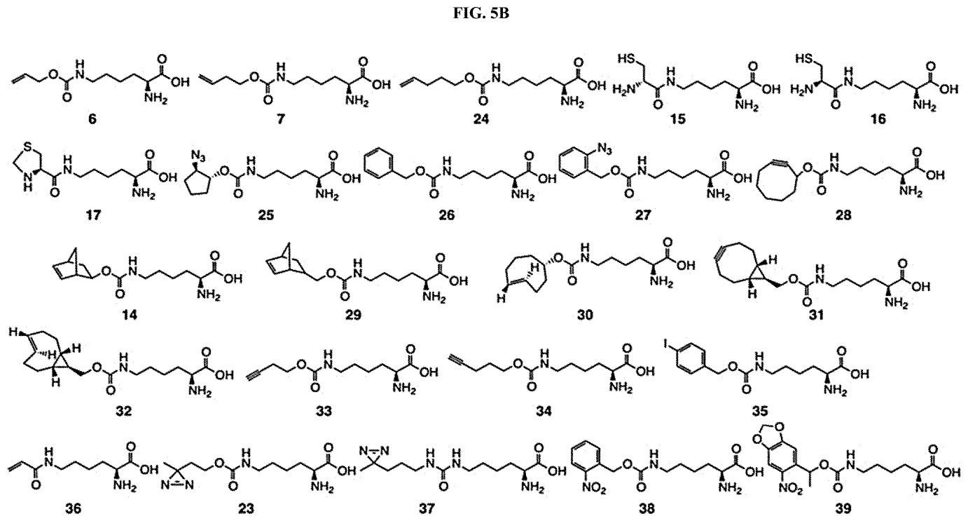

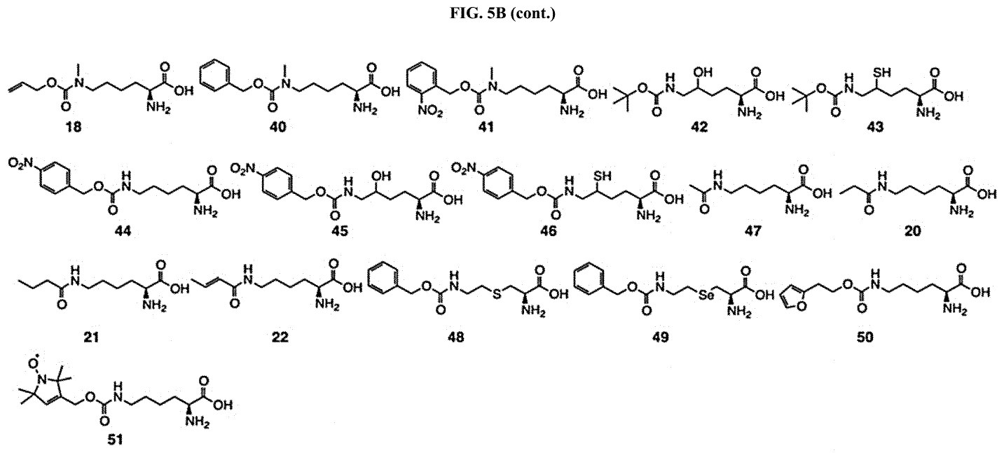

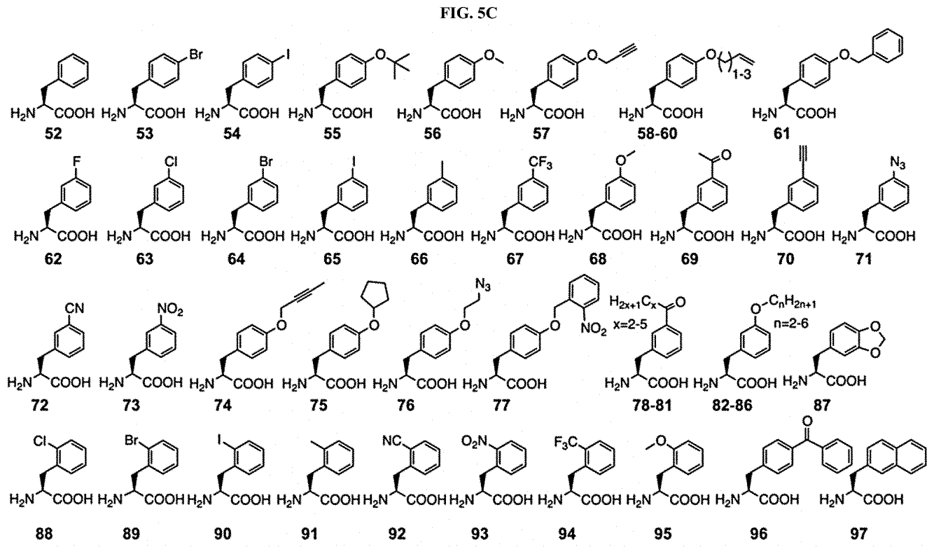

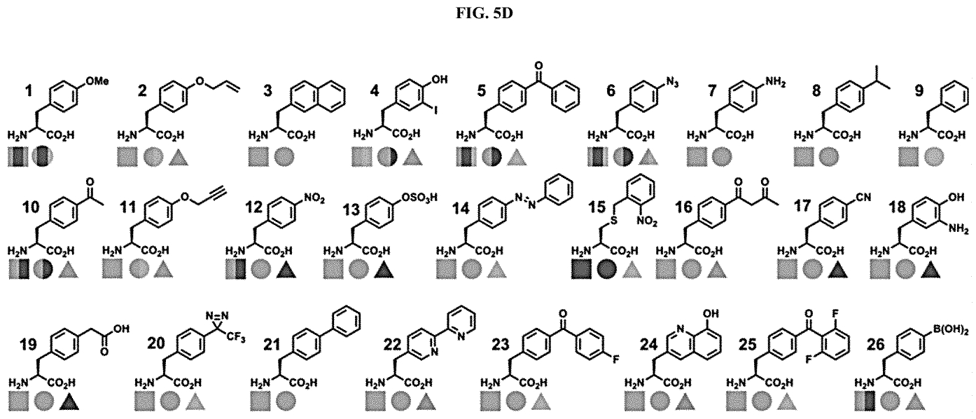

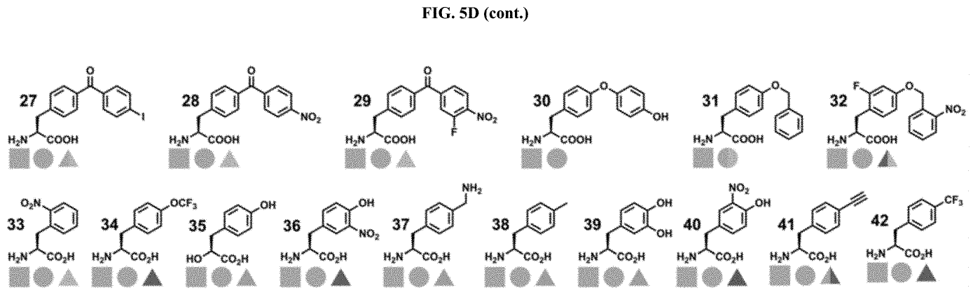

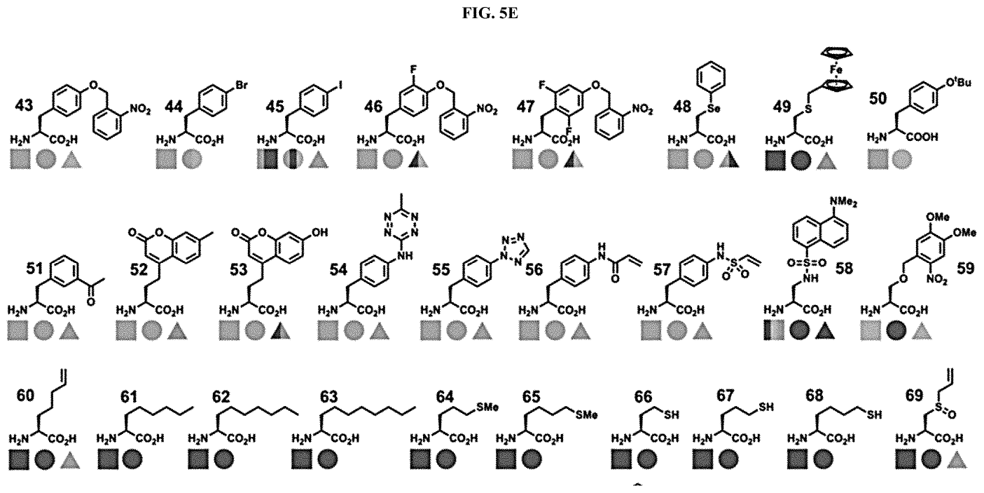

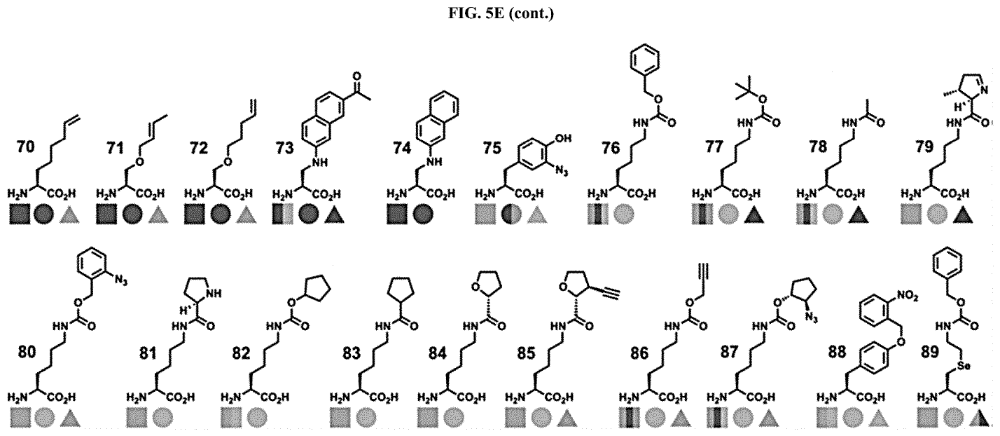

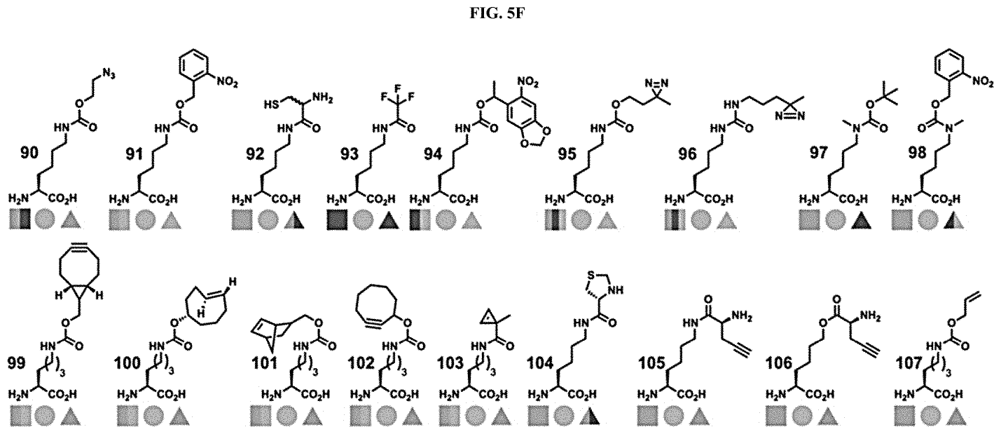

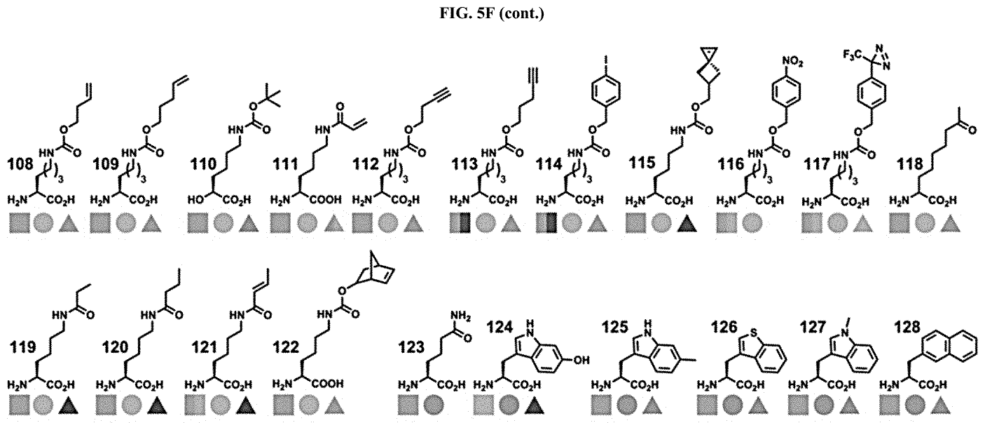

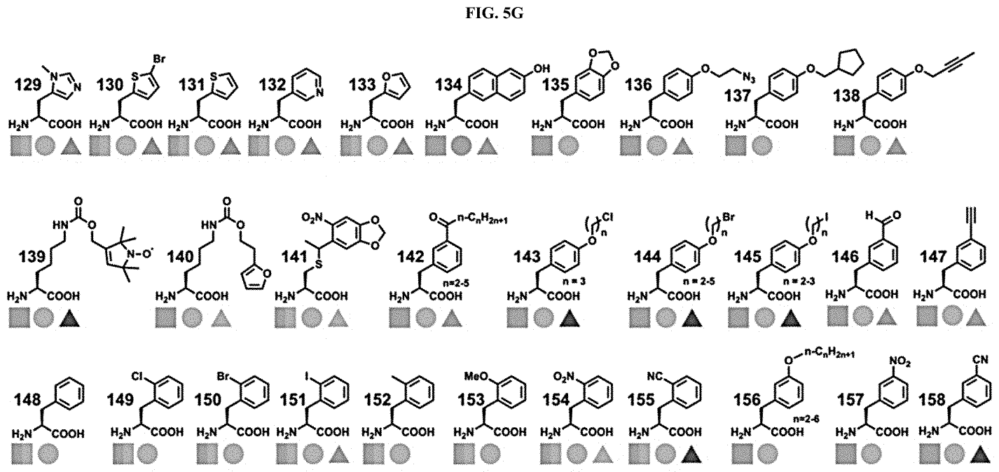

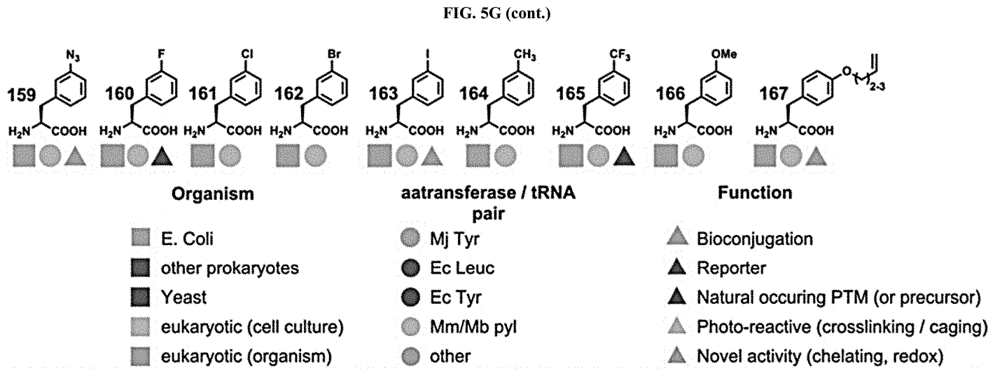

In some instances, the unnatural amino acid comprises an unnatural amino acid illustrated in FIG. 5a.

In some instances, the unnatural amino acid comprises a lysine or phenylalanine derivative or analogue. In some instances, the unnatural amino acid comprises a lysine derivative or a lysine analogue. In some instances, the unnatural amino acid comprises a pyrrolysine (Pyl). In some instances, the unnatural amino acid comprises a phenylalanine derivative or a phenylalanine analogue. In some instances, the unnatural amino acid is an unnatural amino acid described in Wan, et al., “Pyrrolysyl-tRNA synthetase: an ordinary enzyme but an outstanding genetic code expansion tool,” Biocheim Biophys Aceta 1844(6): 1059-4070 (2014). In some instances, the unnatural amino acid comprises an unnatural amino acid illustrated in FIG. 5B and FIG. 5C.

In some embodiments, the unnatural amino acid comprises an unnatural amino acid illustrated in FIG. 5D-FIG. 5G (adopted from Table 1 of Dumas et al., Chemical Science 2015, 6, 50-69).

In some embodiments, an unnatural amino acid incorporated into a protein described herein is disclosed in U.S. Pat. Nos. 9,840,493; 9,682,934; US 2017/0260137; U.S. Pat. No. 9,938,516; or US 2018/0086734. Exemplary UAAs that can be incorporated by such synthetases include para-methylazido-L-phenylalanine, aralkyl, heterocyclyl, and heteroaralkyl, and lysine derivative unnatural amino acids. In some embodiments, such UAAs comprise pyridyl, pyrazinyl, pyrazolyl, triazolyl, oxazolyl, thiazolyl, thiophenyl, or other heterocycle. Such amino acids in some embodiments comprise azides, tetrazines, or other chemical group capable of conjugation to a coupling partner, such as a water soluble moiety. In some embodiments, a UAA comprises an azide attached to an aromatic moiety via an alkyl linker. In some embodiments, an alkyl linker is a C₁-C₁₀ linker. In some embodiments, a UAA comprises a tetrazine attached to an aromatic moiety via an alkyl linker. In some embodiments, a UAA comprises a tetrazine attached to an aromatic moiety via an amino group. In some embodiments, a UAA comprises a tetrazine attached to an aromatic moiety via an alkylamino group. In some embodiments, a UAA comprises an azide attached to the terminal nitrogen (e.g., N6 of a lysine derivative, or N5, N4, or N3 of a derivative comprising a shorter alkyl side chain) of an amino acid side chain via an alkyl chain. In some embodiments, a UAA comprises a tetrazine attached to the terminal nitrogen of an amino acid side chain via an alkyl chain. In some embodiments, a UAA comprises an azide or tetrazine attached to an amide via an alkyl linker. In some embodiments, the UAA is an azide or tetrazine-containing carbamate or amide of 3-aminoalanine, serine, lysine, or derivative thereof. In some embodiments, such UAAs are incorporated into proteins in vivo. In some embodiments, such UAAs are incorporated into proteins in a cell-free system.

### Cell Types

In some embodiments, many types of cells/microorganisms are used, e.g., for transforming or genetically engineering. In some embodiments, a cell is a prokaryotic or eukaryotic cell. In some cases, the cell is a microorganism such as a bacterial cell, fungal cell, yeast, or unicellular protozoan. In other cases, the cell is a eukaryotic cell, such as a cultured animal, plant, or human cell. In additional cases, the cell is present in an organism such as a plant or animal.

In some embodiments, an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba). In some embodiments, an engineered microorganism is a fungus. In some embodiments, an engineered organism is a yeast.

Any suitable yeast may be selected as a host microorganism, engineered microorganism, genetically modified organism or source for a heterologous or modified polynucleotide. Yeast include, but are not limited to, Yarrowia yeast (e.g., Y. lipolytica (formerly classified as Candida lipolytica)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a suitable yeast is of the genus Arachniotus, Aspergillus, Aureobasidium, Auxarthron, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pachysolen, Penicillium, Pichia, Rhodosporidium, Rhodotorula, Rhodotorula, Saccharomyces, Schizosaccharomyces, Scopulariopsis, Sepedonium, Trichosporon, or Yarrowia. In some embodiments, a suitable yeast is of the species Arachniotus flavoluteus, Aspergillus flavus, Aspergillus fumigatus, Aspergillus niger, Aureobasidium pullulans, Auxarthron thaxteri, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporuim keratinophilum, Coccidiodes immitis, Cryptococcus albidus var. diffluens, Cryptococcus laurentii, Cryptococcus neofomans, Debaryomyces hansenii, Gymnoascus dugwayensis, Hansenula anomala, Histoplasma capsulatum, Issatchenkia occidentalis, Isstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutaneum, Trichosporon pullans, Yarrowia lipolytica, or Yarrowia lipolytica (formerly classified as Candida lipolytica). In some embodiments, a yeast is a Y. lipolytica strain that includes, but is not limited to, ATCC20362, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a Candida species (i.e., Candida spp.) yeast. Any suitable Candida species can be used and/or genetically modified for production of a fatty dicarboxylic acid (e.g., octanedioic acid, decanedioic acid, dodecanedioic acid, tetradecanedioic acid, hexadecanedioic acid, octadecanedioic acid, eicosanedioic acid). In some embodiments, suitable Candida species include, but are not limited to Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii and any other Candida spp. yeast described herein. Non-limiting examples of Candida spp. strains include, but are not limited to, sAA001 (ATCC20336), sAA002 (ATCC20913), sAA003 (ATCC20962), sAA496 (US2012/0077252), sAA106 (US2012/0077252), SU-2 (ura3-/ura3-), H5343 (beta oxidation blocked; U.S. Pat. No. 5,648,247) strains. Any suitable strains from Candida spp. yeast may be utilized as parental strains for genetic modification.

Yeast genera, species and strains are often so closely related in genetic content that they can be difficult to distinguish, classify and/or name. In some cases strains of C. lipolytica and Y. lipolytica can be difficult to distinguish, classify and/or name and can be, in some cases, considered the same organism. In some cases, various strains of C. tropicalis and C. viswanathii can be difficult to distinguish, classify and/or name (for example see Arie et. al., J. Gen. Appl. Microbiol., 46, 257-262 (2000). Some C. tropicalis and C. viswanathii strains obtained from ATCC as well as from other commercial or academic sources can be considered equivalent and equally suitable for the embodiments described herein. In some embodiments, some parental strains of C. tropicalis and C. viswanathii are considered to differ in name only.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable prokaryote may be selected as a host microorganism, engineered microorganism or source for a heterologous polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stbl2, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C₁₂₇, BHK, Per-C₆, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea llavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata).

Microorganisms or cells used as host organisms or source for a heterologous polynucleotide are commercially available. Microorganisms and cells described herein, and other suitable microorganisms and cells are available, for example, from Invitrogen Corporation, (Carlsbad, Calif.), American Type Culture Collection (Manassas, Va.), and Agricultural Research Culture Collection (NRRL; Peoria, Ill.). Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Polymerases

A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases.

The ability to improve specificity, processivity, or other features of polymerases unnatural nucleic acids would be highly desirable in a variety of contexts where, e.g., unnatural nucleic acid incorporation is desired, including amplification, sequencing, labeling, detection, cloning, and many others

In some instances, disclosed herein includes polymerases that incorporate unnatural nucleic acids into a growing template copy, e.g., during DNA amplification. In some embodiments, polymerases can be modified such that the active site of the polymerase is modified to reduce steric entry inhibition of the unnatural nucleic acid into the active site. In some embodiments, polymerases can be modified to provide complementarity with one or more unnatural features of the unnatural nucleic acids. Such polymerases can be expressed or engineered in cells for stably incorporating a UBP into the cells. Accordingly, the present disclosure includes compositions that include a heterologous or recombinant polymerase and methods of use thereof.

Polymerases can be modified using methods pertaining to protein engineering. For example, molecular modeling can be carried out based on crystal structures to identify the locations of the polymerases where mutations can be made to modify a target activity. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of polymerases can be used in methods or compositions set forth herein including, for example, protein-based enzymes isolated from biological systems and functional variants thereof. Reference to a particular polymerase, such as those exemplified below, will be understood to include functional variants thereof unless indicated otherwise. In some embodiments, a polymerase is a wild type polymerase. In some embodiments, a polymerase is a modified, or mutant, polymerase.

Polymerases, with features for improving entry of unnatural nucleic acids into active site regions and for coordinating with unnatural nucleotides in the active site region, can also be used. In some embodiments, a modified polymerase has a modified nucleotide binding site.

In some embodiments, a modified polymerase has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid. In some embodiments, a modified or wild type polymerase has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified sugar. In some embodiments, a modified or wild type polymerase has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward a nucleic acid comprising a triphosphate and/or the unnatural nucleic acid without the triphosphate. For example, a modified or wild type polymerase can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid with a diphosphate or monophosphate, or no phosphate, or a combination thereof.

In some embodiments, a modified or wild type polymerase has a relaxed specificity for an unnatural nucleic acid. In some embodiments, a modified or wild type polymerase has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid.

Absence of exonuclease activity can be a wild type characteristic or a characteristic imparted by a variant or engineered polymerase. For example, an exo minus Klenow fragment is a mutated version of Klenow fragment that lacks 3′ to 5′ proofreading exonuclease activity.

The methods of the present disclosure can be used to expand the substrate range of any DNA polymerase which lacks an intrinsic 3 to 5′ exonuclease proofreading activity or where a 3 to 5′ exonuclease proofreading activity has been disabled, e.g. through mutation. Examples of DNA polymerases include polA, polB (see e.g. Parrel & Loeb, Nature Struc Biol 2001) polC, polD, polY, polX and reverse transcriptases (RT) but preferably are processive, high-fidelity polymerases (PCT/GB2004/004643). In some embodiments a modified or wild type polymerase substantially lacks 3′ to 5′ proofreading exonuclease activity. In some embodiments a modified or wild type polymerase substantially lacks 3′ to 5′ proofreading exonuclease activity for an unnatural nucleic acid. In some embodiments, a modified or wild type polymerase has a 3′ to 5′ proofreading exonuclease activity. In some embodiments, a modified or wild type polymerase has a 3′ to 5′ proofreading exonuclease activity for a natural nucleic acid and substantially lacks 3′ to 5′ proofreading exonuclease activity for an unnatural nucleic acid.

In some embodiments, a modified polymerase has a 3′ to 5′ proofreading exonuclease activity that is at least about 60%, 70%, 80%, 90%, 95%, 97%, 98%, 99%, 99.5%, 99.99% the proofreading exonuclease activity of the wild type polymerase. In some embodiments, a modified polymerase has a 3′ to 5′ proofreading exonuclease activity for an unnatural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for an unnatural nucleic acid and a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to the natural nucleic acid.

In some embodiments, polymerases are characterized according to their rate of dissociation from nucleic acids. In some embodiments a polymerase has a relatively low dissociation rate for one or more natural and unnatural nucleic acids. In some embodiments a polymerase has a relatively high dissociation rate for one or more natural and unnatural nucleic acids. The dissociation rate is an activity of a polymerase that can be adjusted to tune reaction rates in methods set forth herein.

In some embodiments, polymerases are characterized according to their fidelity when used with a particular natural and/or unnatural nucleic acid or collections of natural and/or unnatural nucleic acid. Fidelity generally refers to the accuracy with which a polymerase incorporates correct nucleic acids into a growing nucleic acid chain when making a copy of a nucleic acid template. DNA polymerase fidelity can be measured as the ratio of correct to incorrect natural and unnatural nucleic acid incorporations when the natural and unnatural nucleic acid are present, e.g., at equal concentrations, to compete for strand synthesis at the same site in the polymerase-strand-template nucleic acid binary complex. DNA polymerase fidelity can be calculated as the ratio of (k_{cat}/Kₘ) for the natural and unnatural nucleic acid and (k_{cat}/Kₘ) for the incorrect natural and unnatural nucleic acid; where k_{cat} and Kₘ are Michaelis-Menten parameters in steady state enzyme kinetics (Fersht, A. R. (1985) Enzyme Structure and Mechanism, 2nd ed., p 350, W. H. Freeman & Co., New York., incorporated herein by reference). In some embodiments, a polymerase has a fidelity value of at least about 100, 1000, 10,000, 100,000, or 1×10⁶, with or without a proofreading activity.

In some embodiments, polymerases from native sources or variants thereof are screened using an assay that detects incorporation of an unnatural nucleic acid having a particular structure. In one example, polymerases can be screened for the ability to incorporate an unnatural nucleic acid or UBP; e.g., d5SICSTP, dCNMOTP, dTPT3TP, dNaMTP, dCNMOTP-dTPT3TP, or d5SICSTP-dNaMTP UBP. A polymerase, e.g., a heterologous polymerase, can be used that displays a modified property for the unnatural nucleic acid as compared to the wild-type polymerase. For example, the modified property can be, e.g., Kₘ, k_{cat}, Vₘₐₓ, polymerase processivity in the presence of an unnatural nucleic acid (or of a naturally occurring nucleotide), average template read-length by the polymerase in the presence of an unnatural nucleic acid, specificity of the polymerase for an unnatural nucleic acid, rate of binding of an unnatural nucleic acid, rate of product (pyrophosphate, triphosphate, etc.) release, branching rate, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the polymerase optionally has an increased rate of binding of an unnatural nucleic acid, an increased rate of product release, and/or a decreased branching rate, as compared to a wild-type polymerase.

At the same time, a polymerase can incorporate natural nucleic acids, e.g., A, C, G, and T, into a growing nucleic acid copy. For example, a polymerase optionally displays a specific activity for a natural nucleic acid that is at least about 5% as high (e.g., 5%, 10%, 25%, 50%, 75%, 100% or higher), as a corresponding wild-type polymerase and a processivity with natural nucleic acids in the presence of a template that is at least 5% as high (e.g., 5%, 10%, 25%, 50%, 75%, 100% or higher) as the wild-type polymerase in the presence of the natural nucleic acid. Optionally, the polymerase displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type polymerase.

Polymerases used herein that can have the ability to incorporate an unnatural nucleic acid of a particular structure can also be produced using a directed evolution approach. A nucleic acid synthesis assay can be used to screen for polymerase variants having specificity for any of a variety of unnatural nucleic acids. For example, polymerase variants can be screened for the ability to incorporate an unnatural nucleoside triphosphate opposite an unnatural nucleotide in a DNA template; e.g., dTPT3TP opposite dCNMO, dCNMOTP opposite dTPT3, NaMTP opposite dTPT3, or TAT1TP opposite dCNMO or dNaM. In some embodiments, such an assay is an in vitro assay, e.g., using a recombinant polymerase variant. In some embodiments, such an assay is an in vivo assay, e.g., expressing a polymerase variant in a cell. Such directed evolution techniques can be used to screen variants of any suitable polymerase for activity toward any of the unnatural nucleic acids set forth herein. In some instances, polymerases used herein have the ability to incorporate unnatural ribonucleotides into a nucleic acid, such as RNA. For example, NaM or TAT1 ribonucleotides are incorporated into nucleic acids using the polymerases described herein.

Modified polymerases of the compositions described can optionally be a modified and/or recombinant Φ29-type DNA polymerase. Optionally, the polymerase can be a modified and/or recombinant Φ29, B103, GA-1, PZA, Φ15, BS32, M2Y, Nf, G1, Cp-1, PRD1, PZE, SF5, Cp-5, Cp-7, PR4, PR5, PR722, or L17 polymerase.

Modified polymerases of the compositions described can optionally be modified and/or recombinant prokaryotic DNA polymerase, e.g., DNA polymerase II (Pol II), DNA polymerase III (Pol III), DNA polymerase IV (Pol IV), DNA polymerase V (Pol V). In some embodiments, the modified polymerases comprise polymerases that mediate DNA synthesis across non-instructional damaged nucleotides. In some embodiments, the genes encoding Pol I, Pol II (polB), Poll IV (dinB), and/or Pol V (umuCD) are constitutively expressed, or overexpressed, in the engineered cell, or SSO. In some embodiments, an increase in expression or overexpression of Pol II contributes to an increased retention of unnatural base pairs (UBPs) in an engineered cell, or SSO.

Nucleic acid polymerases generally useful in the present disclosure include DNA polymerases, RNA polymerases, reverse transcriptases, and mutant or altered forms thereof. DNA polymerases and their properties are described in detail in, among other places, DNA Replication 2^{nd} edition, Kornberg and Baker, W. H. Freeman, New York, N. Y. (1991). Known conventional DNA polymerases useful in the present disclosure include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (TIi) DNA polymerase (also referred to as Vent™ DNA polymerase, Cariello et al, 1991, Polynucleotides Res, 19: 4193, New England Biolabs), 9° Nm™ DNA polymerase (New England Biolabs), Stoffel fragment, Thermo Sequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al, 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al, 1981, J Biol. Chem. 256:3112), and archaeal DP1I/DP2 DNA polymerase II (Cann et al, 1998, Proc. Natl. Acad. Sci. USA 95:14250). Both mesophilic polymerases and thermophilic polymerases are contemplated. Thermophilic DNA polymerases include, but are not limited to, ThermoSequenase®, 9° Nm™, Therminator™, Taq, Tne, Tma, Pfu, Tfl, Tth, TIi, Stoffel fragment, Vent™ and Deep Vent™ DNA polymerase, KOD DNA polymerase, Tgo, JDF-3, and mutants, variants and derivatives thereof. A polymerase that is a 3′ exonuclease-deficient mutant is also contemplated. Reverse transcriptases useful in the present disclosure include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al, CRC Crit Rev Biochem. 3:289-347(1975)). Further examples of polymerases include, but are not limited to 9° N™ DNA Polymerase, Taq DNA polymerase, Phusion® DNA polymerase, Pfu DNA polymerase, RB69 DNA polymerase, KOD DNA polymerase, and VentR® DNA polymerase Gardner et al. (2004) “Comparative Kinetics of Nucleotide Analog Incorporation by Vent DNA Polymerase (J. Biol. Chem., 279(12), 11834-11842; Gardner and Jack “Determinants of nucleotide sugar recognition in an archaeon DNA polymerase” Nucleic Acids Research, 27(12) 2545-2553.) Polymerases isolated from non-thermophilic organisms can be heat inactivatable. Examples are DNA polymerases from phage. It will be understood that polymerases from any of a variety of sources can be modified to increase or decrease their tolerance to high temperature conditions. In some embodiments, a polymerase can be thermophilic. In some embodiments, a thermophilic polymerase can be heat inactivatable. Thermophilic polymerases are typically useful for high temperature conditions or in thermocycling conditions such as those employed for polymerase chain reaction (PCR) techniques.

In some embodiments, the polymerase comprises Φ29, B103, GA-1, PZA, Φ15, BS32, M2Y, Nf, GI, Cp-1, PRD1, PZE, SF5, Cp-5, Cp-7, PR4, PR5, PR722, L17, ThermoSequenase®, 9° Nm™, Therminator™ DNA polymerase, Tne, Tma, Tfl, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1I/DP2 DNA polymerase II, 9° N™ DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase I (or Klenow fragment), Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

### Nucleotide Transporter

Nucleotide transporters (NTs) are a group of membrane transport proteins that facilitate the transfer of nucleotide substrates across cell membranes and vesicles. In some embodiments, there are two types of NTs, concentrative nucleoside transporters and equilibrative nucleoside transporters. In some instances, NTs also encompass the organic anion transporters (OAT) and the organic cation transporters (OCT). In some instances, nucleotide transporter is a nucleoside triphosphate transporter (NTT).

In some embodiments, a nucleoside triphosphate transporter (NTT) is from bacteria, plant, or algae. In some embodiments, a nucleotide nucleoside triphosphate transporter is TpNTTT, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT 1, PamNTT2 (Protochlamydia amoebophila), CcNTT (Caedibacter caryophilus), or RpNTT1 (Rickettsia prowazekii). In some embodiments, the NTT is CNT1, CNT2, CNT3, ENT1, ENT2, OAT1, OAT3, or OCT1. In some instances, the NTT is PtNTT1, PtNTT2, PtNTT3, PtNTT4, PtNTT5, or PtNTT6.

In some embodiments, NTT imports unnatural nucleic acids into an organism, e.g. a cell. In some embodiments, NTTs can be modified such that the nucleotide binding site of the NTT is modified to reduce steric entry inhibition of the unnatural nucleic acid into the nucleotide biding site. In some embodiments, NTTs can be modified to provide increased interaction with one or more natural or unnatural features of the unnatural nucleic acids. Such NTTs can be expressed or engineered in cells for stably importing a UBP into the cells.

Accordingly, the present disclosure includes compositions that include a heterologous or recombinant NTT and methods of use thereof.

NTTs can be modified using methods pertaining to protein engineering. For example, molecular modeling can be carried out based on crystal structures to identify the locations of the NTTs where mutations can be made to modify a target activity or binding site. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of NTTs can be used in a methods or compositions set forth herein including, for example, protein-based enzymes isolated from biological systems and functional variants thereof. Reference to a particular NTT, such as those exemplified below, will be understood to include functional variants thereof unless indicated otherwise. In some embodiments, an NTT is a wild type NTT. In some embodiments, an NTT is a modified, or mutant, NTT.

In some embodiments, the modified or mutated NTTs as used herein is an NTT that is truncated at N-terminus, at C-terminus, or at both N and C-terminus. In some embodiments, the truncated NTT is at least 60%, at least 65%, at least 70%, at least 75%, at least 80%, at least 85%, or at least 90% identical the untruncated NTT. In some instances, the NTTs as used herein is PtNTT1, PtNTT2, PtNTT3, PtNTT4, PtNTT5, or PtNTT6. In some cases, the PtNTTs as used herein is truncated at N-terminus, at C-terminus, or at both N and C-terminus. In some embodiments, the truncated PtNTTs is at least 60%, at least 65%, at least 70%, at least 75%, at least 80%, at least 85%, or at least 90% identical the untruncated PtNTTs. In some cases, the NTT as used herein is a truncated PtNTT2, where the truncated PtNTT2 has an amino acid sequence that is at least 60%, at least 65%, at least 70%, at least 75%, at least 80%, at least 85%, or at least 90% identical to the amino acid sequence of untruncated PtNTT2. An example of untruncated PtNTT2 (NCBI accession number EEC49227.1, GI:217409295) has the amino acid sequence SEQ ID NO: 1.

NTTs, with features for improving entry of unnatural nucleic acids into cells and for coordinating with unnatural nucleotides in the nucleotide biding region, can also be used. In some embodiments, a modified NTT has a modified nucleotide binding site. In some embodiments, a modified or wild type NTT has a relaxed specificity for an unnatural nucleic acid. For example, an NTT optionally displays a specific importation activity for an unnatural nucleotide that is at least about 0.1% as high (e.g., about 0.1%, 0.2%, 0.5%, 0.8%, 1%, 1.1%, 1.2%, 1.5%, 1.8%, 2%, 3%, 4%, 5%, 10%, 25%, 50%, 75%, 100% or higher), as a corresponding wild-type NTT. Optionally, the NTT displays a k_{cat}/Kₘ or Vₘₐₓ/Kₘ for an unnatural nucleotide that is at least about 0.1% as high (e.g., about 0.1%, 0.2%, 0.5%, 0.8%, 1%, 1.1%, 1.2%, 1.5%, 1.8%, 2%, 3%, 4%, 5%, 10%, 25%, 50%, 75% or 100% or higher) as the wild-type NTT.

NTTs can be characterized according to their affinity for a triphosphate (i.e. Km) and/or the rate of import (i.e. Vmax). In some embodiments an NTT has a relatively Km or Vmax for one or more natural and unnatural triphosphates. In some embodiments an NTT has a relatively high Km or Vmax for one or more natural and unnatural triphosphates.

NTTs from native sources or variants thereof can be screened using an assay that detects the amount of triphosphate (either using mass spec, or radioactivity, if the triphosphate is suitably labeled). In one example, NTTs can be screened for the ability to import an unnatural triphosphate; e.g., dTPT3TP, dCNMOTP, d5SICSTP, dNaMTP, NaMTP, and/or TPT1TP. A NTT, e.g., a heterologous NTT, can be used that displays a modified property for the unnatural nucleic acid as compared to the wild-type NTT. For example, the modified property can be, e.g., Kₘ, k_{cat}, Vₘₐₓ, for triphosphate import. In one embodiment, the modified property is a reduced Kₘ for an unnatural triphosphate and/or an increased k_{cat}/Kₘ or Vₘₐₓ/Kₘ for an unnatural triphosphate. Similarly, the NTT optionally has an increased rate of binding of an unnatural triphosphate, an increased rate of intracellular release, and/or an increased cell importation rate, as compared to a wild-type NTT.

At the same time, an NTT can import natural triphosphates, e.g., dATP, dCTP, dGTP, dTTP, ATP, CTP, GTP, and/or TTP, into cell. In some instances, an NTT optionally displays a specific importation activity for a natural nucleic acid that is able to support replication and transcription. In some embodiments, an NTT optionally displays a k_{cat}/Kₘ or Vₘₐₓ/Kₘ for a natural nucleic acid that is able to support replication and transcription.

NTTs used herein that can have the ability to import an unnatural triphosphate of a particular structure can also be produced using a directed evolution approach. A nucleic acid synthesis assay can be used to screen for NTT variants having specificity for any of a variety of unnatural triphosphates. For example, NTT variants can be screened for the ability to import an unnatural triphosphate; e.g., d5SICSTP, dNaMTP, dCNMOTP, dTPT3TP, NaMTP, and/or TPT1TP. In some embodiments, such an assay is an in vitro assay, e.g., using a recombinant NTT variant. In some embodiments, such an assay is an in vivo assay, e.g., expressing an NTT variant in a cell. Such techniques can be used to screen variants of any suitable NTT for activity toward any of the unnatural triphosphate set forth herein.

### Nucleic Acid Reagents & Tools

A nucleotide and/or nucleic acid reagent (or polynucleotide) for use with methods, cells, or engineered microorganisms described herein comprise one or more ORFs with or without an unnatural nucleotide. An ORF may be from any suitable source, sometimes from genomic DNA, mRNA, reverse transcribed RNA or complementary DNA (cDNA) or a nucleic acid library comprising one or more of the foregoing and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleotide and/or nucleic acid reagent or other reagent described herein is isolated or purified. ORFs may be created that include unnatural nucleotides via published in vitro methods. In some cases, a nucleotide or nucleic acid reagent comprises an unnatural nucleobase.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media. In some instances, libraries of nucleic acid reagents are used with the methods and compositions described herein. For example, a library of at least 100, 1000, 2000, 5000, 10,000, or more than 50,000 unique polynucleotides are present in a library, wherein each polynucleotide comprises at least one unnatural nucleobase.

A nucleic acid or nucleic acid reagent, with or without an unnatural nucleotide, can comprise certain elements, e.g., regulatory elements, often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell-free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR. In some embodiments, the UTR can be optimized to alter or increase transcription or translation of the ORF that are either fully natural or that contain unnatural nucleotides.

Nucleic acid reagents, e.g., expression cassettes and/or expression vectors, can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleotide triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression and can be used to alter or optimize ORF expression, including ORFs that are fully natural or that contain unnatural nucleotides.

As noted above, nucleic acid reagents may also comprise one or more 5′ UTR's, and one or more 3′UTR's. For example, expression vectors used in eukaryotic host cells (e.g., yeast, fungi, insect, plant, animal, human or nucleated cells) and prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3″ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell-free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25-nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

Expression of a nucleotide triphosphate transporter from an expression cassette or expression vector can be controlled by any promoter capable of expression in prokaryotic cells or eukaryotic cells. A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with an RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions. As with all methods disclosed herein, the inclusion of natural or modified promoters can be used to alter or optimize expression of a fully natural ORF (e.g. an NTT or aaRS) or an ORF containing an unnatural nucleotide (e.g. an mRNA or a tRNA).

Non-limiting examples of selective or regulatory agents that influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; (13) nucleic acid segments that encode conditional replication functions, e.g., replication in certain hosts or host cell strains or under certain environmental conditions (e.g., temperature, nutritional conditions, and the like); and/or (14) nucleic acids that encode one or more mRNAs or tRNA that comprise unnatural nucleotides. In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

Nucleic acids encoding heterologous proteins, e.g., nucleotide triphosphate transporters, can be inserted into or employed with any suitable expression system. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids).

Recombinant expression is usefully accomplished using an expression cassette that can be part of a vector, such as a plasmid. A vector can include a promoter operably linked to nucleic acid encoding a nucleotide triphosphate transporter. A vector can also include other elements required for transcription and translation as described herein. An expression cassette, expression vector, and sequences in a cassette or vector can be heterologous to the cell to which the unnatural nucleotides are contacted. For example, a nucleotide triphosphate transporter sequence can be heterologous to the cell.

A variety of prokaryotic and eukaryotic expression vectors suitable for carrying, encoding and/or expressing nucleotide triphosphate transporters can be produced. Such expression vectors include, for example, pET, pET3d, pCR2.1, pBAD, pUC, and yeast vectors. The vectors can be used, for example, in a variety of in vivo and in vitro situations. Non-limiting examples of prokaryotic promoters that can be used include SP6, T7, T5, tac, bla, trp, gal, lac, or maltose promoters. Non-limiting examples of eukaryotic promoters that can be used include constitutive promoters, e.g., viral promoters such as CMV, SV40 and RSV promoters, as well as regulatable promoters, e.g., an inducible or repressible promoter such as a tet promoter, a hsp70 promoter, and a synthetic promoter regulated by CRE. Vectors for bacterial expression include pGEX-5X-3, and for eukaryotic expression include pCIneo-CMV. Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in Verma, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

### Cloning

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) modified or wild type nucleotide triphosphate transporters and/or polymerases), by engineering a microorganism with one or more ORFs of interest, which microorganism comprises altered activities of nucleotide triphosphate transporter activity or polymerase activity.

A nucleic acid may be specifically cleaved by contacting the nucleic acid with one or more specific cleavage agents. Specific cleavage agents often will cleave specifically according to a particular nucleotide sequence at a particular site. Examples of enzyme specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, BsaI, Bsm I, BsmBI, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., Sauer, Curr. Opin. Biotech. 5:521-527 (1994)). Other examples of recombination sites include attB, attP, attL, and attR sequences, and mutants, fragments, variants and derivatives thereof, which are recognized by the recombination protein k Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. Patent Appln. Nos. 09/517,466, and 09/732,914; U.S. Patent Publication No. US2002/0007051; and Landy, Curr. Opin. Biotech. 3:699-707 (1993)).

Examples of recombinase cloning nucleic acids are in Gateway® systems (Invitrogen, California), which include at least one recombination site for cloning desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A nucleic acid reagent sometimes contains one or more origin of replication (ORI) elements. In some embodiments, a template comprises two or more ORIs, where one functions efficiently in one organism (e.g., a bacterium) and another function efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent, e.g., an expression cassette or vector, can include nucleic acid sequence encoding a marker product. A marker product is used to determine if a gene has been delivered to the cell and once delivered is being expressed. Example marker genes include the E. coli lacZ gene which encodes β-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern et al., J. Molec. Appl. Genet. 1: 327 (1982)), mycophenolic acid, (Mulligan et al., Science 209: 1422 (1980)) or hygromycin, (Sugden, et al., Mol. Cell. Biol. 5: 410-413 (1985)).

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism, and other functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., P-lactamase), 0-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent can be of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (e.g., Biochemistry 1996 Jun. 25; 35(25):8429-38). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

Cloning strategies analogous to those described above may be employed to produce DNA containing unnatural nucleotides. For example, oligonucleotides containing the unnatural nucleotides at desired positions are synthesized using standard solid-phase synthesis and purified by HPLC. The oligonucleotides are then inserted into the plasmid containing required sequence context (i.e. UTRs and coding sequence) using a cloning method (such as Golden Gate Assembly) with cloning sites, such as BsaI sites (although others discussed above may be used).

### Kits and Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present disclosure for preparing genetically engineered cells. In one embodiment, a kit described herein includes a genetically engineered cell and one or more unnatural nucleic acids.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

Numbered Embodiments. The present disclosure includes the following non-limiting numbered embodiments:
Embodiment 1. A method of synthesizing an unnatural polypeptide comprising:
   a. providing at least one unnatural deoxyribonucleic acid (DNA) molecule comprising at least four unnatural base pairs;
   b. transcribing the at least one unnatural DNA molecule to afford a messenger ribonucleic acid (mRNA) molecule comprising at least two unnatural codons;
   c. transcribing the at least one unnatural DNA molecule to afford at least two transfer RNA (tRNA) molecules each comprising at least one unnatural anticodon, wherein the at least two unnatural base pairs in the corresponding DNA are in sequence contexts such that the unnatural codons of the mRNA molecule are complementary to the unnatural anticodon of each of the tRNA molecules; and d. synthesizing the unnatural polypeptide by translating the unnatural mRNA molecule utilizing the at least two unnatural tRNA molecules, wherein each unnatural anticodon directs site-specific incorporation of an unnatural amino acid into the unnatural polypeptide.
Embodiment 1.1. A method of synthesizing an unnatural polypeptide comprising:
   a. providing at least one unnatural deoxyribonucleic acid (DNA) molecule comprising at least four unnatural base pairs;
   b. transcribing the at least one unnatural DNA molecule to afford a messenger ribonucleic acid (mRNA) molecule comprising at least two unnatural codons;
   c. transcribing the at least one unnatural DNA molecule to afford at least two transfer RNA (tRNA) molecules each comprising at least one unnatural anticodon, wherein the at least two unnatural base pairs in the corresponding DNA are in sequence contexts such that one of the unnatural codons of the mRNA molecule is complementary to the unnatural anticodon of one of the tRNA molecules and at least one of the one or more other unnatural codons is complementary to the unnatural anticodon of at least one of the other the tRNA molecules; and d. synthesizing the unnatural polypeptide by translating the unnatural mRNA molecule utilizing the at least two unnatural tRNA molecules, wherein each unnatural anticodon directs site-specific incorporation of an unnatural amino acid into the unnatural polypeptide.
Embodiment 2. A method of synthesizing an unnatural polypeptide comprising:
   a. providing at least one unnatural deoxyribonucleic acid (DNA) molecule comprising at least four unnatural base pairs, wherein the at least one unnatural DNA molecule encodes (i) a messenger ribonucleic acid (mRNA) molecule comprising at least first and second unnatural codons and (ii) at least first and second transfer RNA (tRNA) molecules, the first tRNA molecule comprising a first unnatural anticodon and the second tRNA molecule comprising a second unnatural anticodon, and the at least four unnatural base pairs in the at least one DNA molecule are in sequence contexts such that the first and second unnatural codons of the mRNA molecule are complementary to the first and second unnatural anticodons, respectively;
   b. transcribing the at least one unnatural DNA molecule to afford the mRNA;
   c. transcribing the at least one unnatural DNA molecule to afford the at least first and second tRNA molecules; and
   d. synthesizing the unnatural polypeptide by translating the unnatural mRNA molecule utilizing the at least first and second unnatural tRNA molecules, wherein each of the at least first and second unnatural anticodons direct site-specific incorporation of an unnatural amino acid into the unnatural polypeptide.
Embodiment 3. The method of embodiment 1, 1.1., or 2, wherein the at least two unnatural codons each comprise a first unnatural nucleotide positioned at the first position, the second position, or the third position of the codon, optionally wherein the first unnatural nucleotide is positioned at the second position or the third position of the codon.
Embodiment 4. The method of any one of the preceding embodiments, wherein the at least two unnatural codons each comprises a nucleic acid sequence NNX, or NXN, and the unnatural anticodon comprises a nucleic acid sequence XNN, YNN, NXN, or NYN, to form the unnatural codon-anticodon pair comprising NNX-XNN, NNX-YNN, or NXN-NYN, wherein N is any natural nucleotide, X is a first unnatural nucleotide, and Y is a second unnatural nucleotide different from the first unnatural nucleotide, with X-Y or X-X forming the unnatural base pair in DNA.
Embodiment 4.1. The method of any one of the preceding embodiments, wherein the at least two unnatural codons each comprises a nucleic acid sequence XNN, NXN, NNX, and the unnatural anticodon comprises a nucleic acid sequence NNX, NNY, NXN, NYN, NNX, or NNY, to form the unnatural codon-anticodon pair comprising XNN-NNX, XNN-NNY, NXN-NXN, NXN-NYN, NNX-XNN, or NNX-YNN, wherein N is any natural nucleotide, X is a first unnatural nucleotide, and Y is a second unnatural nucleotide different from the first unnatural nucleotide, with X-X or X-Y forming the unnatural base pair in DNA.
Embodiment 5. The method of embodiment 4, wherein the codon comprises at least one G or C and the anticodon comprises at least one complementary C or G.
Embodiment 6. The method of embodiment 4 or 5, wherein X and Y are independently selected from the group consisting of
   (i) 2-thiouracil, 2′-deoxyuridine, 4-thio-uracil, uracil-5-yl, hypoxanthin-9-yl (I), 5-halouracil; 5-propynyl-uracil, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil;
   (ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1, 4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one);
   (iii) 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine;
   (iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and
   (v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.
Embodiment 7. The method of embodiment 4 or 5, wherein the bases comprising each of X and Y are independently selected from the group consisting of:

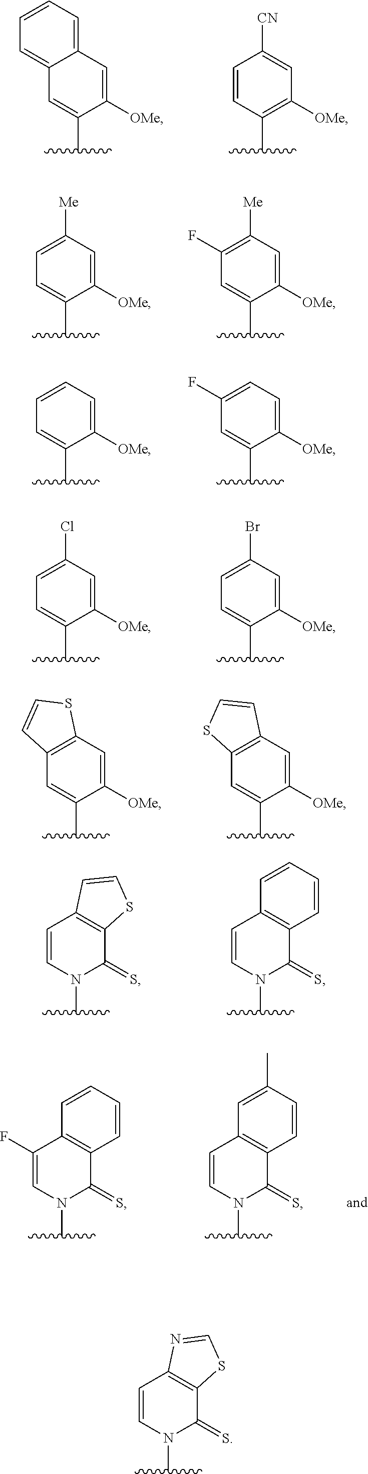

Embodiment 8. The method of embodiment 7, wherein the base comprising each X is OMe

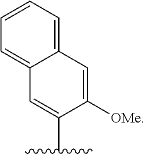 [SMILES: *c1cc2ccccc2cc1OC]

Embodiment 9. The method of embodiment 7 or 8, wherein the base comprising each Y is

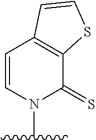 [SMILES: *n1ccc2ccsc2c1=S]

Embodiment 10. The method of any one of embodiments 4-9, wherein NNX-XNN is selected from the group consisting of UUX-XAA, UGX-XCA, CGX-XCG, AGX-XCU, GAX-XUC, CAX-XUG, AUX-XAU, CUX-XAG, GUX-XAC, UAX-XUA, and GGX-XCC.
Embodiment 11. The method of any one of embodiments 4-9, wherein NNX-YNN is selected from the group consisting of UUX-YAA, UGX-YCA, CGX-YCG, AGX-YCU, GAX-YUC, CAX-YUG, AUX-YAU, CUX-YAG, GUX-YAC, UAX-YUA, and GGX-YCC.
Embodiment 12. The method of any one of embodiments 4-9, wherein NXN-NYN is selected from the group consisting of GXU-AYC, CXU-AYG, GXG-CYC, AXG-CYU, GXC-GYC, AXC-GYU, GXA-UYC, CXC-GYG, and UXC-GYA.
Embodiment 13. The method of embodiment 12, wherein NXN-NYN is selected from the group consisting of AXG-CYU, GXC-GYC, AXC-GYU, GXA-UYC, CXC-GYG, and UXC-GYA.
Embodiment 13.1. The method of any one of embodiments 4.1-9, wherein XNN-NNY is selected from the group consisting of XUU-AAY, XUG-CAY, XCG-CGY, XAG-CUY, XGA-UCY, XCA-UGY, XAU-AUY, XCU-AGY, XGU-ACY, XUA-UAY, XUC-GAY, XCC-GGY, XAA-UUY, XAC-GUY, XGC-GCY, XGG-CCY, and XGG-CCY.
Embodiment 13.2. The method of any one of embodiments 4.1-9, wherein XNN-NNX is selected from the group consisting of XUU-AAX, XUG-CAX, XCG-CGX, XAG-CUX, XGA-UCX, XCA-UGX, XAU-AUX, XCU-AGX, XGU-ACX, XUA-UAX, XUC-GAX, XCC-GGX, XAA-UUX, XAC-GUX, XGC-GCX, XGG-CCX, and XGG-CCX.
Embodiment 14. The method of any one of the preceding embodiments, wherein the at least two unnatural tRNA molecules each comprises a different unnatural anticodon.
Embodiment 15. The method of embodiment 14, wherein the at least two unnatural tRNA molecules comprise a pyrrolysyl tRNA from the Methanosarcina genus and the tyrosyl tRNA from Methanocaldococcus jannaschii, or derivatives thereof.
Embodiment 16. The method of any one of embodiments 13, 14, or 15, comprising charging the at least two unnatural tRNA molecules by an amino-acyl tRNA synthetase.
Embodiment 17. The method of embodiment 16, wherein the amino acyl tRNA synthetase is selected from a group consisting of chimeric PylRS (chPylRS) and M. jannaschii AzFRS (MjpAzFRS).
Embodiment 18. The method of embodiment 14 or 15, comprising charging the at least two unnatural tRNA molecules by at least two tRNA synthetases.
Embodiment 19. The method of embodiment 18, wherein the at least two tRNA synthetases comprise chimeric PylRS (chPylRS) and M. jannaschii AzFRS (MjpAzFRS).
Embodiment 20. The method of any one of embodiments 1-19, wherein the unnatural polypeptide comprises two, three, or more unnatural amino acids.
Embodiment 21. The method of any one of embodiments 1-20, wherein the unnatural polypeptide comprises at least two unnatural amino acids that are the same.
Embodiment 22. The method of any one of embodiments 1-20, wherein the unnatural polypeptide comprises at least two different unnatural amino acids.
Embodiment 23. The method of any one of embodiments 1-22, wherein the unnatural amino acid comprises
   a lysine analogue;
   an aromatic side chain;
   an azido group;
   an alkyne group; or
   an aldehyde or ketone group.
Embodiment 24. The method of any one of the embodiments 1-22, wherein the unnatural amino acid does not comprise an aromatic side chain.
Embodiment 25. The method of any one of embodiments 1-22, wherein the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbornene lysine, TCO-lysine, methyltetrazine lysine, allyloxy carbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.
Embodiment 26. The method of any one of the preceding embodiments, wherein the at least one unnatural DNA molecule is in the form of a plasmid.
Embodiment 27. The method of any one of embodiments 1-26, wherein the at least one unnatural DNA molecule is integrated into the genome of a cell.
Embodiment 28. The method of embodiment 26 or 27, wherein the at least one unnatural DNA molecule encodes the unnatural polypeptide.
Embodiment 29. The method of any one of the preceding embodiments, wherein the method comprises the in vivo replication and transcription of the unnatural DNA molecule and the in vivo translation of the transcribed mRNA molecule in a cellular organism.
Embodiment 30. The method of embodiment 29, wherein the cellular organism is a microorganism.
Embodiment 31. The method of embodiment 30, wherein the cellular organism is a prokaryote.
Embodiment 32. The method of embodiment 31, wherein the cellular organism is a bacterium.
Embodiment 33. The method of embodiment 32, wherein the cellular organism is a gram-positive bacterium.
Embodiment 34. The method of embodiment 32, wherein the cellular organism is a gram-negative bacterium.
Embodiment 35. The method of embodiment 34, wherein the cellular organism is Escherichia coli.
Embodiment 36. The method of any one of the preceding embodiments, wherein the at least two unnatural base pairs comprise base pairs selected from dCNMO-dTPT3, dNaM-dTPT3, dCNMO-dTATT, or dNaM-dTATT.
Embodiment 37. The method of any one of embodiments 29-36, wherein the cellular organism comprises a nucleoside triphosphate transporter.
Embodiment 38. The method of embodiment 37, wherein the nucleoside triphosphate transporter comprises the amino acid sequence of PtNTT2.
Embodiment 39. The method of embodiment 38, wherein the nucleoside triphosphate transporter comprises a truncated amino acid sequence of PtNTT2.
Embodiment 40. The method of embodiment 39, wherein the truncated amino acid sequence of PtNTT2 is at least 80% identical to aPtNTT2 encoded by SEQ ID NO.1.
Embodiment 41. The method of any one of embodiments 29-40, wherein the cellular organism comprises the at least one unnatural DNA molecule.
Embodiment 42. The method of embodiment 41, wherein the at least one unnatural DNA molecule comprises at least one plasmid.
Embodiment 43. The method of embodiment 42, wherein the at least one unnatural DNA molecule is integrated into the genome of the cell.
Embodiment 44. The method of embodiment 42 or 43, wherein the at least one unnatural DNA molecule encodes the unnatural polypeptide.
Embodiment 45. The method of any one of embodiments 1-26, wherein the method is an in vitro method, comprising synthesizing the unnatural polypeptide with a cell-free system.
Embodiment 46. The method of any one of the preceding embodiments, wherein the unnatural base pairs comprise at least one unnatural nucleotide comprising an unnatural sugar moiety.
Embodiment 47. The method of embodiment 46, wherein the unnatural sugar moiety comprises a moiety selected from the group consisting of OH, substituted lower alkyl, alkaryl, aralkyl, β-alkaryl or β-aralkyl, SH, SCH₃, OCN, Cl, Br, CN, CF₃, OCF₃, SOCH₃, SO₂CH₃, ONO₂, NO₂, N3, NH₂F;
   O-alkyl, S-alkyl, N-alkyl;
   O-alkenyl, S-alkenyl, N-alkenyl;
   O-alkynyl, S-alkynyl, N-alkynyl;
   O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —O(CH₂)ₙON[(CH₂)ₙCH₃)]₂, wherein n and m are from 1 to about 10;
   and/or a modification at the 5′ position: 5′-vinyl, 5′-methyl (R or S);
   a modification at the 4′ position:
   4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.
Embodiment 48. A cell comprising at least one unnatural DNA molecule comprising at least four unnatural base pairs, wherein the at least one unnatural DNA molecule encodes (i) a messenger ribonucleic acid (mRNA) molecule encoding an unnatural polypeptide and comprising at least first and second unnatural codons and (ii) at least first and second transfer RNA (tRNA) molecules, the first tRNA molecule comprising a first unnatural anticodon and the second tRNA molecule comprising a second unnatural anticodon, and the at least four unnatural base pairs in the at least one DNA molecule are in sequence contexts such that the first and second unnatural codons of the mRNA molecule are complementary to the first and second unnatural anticodons, respectively.
Embodiment 49. The cell of embodiment 48, further comprising the mRNA molecule and the at least first and second tRNA molecules.
Embodiment 50. The cell of embodiment 49, wherein the at least first and second tRNA molecules are covalently linked to unnatural amino acids.
Embodiment 51. The cell of embodiment 50, further comprising the unnatural polypeptide.
Embodiment 52. A cell comprising:
   a. at least two different unnatural codon-anticodon pairs, wherein each unnatural codon-anticodon pair comprises an unnatural codon from unnatural messenger RNA (mRNA) and unnatural anticodon from an unnatural transfer ribonucleic acid (tRNA), said unnatural codon comprising a first unnatural nucleotide and said unnatural anticodon comprising a second unnatural nucleotide; and
   b. at least two different unnatural amino acids each covalently linked to a corresponding unnatural tRNA.
Embodiment 53. The cell of embodiment 52, further comprising at least one unnatural DNA molecule comprising at least four unnatural base pairs (UBPs).
Embodiment 54. The cell of any one of embodiments 48-53, wherein the first unnatural nucleotide is positioned at a second or a third position of the unnatural codon.
Embodiment 54.1. The cell of any one of embodiments 48-53, wherein the first unnatural nucleotide is positioned at a first, second, or a third position of the unnatural codon.
Embodiment 55. The cell of embodiment 54 or 54.1, wherein the first unnatural nucleotide is complementarily base paired with the second unnatural nucleotide of the unnatural anticodon.
Embodiment 56. The cell of any one of embodiments 48-55, wherein the first unnatural nucleotide and the second unnatural nucleotide comprise first and second bases, respectively, independently selected from the group consisting of

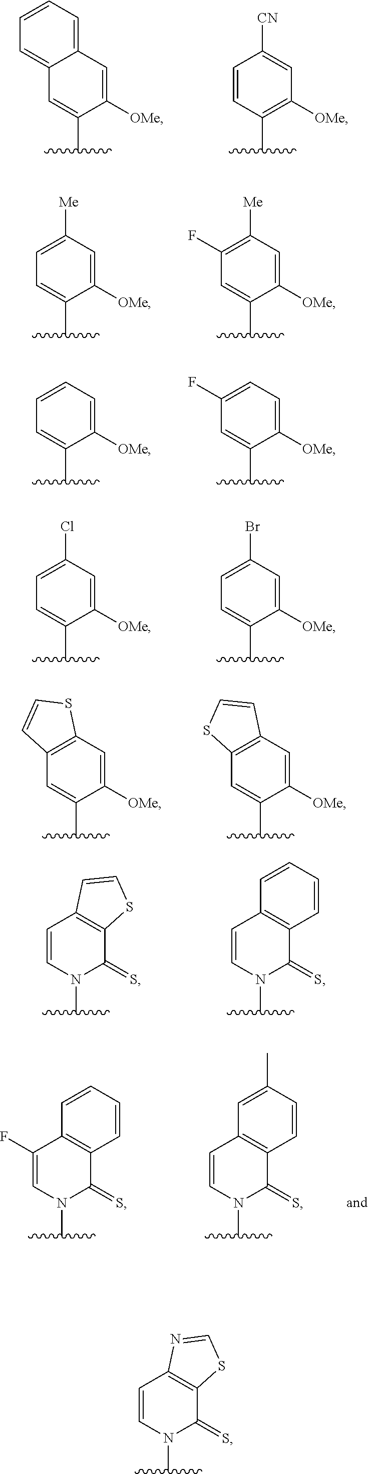

wherein the second base is different from the first base.
Embodiment 57. The cell of any one of embodiments 48 or 50-56, wherein the at least four unnatural base pairs are independently selected from the group consisting of dCNMO-dTPT3, dNaM-dTPT3, dCNMO-dTAT1, or dNaM-dTAT1.
Embodiment 58. The cell of any one of embodiments 48 or 50-57, wherein the at least one unnatural DNA molecule comprises at least one plasmid.
Embodiment 59. The cell of any one of embodiments 48 or 50-58, wherein the at least one unnatural DNA molecule is integrated into genome of the cell.
Embodiment 60. The cell of any one of embodiments 50-59, wherein the at least one unnatural DNA molecule encodes an unnatural polypeptide.
Embodiment 61. The cell of any one of embodiments 48-60, wherein the cell expresses a nucleoside triphosphate transporter.
Embodiment 62. The cell of embodiment 61 wherein the nucleoside triphosphate transporter comprises the amino acid sequence of PtNTT2.
Embodiment 63. The method of embodiment 62, wherein the nucleoside triphosphate transporter comprises a truncated amino acid sequence of PtNTT2.
Embodiment 64. The method of embodiment 63, wherein the truncated amino acid sequence of PtNTT2 is at least 80% identical to aPtNTT2 encoded by SEQ ID NO.1.
Embodiment 65. The cell of any one of embodiment 48 to 64, wherein the cell expresses at least two tRNA synthetases.
Embodiment 66. The cell of embodiment 65, wherein the at least two tRNA synthetases are chimeric PylRS (chPylRS) and M. jannaschii AzFRS (MjpAzFRS).
Embodiment 67. The cell of any one of embodiment 48 to 66, wherein the cell comprises unnatural nucleotides comprising an unnatural sugar moiety.
Embodiment 68. The cell of embodiment 67, wherein the unnatural sugar moiety is selected from the group consisting of:
   a modification at the 2′ position:
   OH, substituted lower alkyl, alkaryl, aralkyl, β-alkaryl or β-aralkyl, SH, SCH₃, OCN, Cl, Br, CN, CF₃, OCF₃, SOCH₃, SO₂CH₃, ONO₂, NO₂, N₃, NH₂F;
   O-alkyl, S-alkyl, N-alkyl;
   O-alkenyl, S-alkenyl, N-alkenyl;
   O-alkynyl, S-alkynyl, N-alkynyl;
   O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —O(CH₂)ₙON[(CH₂)ₙCH₃)]₂, wherein n and m are from 1 to about 10;
   and/or a modification at the 5′ position:
   5′-vinyl, 5′-methyl (R or S);
   a modification at the 4′ position:
   4′-S, heterocycloalkyl, heterocycloalkaryl, aminoalkylamino, poly alkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.
Embodiment 69. The cell of any one of embodiment 48 to 68, wherein at least one unnatural nucleotide base is recognized by an RNA polymerase during transcription.
Embodiment 70. The cell of any one of embodiment 48 to 69, wherein the cell translates at least one unnatural polypeptide comprising the at least two unnatural amino acids.
Embodiment 71. The cell of any one of embodiment 48 to 70, wherein the at least two unnatural amino acids are independently selected from the group consisting of N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.
Embodiment 72. The cell of any one of embodiments 48 to 71, wherein the cell is isolated.
Embodiment 73. The cell of any one of embodiments 48 to 72, wherein the cell is a prokaryote.
Embodiment 74. A cell line comprising the cell of any one of embodiments 48 to 73.

### EXAMPLES

### Example 1. Initial Codon Screen

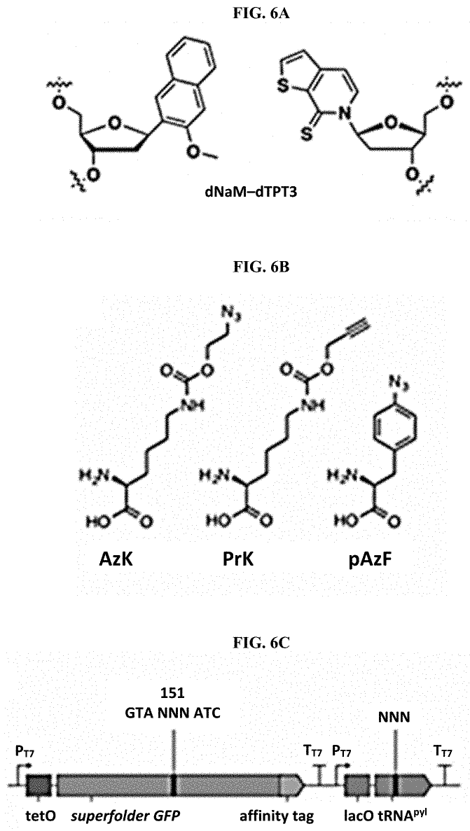

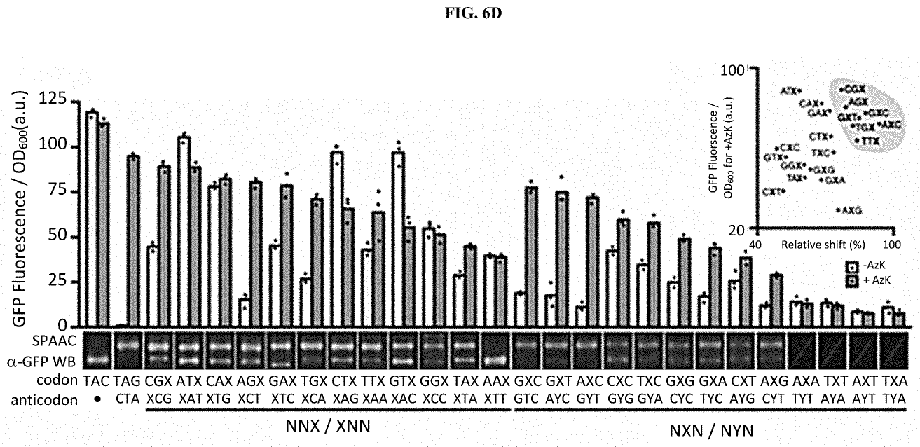

Green fluorescent protein and variants such as sfGFP have been used as model systems for the study of ncAA incorporation, especially at position Y151, which has been shown to tolerate a variety of natural and ncAA substitutions. Plasmids were constructed to contain two dNaM-dTPT3 UBPs, one positioned within codon 151 of sfGFP and the other positioned to encode the anticodon of M. mazei tRNA^{Pyl} (FIG. 6C), which was selectively charged by PylRS with the ncAA N6-((2-azidoethoxy)-carbonyl)-L-lysine (AzK) (FIG. 6B). Plasmids were constructed to examine the decoding of six codons, including two first position unnatural codons (XTC and XTG; X refers to dNaM), two second position unnatural codons (AXC and GXA), and two unnatural third position codons (AGX and CAX), as well as the opposite strand context codons (YTC, YTG, AYC, GYA, AGY, and CAY; Y refers to dTPT3).

While clonal populations of SSOs are able to produce larger quantities of pure unnatural protein, likely due to the elimination of plasmids that were misassembled during in vitro construction, to facilitate the initial codon screen protein expression was first explored with a non-clonal population of cells, and protein production was assayed immediately after transformation. Plasmids were used to transform E. coli ML2 (BL21(DE3) lacZYA:PtNTT2(66-575) ΔrecA polB++) that harbored an accessory plasmid encoding the chimeric pyrrolysyl-tRNA synthetase (chPylRS^{IPYE}) and after growth to early stationary phase in selective media supplemented with dNaMTP and dTPT3TP, cells were transferred to fresh media. Following growth to mid-exponential phase, the culture was supplemented with NaMTP, TPT3TP, and AzK, and isopropyl-o-D-thiogalactoside (IPTG) was added to induce expression of T7 RNA polymerase (T7 RNAP), chPylRS^{IPYE}, and tRNA^{Pyl}. After 1 h of additional growth, anhydrotetracycline (aTc) was added to induce expression of sfGFP, which was monitored by fluorescence.

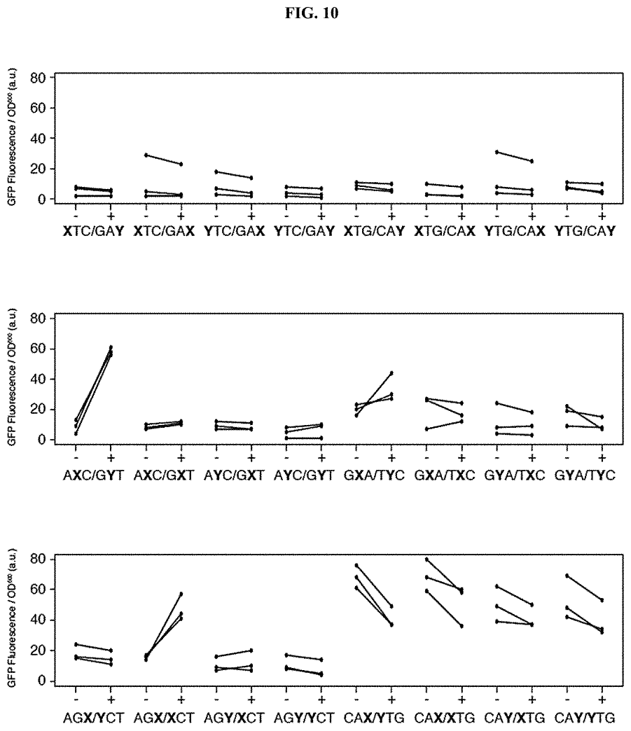

First position codons showed no significant fluorescence in the absence or presence of AzK, regardless of whether decoding was attempted with the heteropairing or self-pairing anticodons (e.g. tRNA^{Pyl}(CAY) or tRNA^{Pyl}(CAX), respectively, for XTG) (FIG. 10). Codons with dNaM at the second position showed little fluorescence in the absence of AzK, but in its presence showed significant fluorescence when decoded with tRNA^{Pyl} recoded with the heteropairing anticodons tRNA^{Pyl}(GYT) or tRNA^{Pyl}(TYC), but not with self-pairing anticodons tRNA^{Pyl}(GXT) or tRNA^{Pyl}(TXC). With dTPT3 at the second position, no fluorescence was observed with or without added AzK regardless of whether decoding was attempted with heteropairing or self-pairing tRNAs. The third position codons CAX and CAY showed high fluorescence in the absence of AzK, and surprisingly showed less with its addition, regardless of whether decoding was attempted with a heteropairing or self pairing tRNA^{Pyl}. This result suggests that the corresponding third position unnatural tRNAs nonproductively bind at the ribosome and block unnatural codon read-through by a natural tRNA. In the absence of AzK, AGX and AGY showed little fluorescence, and AGX with tRNA^{Pyl}(XCT) showed an increase in fluorescence with the addition of AzK.

As the first position codons did not appear promising, a more comprehensive screen of second position codons was conducted. Because the initial analysis indicated potential decoding only with NaM in the codon and with TPT3 in the anticodon, NXN codons and cognate tRNA^{Pyl}(NYN) were examined. Of the 16 possible codons, CXA, CXG, and TXG were excluded as the corresponding sequence context was poorly retained in the DNA of the SSO. In agreement with previous results, in the absence of AzK, the use of codons AXC and GXC resulted in little to no fluorescence, while in the presence of AzK, they resulted in significant fluorescence (FIG. 6D). Similarly, with the GXT, CXC, TXC, GXG, GXA, CXT, and AXG codons, the addition of AzK resulted in significant increases in fluorescence, relative to when AzK was withheld. The remaining four codons, AXA, AXT, TXA, and TXT, produced little fluorescence regardless of whether or not AzK was added, revealing a stringent requirement for at least one G-C pair.

To screen for unnatural protein production, sfGFP was purified via the C-terminal StrepII affinity tag and subjected to a strain-promoted azide-alkyne cycloaddition (SPAAC) reaction with dibenzocyclooctyne (DBCO) linked to a rhodamine dye (TAMRA) by four PEG units (DBCO-PEG₄-TAMRA). As shown previously, successful conjugation not only tags the proteins containing the ncAA with a detectable fluorophore, but also produces a detectable shift in electrophoretic mobility, allowing quantification of protein containing AzK relative to the total protein produced (i.e. fidelity of ncAA incorporation; FIG. 6D). In agreement with previous results, the use of codons GXC and AXC resulted in the production of significant amounts of sfGFP with the AzK residue. Remarkably, seven additional unnatural codons, GXT, CXC, TXC, GXG, GXA, CXT, and AXG, also yielded significant levels of unnatural protein (FIG. 6D, FIG. 11).

Finally, a more comprehensive screen of third position codons was conducted. Because in the initial screen only AGX appeared to be decoded, and only then by the self-pairing tRNA^{Pyl}(XCT), codons with dNaM at the third position of the codon with cognate self-pairing tRNA^{Pyl}(XNN) (FIG. 6C) were further examined. NCX codons were excluded as they result in sequence contexts of NCXA, which as noted above are not well retained in the DNA of the SSO. In agreement with the initial analysis, in the absence of AzK these codons generally resulted in more fluorescence than was observed with the second position codons, but in the presence of AzK variable increases in fluorescence were observed (FIG. 6D). Regardless, when protein was isolated and analyzed as described above, the use of CGX, ATX, CAX, AGX, GAX, TGX, CTX, TTX, GTX, or TAX all resulted in significant levels of unnatural protein production (FIG. 6D, FIG. 11). Codon GGX produced multiple shifted species, suggesting that tRNA^{Pyl}(XCC) decodes one or more natural codons. No unnatural protein was detected when codon AAX was used.

### Example 2. Codon Characterization in Clonal SSOs

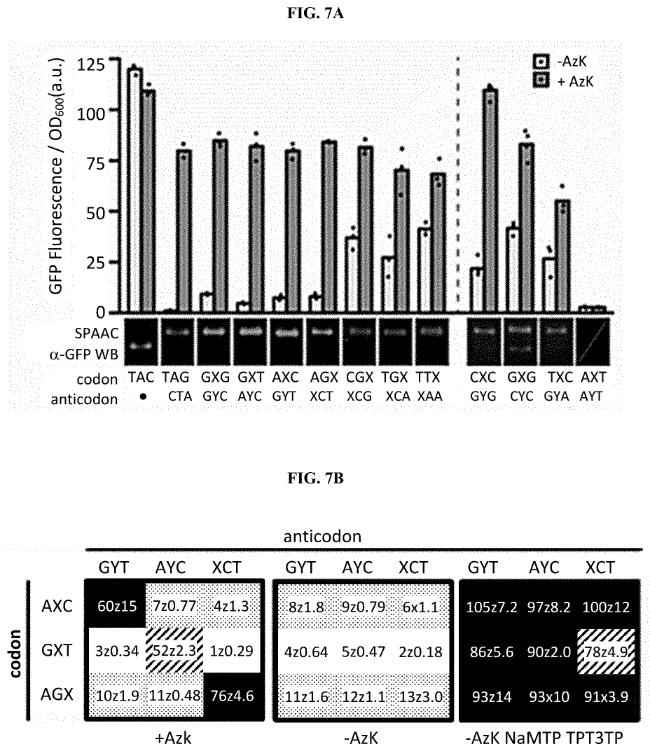

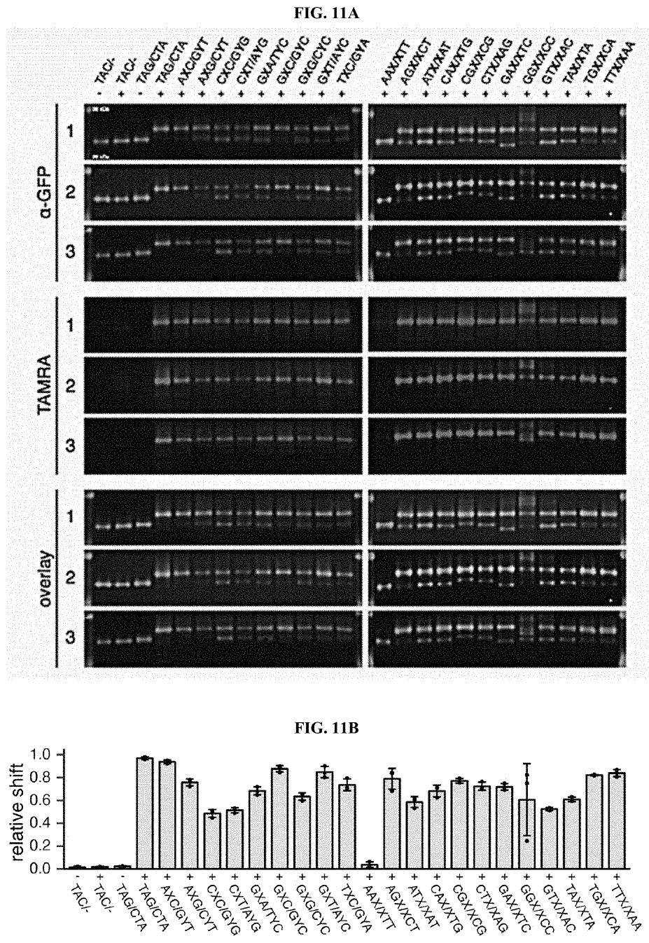

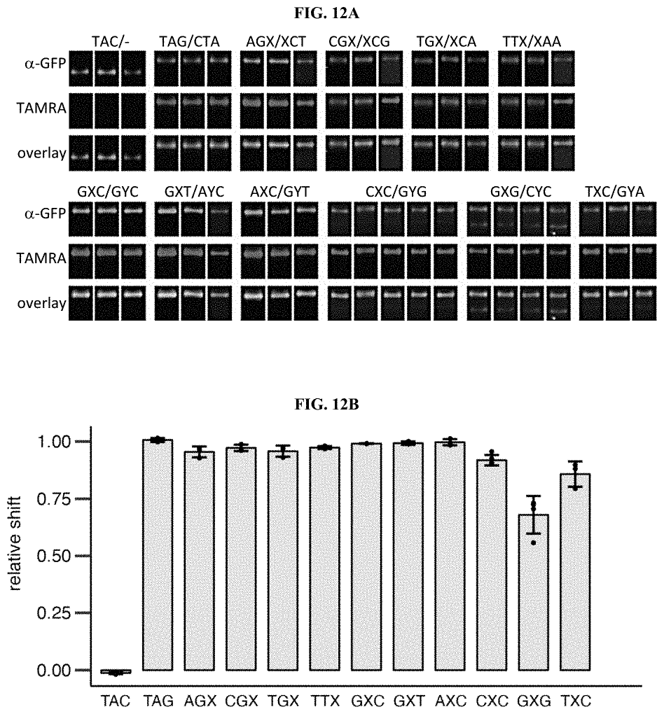

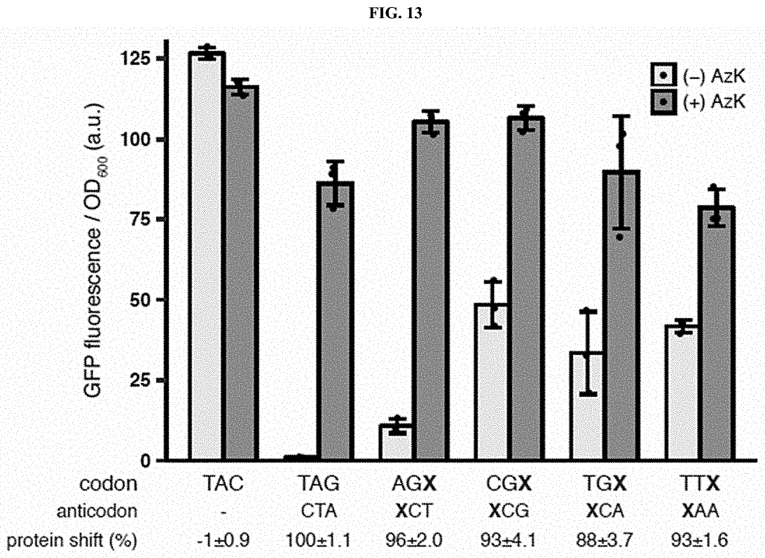

To select the most promising codon/anticodon pairs identified in the above described codon screen, the observed fluorescence in the presence of AzK and the induced mobility shift in isolated protein (FIG. 6D, inset) were compared. Based on this analysis, seven unnatural codon/anticodon pairs, GXC/GYC, GXT/AYC, AXC/GYT, AGX/XCT, CGX/XCG, TTX/XAA, TGX/XCA, were selected for further characterization. These codon/anticodon pairs were examined in clonal SSOs, which eliminates cells that were transformed with misassembled plasmids or plasmids that had lost the UBP during in vitro construction. Clonal SSOs were obtained by streaking transformants onto solid growth media containing dNaMTP and dTPT3TP, selecting individual colonies, and confirming plasmid integrity and high UBP retention. High retention clones were regrown and induced to produce protein as described above. Remarkably, the observed fluorescence indicates that each of the seven codon/anticodon pairs produces protein at a level that compares favorably with the amber suppression control, and moreover, the gel shift assay demonstrates that virtually all of the sfGFP contains the ncAA (FIG. 7A, FIG. 12). Decoding using codons/anticodons AGX/XCT, CGX/XCG, TTX/XAA, and TGX/XCA only depended on NaMTP in the expression media and produced sfGFP with a similar AzK content both with and without TPT3TP added (FIG. 13).

The seven unnatural codon/anticodon pairs analyzed above clearly mediated efficient decoding at the ribosome; however, it was possible that other codons from the preliminary non-clonal screen showed efficient decoding when analyzed in clonal SSOs. Thus, the unnatural protein production in clonal SSOs with four additional codon/anticodon pairs TXC/GYA, GXG/CYC, CXC/GYG, and AXT/AYT were explored. Despite high UBP retention (Table 1), AXT showed no fluorescence signal with or without AzK, further supporting the requirement for a G-C pair with the second position codons. Fluorescence with added AzK for TXC, CXC, and GXG was comparable to that of the seven initially characterized codons, although it was somewhat higher in the absence of AzK (FIG. 7A). SPAAC gel shift analysis revealed that CXC clearly resulted in significantly more shifted protein in the clonal SSO than observed in the preliminary screen with non-clonal SSOs, and TXC and GXG likely did as well, although the relatively larger error of the data from the preliminary screen precluded a quantitative comparison (FIG. 7B). The data suggested that for some codons, the suboptimal performance in the screen resulted, at least in part, from sequence-dependent differences in in vitro plasmid construction. Regardless, the results identified two additional high-fidelity codons, TXC and CXC, and suggested that more viable codons may yet be identified.

To begin to evaluate the orthogonality of unnatural codon/anticodon pairs, AXC/GYT, GXT/AYC, and AGX/XCT were selected and examined for protein production in clonal SSOs with all pairwise combinations of unnatural codons and anticodons. With added AzK, significant fluorescence was observed when each unnatural codon was paired with a cognate unnatural anticodon, and virtually no increase over background was observed when paired with a non-cognate unnatural anticodon (FIG. 7B). Thus, AXC/GYT, GXT/AYC, and AGX/XCT were orthogonal and capable of simultaneous use in the SSO.

### Example. 3 Simultaneous Decoding of Two Unnatural Codons

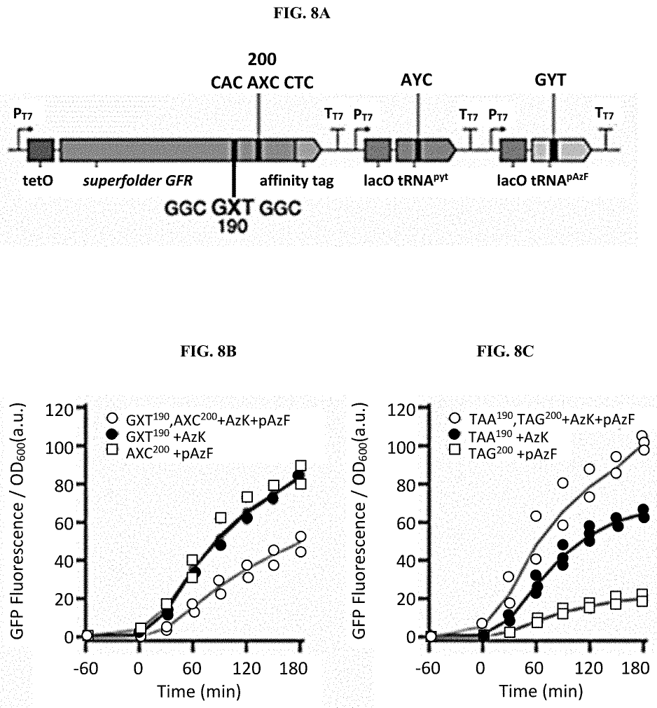

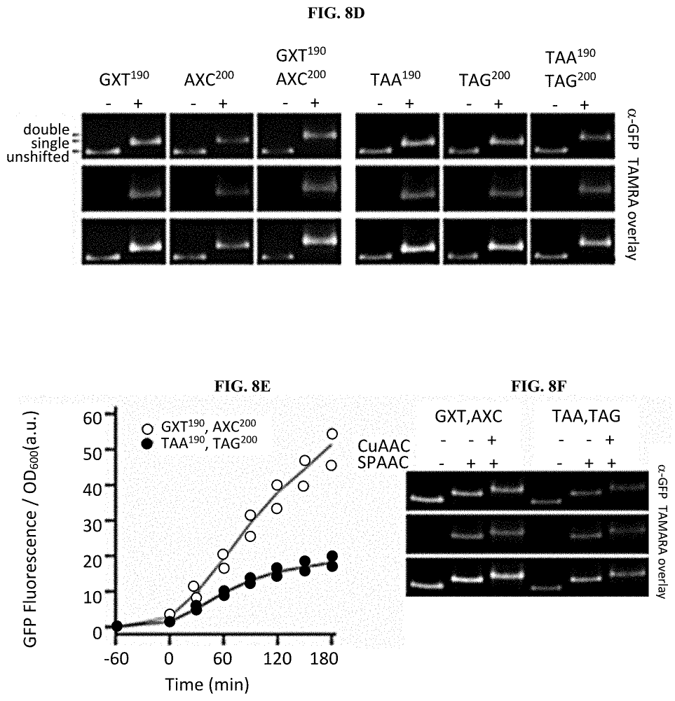

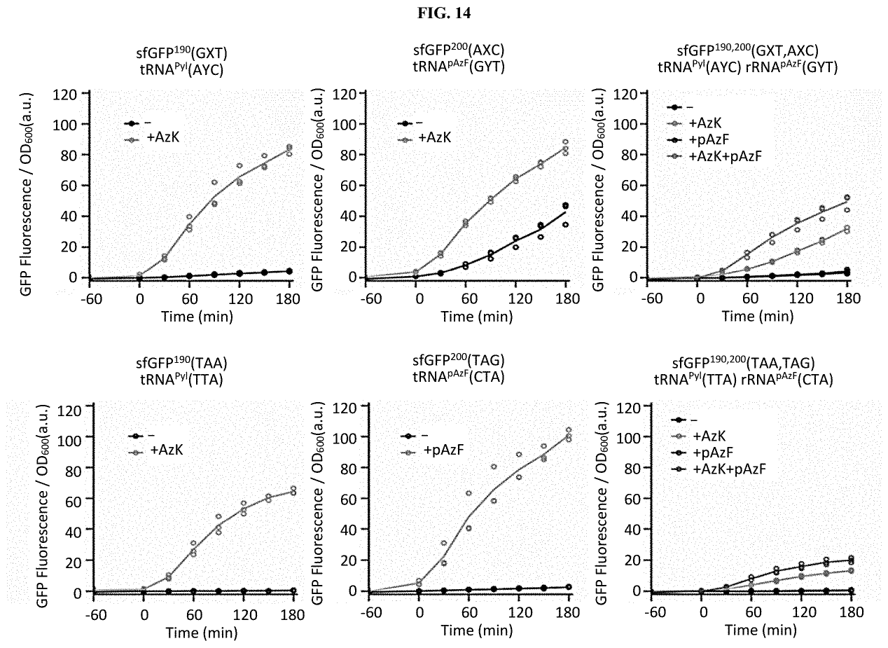

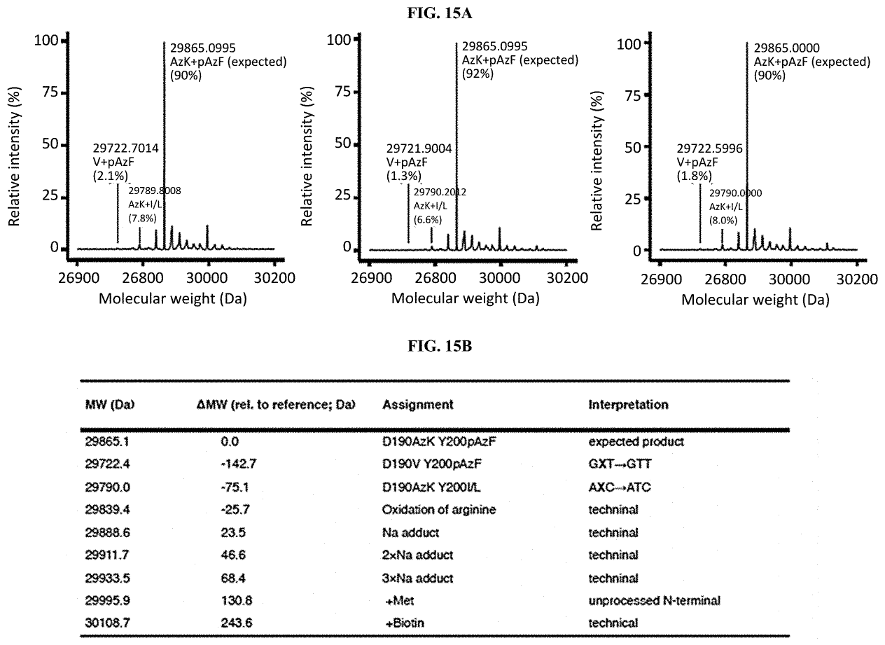

To explore the simultaneous decoding of multiple codons, a plasmid was first constructed with the native sfGFP codons at position 190 and 200 replaced by GXT and AXC, respectively (sfGFP^{190,200}(GXT,AXC)). In addition, the plasmid encoded both tRNA^{Pyl}(AYC) and M. jannaschii tRNA^{PAZ}F, which was selectively charged by M. jannaschii TyrRS (MfTyrRS) with p-azido-L-phenylalanine (pAzF; FIG. 6B), and whose anticodon was recoded to recognize AXC (tRNA^{pAzF}(GYT); FIG. 8A). E. coli ML2 harboring an accessory plasmid encoding both chPylRS^{IPYE} and MjpAzFRS, was transformed with the UBP-containing plasmid and clonal SSOs were obtained, grown, and induced to produce sfGFP as described above. With both AzK andpAzF provided, increased cell fluorescence was observed within the same timescale as expression with single codon constructs (FIG. 8B, FIG. 14) While the level of fluorescence with expression from sfGFP^{190,200}(GXT,AXC) was somewhat less than half that observed with sfGFP¹⁹⁰(GXT) or sfGFP²⁰⁰(AXC), it was significantly greater than that observed from an amber,ochre control (sfGFP^{190,200}(TAA,TAG)) decoded with the corresponding suppressor tRNAs (FIG. 8C, FIG. 14). In both cases, when analyzed by SPAAC gel shift, no unshifted band was apparent and the mobility of the major band was further retarded compared with that observed for the incorporation of a single ncAA, suggesting that indeed two ncAAs had been incorporated (FIG. 8D). To confirm that both pAzF and AzK were incorporated, purified protein was analyzed using quantitative intact protein mass spectrometry (HRMS ESI-TOF). In agreement with the gel shift assay, this analysis revealed that that 91±1.1% of the isolated protein contained bothpAzF and AzK, while 1.7±0.4% contained a singlepAzF and 7.5±0.78% a single AzK (FIG. 15). In both cases, the mass of the identified impurities correspond to the amino acid substitution consistent with a dX to dT mutation, suggesting that the majority of loss in ncAA incorporation fidelity resulted from loss of dNaM or dTPT3 during replication, and not due to errors during transcription or translation. Retention of UBPs based on the streptavidin-biotin shift assay. Retention comprised relative shift (i.e. signal of shifted band divided by total signal of shifted and unshifted bands) normalized to relative shift of ssDNA template control, except for tRNA^{pAxF} and tRNA^{Ser} where no normalization could be done. Mean standard deviation was shown (Table 1).

TABLE 1
Base pair (BP) retention in reported SSOs

| | | | | UBP retention | | UBP retention |
|---|---|---|---|---|---|---|
| | Appears | | Codon | codon | Anticodon | anticodon |
| Construct | in | n | (s) | (s) | (s) | (s) |
| Single codon | | | | | | |
| experiments | | | | | | |
| sfGFP¹⁵¹ M. | FIG. 6A | 3 | AXC | 94 ± 3 | GYT | 92 ± 4 |
| mazei tRNA^{Pyl} | | | | | | |
| sfGFP¹⁵¹ M. | FIG. 6A | 3 | GXC | 94 ± 3 | GYC | 96 ± 5 |
| mazei tRNA^{Pyl} | | | | | | |
| sfGFP¹⁵¹ M. | FIG. 6A | 3 | GXT | 99 ± 1 | AYC | 99 ± 1 |
| mazei tRNA^{Pyl} | | | | | | |
| sfGFP¹⁵¹ M. | FIG. 6A | 3 | AGX | 89 ± 3 | XCT | 61 ± 18 |
| mazei tRNA^{Pyl} | | | | | | |
| sfGFP¹⁵¹ M. | FIG. 6A | 3 | CGX | 89 ± 3 | XCG | 83 ± 8 |
| mazei tRNA^{Pyl} | | | | | | |
| sfGFP¹⁵¹ M. | FIG. 6A | 3 | TGX | 91 ± 2 | XCA | 78 ± 13 |
| mazei tRNA^{Pyl} | | | | | | |
| sfGFP¹⁵¹ M. | FIG. 6A | 3 | TTX | 95 ± 3 | XAA | 76 ± 37 |
| mazei tRNA^{Pyl} | | | | | | |
| sfGFP¹⁵¹ M. | FIG. 6A | 5 | CXC | 67 ± 8 | GYG | 91 ± 4 |
| mazei tRNA^{Pyl} | | | | | | |
| sfGFP¹⁵¹ M. | FIG. 6A | 4 | GXG | 58 ± 2 | CYC | 60 ± 10 |
| mazei tRN A^{Pyl} | | | | | | |
| sfGFP¹⁵¹ M. | FIG. 6A | 3 | TXC | 87 ± 6 | GYA | 94 ± 11 |
| mazei tRNA^{Pyl} | | | | | | |
| sfGFP¹⁵¹ M. | FIG. 6A | 3 | AXT | 97 ± 3 | AYT | 95 ± 1 |
| mazei tRNA^{Pyl} | | | | | | |
| sfGFP¹⁵¹ M. | FIG. 6B | 3 | AGX | 91 ± 1 | AYC | 101 ± l |
| mazei tRNA^{Pyl} | | | | | | |
| sfGFP¹⁵¹ M. | FIG. 6B | 3 | AGX | 92 ± 1 | GYT | 99 ± 6 |
| mazei tRNA^{Pyl} | | | | | | |
| sfGFP¹⁵¹ M. | FIG. 6B | 3 | AGX | 82 ± 3 | XCT | 100 ± 4 |
| mazei tRNA^{Pyl} | | | | | | |
| sfGFP¹⁵¹ M. | FIG. 6B | 3 | AXC | 96 ± 3 | AYC | 99 ± 2 |
| mazei tRNA^{Pyl} | | | | | | |
| sfGFP¹⁵¹ M. | FIG. 6B | 3 | AXC | 98 ± 1 | GYT | 94 ± 8 |
| mazei tRNA^{Pyl} | | | | | | |
| sfGFP¹⁵¹ M. | FIG. 6B | 3 | AXC | 99 ± 2 | XCT | 84 ± 12 |
| mazei tRNA^{Pyl} | | | | | | |
| sfGFP¹⁵¹ M. | FIG. 6B | 3 | GXT | 99 ± 4 | AYC | 97 ± 2 |
| mazei tRNA^{Pyl} | | | | | | |
| sfGFP¹⁵¹ M. | FIG. 6B | 3 | GXT | 100 ± 1 | GYT | 100 ± 1 |
| mazei tRNA^{Pyl} | | | | | | |
| sfGFP¹⁵¹ M. | FIG. 6B | 3 | GXT | 99 ± 1 | XCT | 101 ± 1 |
| mazei tRNA^{Pyl} | | | | | | |
| Multicodon codons | | | | | | |
| experiments | | | | | | |
| (including | | | | | | |
| controls) | | | | | | |
| sfGFP¹⁹⁰ M. | FIG. 7B | 3 | GXT | 103 ± 4 | AYC | 101 ± 4 |
| mazei tRNA^{Pyl} | | | | | | |
| sfGFP²⁰⁰ | FIG. 7B | 3 | AXC | 96 ± 2 | GYT | >94 ± 1 |
| M. jannaschii | | | | | | |
| sfGFP^{190, 200} | FIG. 7B | 3 | GXT, | 98 ± 3, | AYC, | 96 ± 1, >88 ± 1 |
| M. mazei | | | AXC | 86 ± 2 | GYT | |
| tRNA^{Pyl} | | | | | | |
| M. jannaschii | | | | | | |
| tRNA^{pAzF} | | | | | | |
| sfGFP^{151, 190, 200} | FIG. 7B | 3 | AXC, | 92 ± 1, | XCT, | 93 ± 3, >87 ± 3, >94 ± 2 |
| M. mazei | | | GXT, | 101 ± 2, | GYT, | |
| tRNA^{Pyl} | | | AGX | 96 ± 3 | AYC | |
| M. jannaschii | | | | | | |
| tRNA^{pAzF} | | | | | | |
| E. coli tRNA^{Ser} | | | | | | |

The SSO yielded 16±3.2 μg·ml⁻¹ of purified protein, whereas the amber, ochre suppression control yielded 6.8±1.1 μg·ml⁻¹ However, it was noted that the SSO culture grew to a lower density than the amber, ochre control cells, and when normalized for D600, the SSO yielded 13±1.6 μg·ml⁻¹ of purified protein, whereas amber, ochre suppression yielded 2.8±0.28 μg ml⁻¹, demonstrating that the SSO produced in excess of 4.5-fold more protein per OD600. All yields determined by sfGFP capture using excess Strep-Tactin XT beads during affinity purification. Yield normalized to final OD₆₀₀ at t=180 min of expression. Mean 2 standard deviation was shown (Table 2). Thus, the SSO efficiently produces unnatural protein with two ncAAs.

TABLE 2
Protein yield of sfGFP expressions

| | | | | Norm. |
|---|---|---|---|---|
| | | Codon(s)/ | Protein | Protein |
| | | antico- | yield | yield (μg/ |
| Construct | n | don(s) | (μg/ml) | ml/OD600) |
| sfGFP¹⁵¹ M. mazei | 3 | TAC/— | 66 ± 13 | 23 ± 1.9 |
| tRNA^{Pyl} | | | | |
| sfGFP¹⁵¹ M. mazei | 3 | TAG/CTA | 52 ± 11 | 18 ± 3.0 |
| tRNA^{Pyl} | | | | |
| sfGFP¹⁵¹ M. mazei | 3 | AXC/GYT | 28 ± 6.3 | 19 ± 2.1 |
| tRNA^{Pyl} | | | | |
| sfGFP¹⁵¹ M. mazei | 3 | GXC/GYC | 31 ± 0.32 | 18 ± 2.9 |
| tRNA^{Pyl} | | | | |
| sfGFP¹⁵¹ M. mazei | 3 | GXT/AYC | 29 ± 3.3 | 21 ± 0.22 |
| tRNA^{Pyl} | | | | |
| sfGFP¹⁵¹ M. mazei | 3 | AGX/XCT | 34 ± 4.7 | 19 ± 1.7 |
| tRNA^{Pyl} | | | | |
| sfGFP¹⁵¹ M. mazei | 3 | CGX/XCG | 29 ± 2.8 | 19 ± 5.2 |
| tRNA^{Pyl} | | | | |
| sfGFP¹⁵¹ M. mazei | 3 | TGX/XCA | 27 ± 3.2 | 18 ± 4.8 |
| tRNA^{Pyl} | | | | |
| sfGFP¹⁵¹ M. mazei | 3 | TTX/XAA | 27 ± 4.1 | 19 ± 4.6 |
| tRNA^{Pyl} | | | | |
| sfGFP^{190, 200} M. mazei | 3 | TAA, | 5.6 ± 1.0 | 5.0 ± 0.24 |
| tRNA^{Pyl}, M. jannaschii | | TAG/TTA, | | |
| tRNA^{pAzF} | | CTA | | |
| sfGFP^{190, 200} M. mazei | 3 | TAA, | 6.8 ± 1.1 | 2.8 ± 0.28 |
| tRNA^{Pyl}, M. jannaschii | | TAG/TTA, | | |
| tRNA^{pAzF} | | CTA | | |
| sfGFP^{190, 200} M. mazei | 3 | GXT, | 16 ± 3.2 | 13 ± 1.6 |
| tRNA^{Pyl}, M. jannaschii | | AXC/AYC, | | |
| tRNA^{pAzF} | | GYT | | |
| sfGFP^{151, 190, 200} M. | 3 | AXC, GXT, | 12 ± 1.9 | 7.8 ± 1.1 |
| mazei tRNA^{Pyl}, M. | | AGX/XCT, | | |
| jannaschii tRNA^{pAzF}, | | GYT, AYC | | |
| E. coli tRNA^{Ser} | | | | |

To characterize expression of proteins with ncAAs with different functional groups, sfGFP^{190,200}(GXT,AXC) was expressed in the SSO as described above but supplemented the growth medium with N⁶-(propargyloxy)-carbonyl-L-lysine (PrK, FIG. 61B), which was also recognized by chPylRS^{IPYE}, instead of AzK. No substantial impact on expression was observed by fluorescence for either the SSO or the amber, ochre control (FIG. 8E). In each case, it was verified that the correct incorporation of both PrK and pAzF by SPAAC with TAMRA-PEG₄-DBCO followed by copper-catalyzed alkyne-azide cycloaddition (CuAAC) using TAMRA-PEG₄-azide, as both induced an observable shift in electrophoretic mobility. Protein produced by the SSO, as well as the amber, ochre control, shows the expected gel shifts and TAMRA signal (FIG. 8F).

### Example 4. Simultaneous Decoding of Three Unnatural Codons

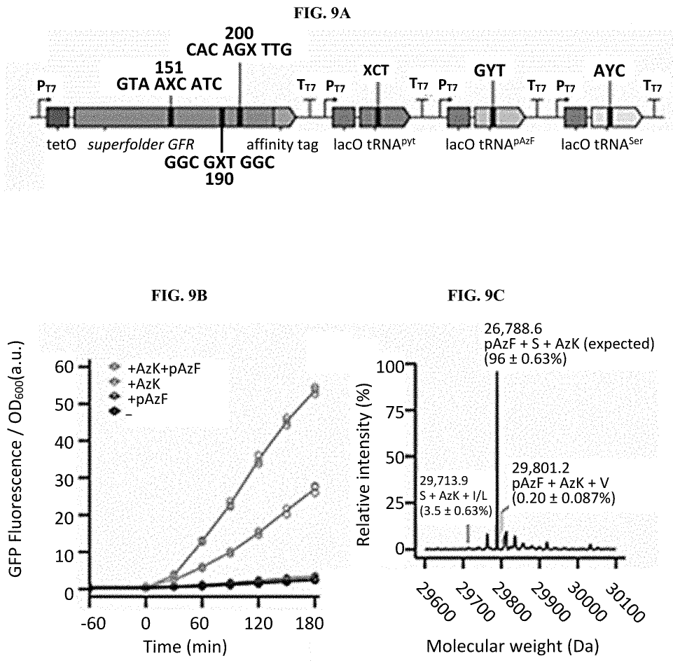

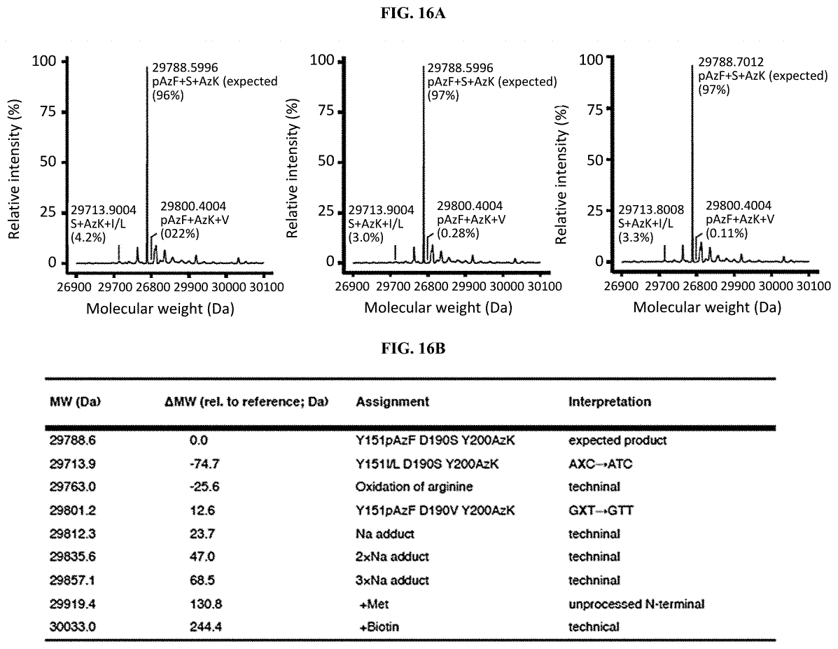

To explore the simultaneous decoding of the three orthogonal unnatural codons, the endogenous serine tRNA_{Ser}, E. coli SerT was employed, which was charged by endogenous SerRS without anticodon recognition and which was previously recoded to decode an unnatural codon. E. coli ML2 harboring an accessory plasmid encoding chPylRS^{IPYE} and MjpAzFRS was transformed with a plasmid expressing sfGFP^{151,190,200}(AXC,GXT,AGX) as well as tRNA^{Pyl}(XCT), tRNA^{pAzF}(GYT), and tRNA_{Ser}(AYC) (FIG. 9A), and clonal SSOs were prepared, grown, and induced to produce protein as described above. With AzK and pAzF added to the media, significant fluorescence was observed, similar to results obtained above for simultaneous decoding of two codons (FIG. 9B, FIG. 14). These cells yielded 12.1±1.9 μg ml⁻¹ (7.8±1.1 μg ml⁻¹ OD⁻¹), of isolated protein, which was only slightly less than the quantity isolated with the decoding of two unnatural codons (Table 2). To confirm that pAzF, AzK, and Ser had all been incorporated, purified protein was analyzed via quantitative intact protein mass spectrometry (HRMS ESI-TOF) and found that 96±0.63% of the isolated protein contained pAzF, AzK, and Ser, while the major impurity was sfGFP containing only AzK and Ser (3.5±0.63%). Protein without Ser incorporation was almost undetectable (0.20±0.087%), whereas a mass corresponding to protein containing only pAzF and Ser could not be detected (FIG. 9C, FIG. 16). Additionally, any impurities corresponding to the multiple insertion of either Ser, AzK, or pAzF were not detected.

### Example 5. Methods of In Vivo Expression of Unnatural Polypeptides

### Materials

A complete list of oligonucleotides and plasmids used is in Table 3. Natural ssDNA oligonucleotides and gBlocks were purchased from IDT (San Diego, Calif.). Genewiz (San Diego, Calif.) performed sequencing. All purification of DNA was carried out using Zymo Research silica column kits. All cloning enzymes and polymerases were purchased from New England Biolabs (Ipswich, Mass.). All bioconjugation reagents were purchased from Click Chemistry Tools (Scottsdale, Ariz.). All unnatural nucleoside triphosphates and nucleoside phosphoramidites used in this study were obtained from commercial sources. All ssDNA dNaM templates were also obtained from commercial sources, except sfGFP²⁰⁰(AGX) that was synthesized as described in the literature.

TABLE 3
Single-stranded DNA oligonucleotides used in PCR and
streptavidin-biotin shift assay

| | | | SEQ |
|---|---|---|---|
| | | | ID |
| ID | Application | Sequence (5' to 3') | NO: |
| Primers for | | | |
| UBP PCR | | | |
| Efo309 | sfGFP Y151 | ATGGGTCTCACACAAACTCGAGTACAACT | 2 |
| | insert F | TTAACTCACAC | |
| Efo310 | sfGFP Y151 | ATGGGTCTCGATTCCATTCTTTTGTTTGT | 3 |
| | insert R | CTGC | |
| Efo296 | sfGFP Y200 | CATAATGGTCTCGCTGCTGCCCGATAACC | 4 |
| | insert F | AC | |
| Efo297 | sfGFP Y200 | TGATATTGGTCTCGGTCTTTCGATAAAAC | 5 |
| | insert R | ACTCTGAGTAGAG | |
| Efo311 | M. mazei | ATGGGTCTCGAAACCTGATCATGTAGATC | 6 |
| | tRNA^{Py1} insert F | GAACGG | |
| Efo312 | M. mazei | ATGGGTCTCATCTAACCCGGCTGAACGG | 7 |
| | tRNA^{Py1} insert R | | |
| Efo313 | M. jannaschii | ATGGGTCTCCGGTAGTTCAGCAGGGCAGA | 8 |
| | tRNA^{pAzF} insert F | ACG | |
| Efo314 | M. jannaschii | ATGGGTCTCGGAGGGGATTTGAACCCCTG | 9 |
| | tRNA^{pAzF} insert R | CCATG | |
| Efo294 | sfGFP D190 | ATATTCGGTCTCGTCAGCAGAATACGCCG | 10 |
| | insert | ATTGG | |
| Efo295 | sfGFP D190 | ACGCGTTGGTCTCGGTTATCGGGCAGCAG | 11 |
| | insert | CACC | |
| YZ401 | E. coli tRNA^{Ser} | ATTGGTCTCGGCCGAGCGGTTGAAGGCAC | 12 |
| | insert F | | |
| YZ403 | E. coli tRNA^{Ser} | ATTGGTCTCTCTGGAACCCTTTCGGGTCG | 13 |
| | insert R | | |
| Primers for | | | |
| streptavid | | | |
| in-biotin | | | |
| shift assay | | | |
| Efo251 | Position Y151 | CTCGAGTACAACTTTAACTCACAC | 14 |
| | insert F | | |
| Efo252 | Position Y151 | GATTCCATTCTTTTGTTTGTCTGC | 15 |
| | insert R | | |
| Efo294 | Position D190 | ATATTCGGTCTCGTCAGCAGAATACGCCG | 10 |
| | insert F | ATTGG | |
| Efo295 | Position D190 | ACGCGTTGGTCTCGGTTATCGGGCAGCAG | 11 |
| | insert R | CACC | |
| Efo347 | Position Y200 | GCTGCTGCCCGATAACCAC | 16 |
| | insert F | | |
| Efo348 | Position Y200 | GGTCTTTCGATAAAACACTCTGAGTAGAG | 17 |
| | insert R | | |
| Efo343 | M. mazei | GAAACCTGATCATGTAGATCGAACGG | 18 |
| | tRNA^{Py1} insert F | | |
| Efo344 | M. mazei | ATCTAACCCGGCTGAACGG | 19 |
| | tRNA^{Py1} insert R | | |
| Efo313 | M. jannaschii | ATGGGTCTCCGGTAGTTCAGCAGGGCAGA | 8 |
| | tRNA^{pAzF} insert F | ACG | |
| Efo305 | M. jannaschii | CCGCTGCCACTAGGAAGCTTATG | 20 |
| | tRNA^{pAzF} insert R | | |
| Efo119 | E. coli tRNA^{Ser} | CCTCTAGAAAATCATTCCGGAAGTGTG | 21 |
| | insert F | | |
| Efol62 | E. coli tRNA^{Ser} | CTCTGGAACCCTTTCGGGTCGCCGGTTTG | 22 |
| | insert R | XTAGACCGGTGCCTTCAACCGCTCGGC | |
| Template | | | |
| for UBP | | | |
| PCR ([NNN] | | | |
| denotes any | | | |
| specified | | | |
| codon/ | | | |
| anticodon | | | |
| triplet) | | | |
| GFP151_[NNN] | sfGFP Y151 | CTCGAGTACAACTTTAACTCACACAATGT | 23 |
| | insert | A[NN]ATCACGGCAGACAAACAAAAGAA | |
| | | TGGAATC | |
| GFP190-GXT | sfGFP D190 | CAGCAGAATACGCCGATTGGCGXTGGCC | 24 |
| | insert | CGGTGCTGCTGCCCGATAACC | |
| GFP200_AXC | sfGFP Y200 | GCTGCTGCCCGATAACCACAXCCTCTCTA | 25 |
| | insert F | CTCAGAGTGTTTTATCGAAAGACC | |
| GFP200_opt_AGX | sfGFP Y200 | GCTGCCCGATAACCACAGXTTGTCTACTC | 26 |
| | insert R | AGAGTGTTTTATCG | |
| tRNA_Py1_[NNN] | M. mazei | GAATCTAACCCGGCTGAACGGATT[NNN] | 27 |
| | tRNA^{Py1} insert | AGTCCGTTCGATCTACATGATCAGG | |
| tRNA_Mj_GYT | M. mazei | GATTTGAACCCCTGCCATGCGGATTAXCA | 28 |
| | tRNA^{Py1} insert | GTCCGCCGTTCTGCCCTGCTGAA | |
| Trna_Eser_AYC | E. coli tRNA^{Ser} | CTCTGGAACCCTTTCGGGTCGCCGGTTTG | 22 |
| | insert | XTAGACCGGTGCCTTCAACCGCTCGGC | |

### Growth Conditions

All bacterial experiments were carried in 300 μl 2×YT (Fisher Scientific) media supplemented with potassium phosphate (50 mM pH 7). Growth was done in flat-bottomed 48-well plates (CELLSTAR, Greiner Bio-One) with shaking at 200 r.p.m. at 37° C. (Infors HT Minitron). Antibiotics were used at the following concentrations (unless otherwise noted): chloramphenicol (5 μg/ml), carbenicillin (100 μg/ml) and zeocin (50 μg/ml). Unnatural nucleoside triphosphates were used at the following concentrations (unless otherwise noted): dNaMTP (150 μM), dTPT3TP (10 μM), NaMTP (250 μM), TPT3TP (30 μM). UBP media is defined as said 2×YT media containing dNaMTP and dTPT3TP.

### Plasmid Construction

Large insertions (>100 bp), insertion of MjpAzFRS, tRNA or antibiotic resistance cassettes, were done by Gibson assembly of PCR amplicons or gBlocks. Amplicons were treated with DpnI over night at RT before assembly for 1.5 h at 50° C. Deletions or small insertions (<50 bp; e.g. codon or anticodon mutagenesis, removal of restriction sites, or introduction of golden gate destination sites) were constructed by introducing desired change into PCR primer overhangs designed to amplify the entire plasmid. Primers were phosphorylated using T4 PNK before PCR, and the resulting PCR amplicon was treated with DpnI over night at RT and recircularized using T4 DNA ligase. After initial assembly/ligation, plasmids were transformed into electrocompetent XL-10 Gold cells and grown on selective LB Lennox agar (BP Difco). Plasmids were isolated from individual colonies and were verified by Sanger sequencing before use. All plasmids used in this study can be found in Table 4. All sfGFP reading frames are controlled by P_{T7-tetO} and all tRNAs were controlled by P_{T7-lacO} Backbone pSYN contain: ori(p15A) bleoR. Backbone pGEX contain: ori(pBR322) ampR. Golden gate destination sites (dest) were composed of recognition sequences BsaI-KpnI-BsaI.

TABLE 4
Plasmids used in the Examples

| Backbone | Source | Application | Relevant properties |
|---|---|---|---|
| Superfolder GFP | | | |
| Expression | | | |
| plasmids | | | |
| pSYN | Zhang et al. ¹ | Natural | sfGFP¹⁵¹(TAG), M. mazei |
| | | expression | tRNA^{Pyl}(CCTA) |
| | | plasmid | |
| pSYN | Zhang et al. ¹ | Natural | sfGFP¹⁵¹(TAC) |
| | | expression | |
| | | plasmid | |
| pSYN | This work | Natural | sfGFP¹⁹⁰(TAA), M. mazei |
| | | expression | tRNA^{Pyl}(TTA), |
| | | plasmid | opal stop codon |
| pSYN | This work | Natural | sfGFP²⁰⁰(TAG), M. jannaschii |
| | | expression | tRNA^{pAzF}(CTA) |
| | | plasmid | |
| pSYN | This work | Natural | sfGFP^{190, 200}(TAA, TAG), M. mazei |
| | | expression | tRNA^{Pyl}(TTA) M. jannaschii |
| | | plasmid | tRNA^{pAzF}(CTA); opal stop codon |
| pSYN | Zhang et al. ¹ | UBP | sfGFP¹⁵¹(dest), M. mazei |
| | | destination | tRNA^{Pyl}(dest) |
| | | plasmid | |
| pSYN | This work | UBP | sfGFP¹⁹⁰(dest), M. mazei |
| | | destination | tRNA^{Pyl}(dest) |
| | | plasmid | |
| pSYN | This work | UBP | sfGFP²⁰⁰(dest), M. jannaschii |
| | | destination | tRNA^{pAzF}(dest) |
| | | plasmid | |
| pSYN | This work | UBP | sfGFP¹⁹⁰⁻²⁰⁰(dest), M. mazei tRNA^{Pyl}(dest), |
| | | destination | M. jannaschii tRNA^{pAzF}(dest) |
| | | plasmid | |
| pSYN | This work | UBP | sfGFP^{151, 190-200}(dest, dest), M. mazei |
| | | destination | tRNA^{Pyl}(dest), M. jannaschii |
| | | plasmid | tRNA^{pAzF}(dest), E. coli tRNA^{Ser}(dest) |
| Accessory | | | |
| plasmids | | | |
| pGEX | This work | Accessory | P_{AmpR}-tetR, P_{lacIq}-lacI, P_{tac-lacO}-chPylRS^{IPYE} |
| | | plasmid | |
| pGEX | This work | Accessory | P_{AmpR}-tetR, P_{lacIq}-lacI, P_{lacUV5}-lacO- |
| | | plasmid | MjpAzFRS, P_{tac-lacO}-chPylRS^{IPYE} |

¹ Zhang, Y. et al. A semi-synthetic organism that stores and retrieves increased genetic information. Nature 551, 644-647 (2017)

### PCR of UBP Oligos

Double-stranded DNA inserts with the UBP-containing sequence were obtained from PCR (OneTaq Standard Buffer 1×, 0.025 units/μl OneTaq, 0.2 mM dNTPs, 0.1 mM dTPT3TP, 0.1 mM dNaMTP, 1.2 mM MgSO₄, 1×SYBR Green, 1.0 μM primers, ˜20 pM template; cycling: 96° C. 0:30 min, 96° C. 0:30 min, 54° C. 0:30 min, 68° C. 4:00 min, fluorescence read, go to step 2<24 times) with primers (in list A) using chemically synthesized dNaM containing ssDNA oligonucleotides (in list B) as template. Inserts for position sfGFP¹⁹⁰ and sfGFP²⁰⁰ were combined by overlap extension using identical condition as above but with both templates at 1 nM. Amplifications were monitored and reactions were put on ice as the SYBR green trace plateaued. Products were analyzed via native PAGE (6% acrylamide:bisacrylamide 29:1; SYBR Gold stain in 1×TBE) to verify single amplicons, purified on a spin-column (Zymo Research), and quantified using Qubit dsDNA HS (ThermoFisher).

### Golden Gate Assembly of SSO Expression Vectors

UBP-containing inserts were incorporated into the pSYN entry vector framework (Table 4) via Golden Gate assembly (Cutsmart buffer 1×, 1 mM ATP, 6.67 units/μl T4 DNA ligase, 0.67 units/μl BsaI-HFv2, 20 ng/μl entry vector DNA; cycling: 37° C. 10:00 min, 37° C. 5:00 min, 16° C. 5:00 min, 22° C. 2:00 min, repeat from step 2 39 times, 37° C. 20:00 min, 55° C. 15:00 min, 80° C. 30:00 min) with 3:1 molar ratio of each insert to entry vector. BsaI-HF was used for experiments in FIG. 6. Residual linear DNA and undigested entry vector was digested with first KpnI-HF (0.33 units/μl, 1 h at 37° C.) followed by T5 exonuclease (0.17 units/μl, 30 min at 37° C.). Product was purified on a spin-column and quantified using Qubit dsDNA HS (ThermoFisher).

### Preparation of Competent Starter Cells

Strain ML2 (BL21(DE3) lacZYA::PtNTT2(66-575) ArecA polB⁺⁺) was transformed with the accessory pGEX plasmid (Table 4) and plated on LB Lennox agar with chloramphenicol and carbenicillin. Single colonies were picked and verified for PtNTT2 activity by uptake of radioactive [α-³²P]dATP as previously described (Zhang et al. 2017). Competent cells for UBP replication and translation were prepared by growth in 2×YT media at 37° C. 250 r.p.m. in a baffled culture flask until OD₆₀₀ 0.25-0.30. The cultures were transferred to pre-chilled 50 mL Falcon tubes and gently shaken in an ice-water bath for 2 min. Cells were pelleted by centrifugation (10 min, 3200 r.p.m) and washed in cold sterile water, pelleted and washed again, before finally being pelleted and suspended in 50 μl 10% glycerol per 10 mL culture. The cells were either used immediately or frozen at −80° C. for later use.

### Non-Clonal Population Experiments

Freshly prepared competent cells were electroporated (2.5 kV) with ˜0.4 ng Golden Gate assembly product and immediately suspended in 950 μl 2×YT supplemented with potassium phosphate (50 mM pH 7), whereof 10 μl was diluted into 40 μl of UBP media containing 1.25×dNaMTP and dTPT3TP without zeocin. After recovering the cells for 1 h at 37° C., 15 μl cells were suspended in 285 μl UBP media with zeocin and grown at 37° C. shaking in a 48-well plate. Cultures were transferred to ice before reaching stationary phase, at OD₆₀₀ ˜1, and stored overnight for protein expression.

### Clonal SSO Experiments

Competent cells were electroporated with Golden Gate assembly product (1-20 ng) and recovered as for non-clonal population experiments. Plating was carried out by spreading 10 μl recovery culture (and dilutions thereof) onto an agar droplets (250 μl 2×YT 2% agar 50 mM potassium phosphate) containing chloramphenicol, carbenicillin, zeocin, dNaMTP, and dTPT3TP. Colonies with approximately 0.5 mm in diameter were picked and suspended into UBP media (300 μl) after growth on the plate (12-20 h; 37° C.). Each culture was transferred to pre-chilled tubes on ice before reaching stationary phase, at OD˜1, and stored over night for protein expression. Each culture was prescreened for 1) UBP retention using the streptavidin biotin shift assay (as described below) and 2) qualitative sfGFP expression by mixing the culture 1:4 with media already containing the components for expression (ribonucleoside triphosphates, ncAAs, IPTG, and anhydrotetracycline). Colonies were discarded if they did not produce any fluorescent signal when the appropriate ncAA was added after 2 h of incubation at 37° C. or overnight at RT. Additionally, colonies with <80% UBP retention in sfGFP were discarded. If more than three colonies satisfied these criteria, then only the three with highest UBP retention were chosen to limit material expenses. The data to the right of the dashed line in FIG. 7A were obtained through slightly modified methods. Instead of prescreening colonies as described above, expression was carried out on numerous colonies, but protein analysis was only performed for cultures that showed promising fluorescence during expression. During expression 10 mM AzK was used. Additionally, buffer W2 was used during protein purification instead of buffer W.

### Precloned SSO Expression Vectors

In the experiments in FIG. 7B, FIG. 8, and FIG. 9 plasmids from prescreened colonies were isolated (Zymo Research Miniprep) to serve as starting plasmid for (precloned) transformation in order to ease colony prescreening. Plasmids were prescreened (as described above) for qualitative fluorescence from sfGFP expression with the appropriate ncAA(s). Colonies for the data in FIG. 7B were instead prescreened with and without rNaMTP and rTPT3TP in the presence of AzK to qualitatively produce a dark and a fluorescent signal, respectively. All precloned plasmids were prescreened for UBP retention in sfGFP (>80%). Furthermore, these plasmids were PCR amplified using a standard OneTaq protocol (New England Biolabs), without unnatural nucleoside triphosphates to force dX to dN mutations, and the amplicon was Sanger sequenced to verify integrity of the natural sequence in the plasmid. Silent mutations were allowed in protein coding sequences.

### UBP Protein Expression

Cultures were refreshed in UBP media to OD₆₀₀ 0.10-0.15 and 37° C. shaking until OD 0.5-0.8 when ribonucleotide triphosphates were added to 250 μM NaMTP and 30 μM TPT3TP, alongside ncAAs at 5 mMpAzF, 20 mM AzK, or 10 mM PrK. Only 10 mM AzK was used in double/triple codon experiments or controls thereof (FIG. 8, FIG. 9). After 20 min of further incubation, preinduction was initiated by adding IPTG (1 mM) and the cultures were incubated for 1 h further. Finally, sfGFP expression was induced by derepression of tetO by adding anhydrotetracycline (100 ng/μl). OD₆₀₀ and GFP fluorescence was monitored (every 30 min) using Perkin Elmer Envision 2103 Multilabel Reader (OD: 590/20 nm filter; sfGFP: ex. 485/14 nm, em. 535/25 nm). After 3 h of expression, cultures were pelleted and stored at −80° C. for later analysis.

### Streptavidin-Biotin Shift Assay for UBP Retention

UBP retention in plasmid DNA was determined by PCR amplification using unnatural nucleoside triphosphate d5SICSTP as well as the biotinylated dNaM analog dMMO2^{Bio}TP. Plasmids from SSOs were isolated via standard miniprep, resulting in a mixture of SSO expression plasmids (pSYN) and accessory plasmids (pGEX). A total of 2 ng of the plasmid mixture was used as a template in a 15 μl PCR reaction (OneTaq Standard Buffer 1×, 0.018 units/μl OneTaq, 0.007 units/μl DeepVent, 0.4 mM dNTPs, 0.1 mM d5SICSTP, 0.1 mM dMMO2^{Bio}TP, 2.2 mM MgSO₄, 1×SYBR Green, 1.0 μM primers; cycling: 96° C. 2:00 min, 96° C. 0:30 min, 50° C. 0:10 min, 68° C. 4:00 min, fluorescence read, 68° C. 0:10 min, go to step 2 <24 times). Individual samples were removed during the last step of each cycle as the SYBR Green I trace showed amplification to plateau. The resulting biotinylated amplicon was supplemented with 10 μg streptavidin (Promega) per 1.5-2.0 μl crude PCR reaction. The streptavidin bound fraction was visualized as a shift by 6% native-PAGE and both shifted and unshifted bands were quantified by ImageStudioLite or Fiji to yield the relative raw percentage of shift. By normalizing the raw shift to a control shift, generated by templating the PCR reaction with the chemically synthesized oligonucleotide, the overall UBP retention was assessed. Normalization was not possible for tRNA^{pAZF} or tRNA^{Ser} as faithful amplification was only possible with primers annealing outside the Golden Gate insert and thus did not anneal to the corresponding control oligonucleotide.

### Protein Purification

Cell pellets from protein expression experiments (200 μl) were lyzed using BugBuster (100 μl; EMD Millipore; 15 min; RT; 220 r.p.m.). Cell lysates were then diluted in Buffer W (50 mM HEPES pH 8, 150 mM NaCl, 1 mM EDTA) to a final volume equal to 500 μl minus the volume of affinity beads used. Magnetic Strep-Tactin XT beads (5% (v/v) suspension of MagStrep “type3” XT beads, IBA Lifesciences) were used at 20 μl for routine purification and 100 μl for estimation of total expression yield. Protein was bound to beads (30 min; 4° C.; gently rotation) before beads were pulled down and washed with Buffer W (2×500 μl). In protein purification for HRMS analysis Buffer W2 was used (50 mM HEPES pH 8, 1 mM EDTA) instead. Finally, protein was eluted using 25 μl Buffer BXT (50 mM HEPES pH 8, 150 mM NaCl, 1 mM EDTA, 50 mM d-Biotin) for 10 min at RT with occasional vortexing. Protein was eluted with buffer BXT2 (50 mM HEPES pH 8, 1 mM EDTA, 50 mM d-Biotin) for HRMS analysis. Qubit Protein Assay Kit (ThermoFisher) was used for quantification.

Western Blotting of TAMRA Conjugated sfGFP

SPAAC was carried out by incubation of 33 ng/μl pure protein with 0.1 mM TAMRA-PEG₄-DBCO (Click Chemistry Tools) over night at RT in darkness. The reactions were mixed 2:1 with SDS-PAGE loading dye (250 mM Tris-HCl pH 6, 30% glycerol, 5% OME, 0.02% bromophenol blue) and denatured for 5 min at 95° C. SDS-PAGE gel were 5% acrylamide stacking gels and 15% acrylamide resolution gel when analyzing position sfGFP¹⁵¹ and 17% for when analyzing sfGFP^{190,200} (resolution gel: 15% or 17% acrylamide:bisacrylamide 29:1, 0.1% (w/v) APS, 0.04% TEMED, 0.375 M Tris-HCl pH 8.8, 0.1% (w/v) SDS; stacking: 5% acrylamide:bisacrylamide 29:1, 0.1% (w/v) APS, 0.1% TEMED, 0.125 M Tris-HCl pH 6.8, 0.1% (w/v) SDS). Electrophoresis was carried out for 15 min at 40 V before running for ˜5 h at 120 V for 15% gels and ˜6.5 h for 17% gels. Running buffer (25 mM Tris base, 200 mM glycine, 0.1% (w/v) SDS) was changed every 2 h. The resulting gel was blotted onto PVDF (EMD Millipore 0.45 μm PVDF-FL) using wet transfer in cold transfer buffer (20% (v/v) MeOH, 50 mM Tris base, 400 mM glycine, 0.0373% (w/v) SDS) for 1 h at 90 V. The membrane was blocked using 5% non-fat milk solution in PBS-T (PBS pH 7.4, 0.01% (v/v) Tween-20) over night at 4° C. with gentle agitation. Primary antibodies (rabbit α-Nterm-GFP Sigma Aldrich #G1544) were applied in PBS-T (1:3,000) for 1 h (RT; gentle agitation). The blot was washed in PBS-T (5 min) before secondary antibodies (goat α-rabbit-Alexa Fluor 647-conjugated antibody, ThermoFisher #A32733) were applied in PBS-T (1:20,000) for 45 min (RT; gentle agitation). The blot was washed with PBS-T before (3×5 min) imaging using a Typhoon 9410 laser scanner (Typhoon Scanner Control v5 GE Healthcare Life Sciences) at 50-100 μm resolution, scanning first for AlexaFluor 647 (Ex. 633 nm; Em. 670/30 nm; PMT 500 V) and then TAMRA (Ex. 532 nm; Em. 580/30 nm; PMT 400 V).

### Dual Bioconjugation of PrK-pAzF Labeled Protein

Cell pellets from 1 mL of culture were lyzed using BugBuster (100 μl; EMD Millipore; 15 min at RT; 220 r.p.m.). The lysate was diluted in Buffer W (600 μl) and MagStrep beads were added (200 μl) and allowed to bind (30 min; 4° C.; gentle rotation). The beads were pulled down using a magnet and washed with cold Buffer W (2×1000 μl) before being suspended in Buffer W (200 μl). SPAAC was carried out using half of this suspension with TAMRA-PEG₄-DBCO (0.5 mM) 12-16 h (RT; gently rotation). The beads were washed with EDTA-free Buffer W (2×500 μl; HEPES 50 mM pH 7.4, 150 mM NaCl) before being suspended in EDTA-free Buffer W (100 μl). CuAAC was carried out (1.5 h; RT; gentle rotation) using half of this suspension with Azido-PEG4-TAMRA (0.2 mM) as well as copper(II) sulphate (0.5 mM), tris(benzyltriazolylmethyl)amine (2 mM; THPTA), and sodium ascorbate (15 mM). Beads were washed with Buffer W (2×500 μl) before elutions were done using buffer BXT (10 min; RT; occasional vortexing).

### Intact Protein High-Resolution Mass Spectrometry

Purified protein (5 ug) was desalted into HPLC grade water (4×500 μl) by four cycles of centrifugation through 10K Amicon Ultra Centrifugal filters (EMD Millipore) at 14,000×g (3×10 min and then 1×18 min) as described before. After recovering the protein, 6 μl protein was injected into a Waters I-Class LC connected to a Waters G2-XS TOF. Flow conditions were 0.4 mL/min of 50:50 water:acetonitrile plus 0.1% formic acid. Ionization was done by ESI+ and data was collected for m/z 500-2000. A spectral combine was performed over the main portion of the mass peak and the combined spectrum was deconvoluted using Waters MaxEntl. Analysis was carried out by automated peak integration as well as manual peak identification (FIG. 15, FIG. 16). Fidelity was calculated as the integral of expected mass relative to integrals of all masses identified to be either product or impurity without taking technical impurities into consideration (e.g. salt adducts, arginine oxidation).

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of synthesizing an unnatural polypeptide comprising:
a. providing at least one unnatural deoxyribonucleic acid (DNA) molecule comprising at least four unnatural base pairs, wherein the at least one unnatural DNA molecule encodes (i) a messenger ribonucleic acid (mRNA) molecule comprising at least first and second unnatural codons and (ii) at least first and second transfer RNA (tRNA) molecules, the first tRNA molecule comprising a first unnatural anticodon and the second tRNA molecule comprising a second unnatural anticodon, and the at least four unnatural base pairs in the at least one DNA molecule are in sequence contexts such that the first and second unnatural codons of the mRNA molecule are complementary to the first and second unnatural anticodons, respectively;
b. transcribing the at least one unnatural DNA molecule to afford the mRNA;
c. transcribing the at least one unnatural DNA molecule to afford the at least first and second tRNA molecules; and
d. synthesizing the unnatural polypeptide by translating the unnatural mRNA molecule utilizing the at least first and second unnatural tRNA molecules, wherein each of the at least first and second unnatural anticodons direct site-specific incorporation of an unnatural amino acid into the unnatural polypeptide.

2. The method of claim 1, wherein the at least two unnatural codons each comprise a first unnatural nucleotide positioned at a first position, a second position, or a third position of the codon, optionally wherein the first unnatural nucleotide is positioned at a second position or a third position of the codon.

3. The method of any one of the preceding claims, wherein the at least two unnatural codons each comprises a nucleic acid sequence NNX or NXN, and the unnatural anticodon comprises a nucleic acid sequence XNN, YNN, NXN, or NYN, to form the unnatural codon-anticodon pair comprising NNX-XNN, NNX-YNN, or NXN-NYN, wherein N is any natural nucleotide, X is a first unnatural nucleotide, and Y is a second unnatural nucleotide different from the first unnatural nucleotide, with X-Y forming the unnatural base pair in DNA.

4. The method of claim 3, wherein the codon comprises at least one G or C and the anticodon comprises at least one complementary C or G.

5. The method of claim 3 or 4, wherein X and Y are independently selected from the group consisting of
(i) 2-thiouracil, 2′-deoxyuridine, 4-thio-uracil, uracil-5-yl, hypoxanthin-9-yl (I), 5-halouracil; 5-propynyl-uracil, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil;
(ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1,4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one);
(iii) 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine;
(iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and
(v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

6. The method of claim 4 or 5, wherein the bases comprising each of X and Y are independently selected from the group consisting of:

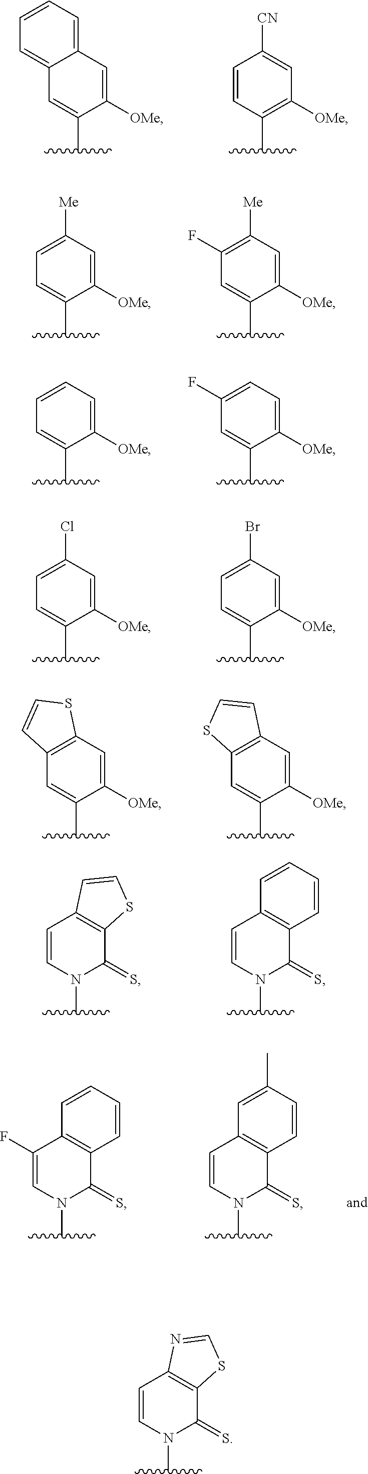

7. The method of claim 6, wherein the base comprising each X is

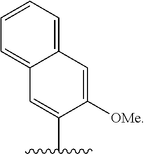 [SMILES: *c1cc2ccccc2cc1OC]

8. The method of claim 6 or 7, wherein the base comprising each Y is

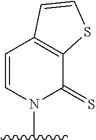 [SMILES: *n1ccc2ccsc2c1=S]

9. The method of any one of claims 3-8, wherein NNX-XNN is selected from the group consisting of UUX-XAA, UGX-XCA, CGX-XCG, AGX-XCU, GAX-XUC, CAX-XUG, AUX-XAU, CUX-XAG, GUX-XAC, UAX-XUA, and GGX-XCC.

10. The method of any one of claims 3-8, wherein NNX-YNN is selected from the group consisting of UUX-YAA, UGX-YCA, CGX-YCG, AGX-YCU, GAX-YUC, CAX-YUG, AUX-YAU, CUX-YAG, GUX-YAC, UAX-YUA, and GGX-YCC.

11. The method of any one of claims 3-8, wherein NXN-NYN is selected from the group consisting of GXU-AYC, CXU-AYG, GXG-CYC, AXG-CYU, GXC-GYC, AXC-GYU, GXA-UYC, CXC-GYG, and UXC-GYA.

12. The method of any one of the preceding claims, wherein the at least two unnatural tRNA molecules each comprise a different unnatural anticodon.

13. The method of claim 12, wherein the at least two unnatural tRNA molecules comprise a pyrrolysyl tRNA from the Methanosarcina genus and the tyrosyl tRNA from Methanocaldococcus jannaschii, or derivatives thereof.

14. The method of any one of claims 11-13, comprising charging the at least two unnatural tRNA molecules by an amino-acyl tRNA synthetase.

15. The method of claim 14, wherein the tRNA synthetase is selected from a group consisting of chimeric PylRS (chPylRS) and M. jannaschii AzFRS (MjpAzFRS).

16. The method of claim 12 or 13, comprising charging the at least two unnatural tRNA molecules by at least two different tRNA synthetases.

17. The method of claim 16, wherein the at least two different tRNA synthetases comprise chimeric PylRS (chPylRS) and M. jannaschii AzFRS (MjpAzFRS).

18. The method of any one of claims 1-17, wherein the unnatural polypeptide comprises two, three, or more unnatural amino acids.

19. The method of any one of claims 1-18, wherein the unnatural polypeptide comprises at least two unnatural amino acids that are the same.

20. The method of any one of claims 1-18, wherein the unnatural polypeptide comprises at least two different unnatural amino acids.

21. The method of any one of claims 1-20, wherein the unnatural amino acid comprises
a lysine analogue;
an aromatic side chain;
an azido group;
an alkyne group; or
an aldehyde or ketone group.

22. The method of any one of the claims 1-20, wherein the unnatural amino acid does not comprise an aromatic side chain.

23. The method of any one of claims 1-20, wherein the unnatural amino acid is selected from N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

24. The method of any one of the preceding claims, wherein the at least one unnatural DNA molecule is in the form of a plasmid.

25. The method of any one of claims 1-23, wherein the at least one unnatural DNA molecule is integrated into the genome of a cell.

26. The method of claim 24 or 25, wherein the at least one unnatural DNA molecule encodes the unnatural polypeptide.

27. The method of any one of the preceding claims, wherein the method comprises the in vivo replication and transcription of the unnatural DNA molecule and the in vivo translation of the transcribed mRNA molecule in a cellular organism.

28. The method of claim 27, wherein the cellular organism is a microorganism.

29. The method of claim 28, wherein the cellular organism is a prokaryote.

30. The method of claim 29, wherein the cellular organism is a bacterium.

31. The method of claim 30, wherein the cellular organism is a gram-positive bacterium.

32. The method of claim 30, wherein the cellular organism is a gram-negative bacterium.

33. The method of claim 32, wherein the cellular organism is Escherichia coli.

34. The method of any one of the preceding claims, wherein the at least two unnatural base pairs comprise base pairs selected from dCNMO-dTPT3, dNaM-dTPT3, dCNMO-dTAT1, or dNaM-dTAT1.

35. The method of any one of claims 27-34, wherein the cellular organism comprises a nucleoside triphosphate transporter.

36. The method of claim 35, wherein the nucleoside triphosphate transporter comprises the amino acid sequence of PtNTT2.

37. The method of claim 36, wherein the nucleoside triphosphate transporter comprises a truncated amino acid sequence of PtNTT2, optionally wherein the truncated amino acid sequence of PtNTT2 is at least 80% identical to a PtNTT2 encoded by SEQ ID NO.1.

38. The method of any one of claims 27-37, wherein the cellular organism comprises the at least one unnatural DNA molecule.

39. The method of claim 38, wherein the at least one unnatural DNA molecule comprises at least one plasmid.

40. The method of claim 38, wherein the at least one unnatural DNA molecule is integrated into the genome of the cell.

41. The method of claim 39 or 40, wherein the at least one unnatural DNA molecule encodes the unnatural polypeptide.

42. The method of any one of claims 1-24, wherein the method is an in vitro method, comprising synthesizing the unnatural polypeptide with a cell-free system.

43. The method of any one of the preceding claims, wherein the unnatural base pairs comprise at least one unnatural nucleotide comprising an unnatural sugar moiety.

44. The method of claim 43, wherein the unnatural sugar moiety comprises a moiety selected from the group consisting of:
a modification at the 2′ position comprising:
OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, or NH₂F;
O-alkyl, S-alkyl, or N-alkyl;
O-alkenyl, S-alkenyl, or N-alkenyl;
O-alkynyl, S-alkynyl, or N-alkynyl;
O-alkyl-O-alkyl, 2′-F, 2′-OCH₃, or 2′-O(CH₂)₂OCH₃, wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —O(CH₂)ₙCH₃, —O(CH₂)ₙ—NH₂, or —O(CH₂)ₙON[(CH₂)ₙCH₃)]₂, wherein n and m are from 1 to about 10;
a modification at the 5′ position comprising:
5′-vinyl, or 5′-methyl (R or S); or
a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide; or
any combination thereof.

45. A cell comprising at least one unnatural DNA molecule comprising at least four unnatural base pairs, wherein the at least one unnatural DNA molecule encodes (i) a messenger ribonucleic acid (mRNA) molecule encoding an unnatural polypeptide and comprising at least first and second unnatural codons; and (ii) at least first and second transfer RNA (tRNA) molecules, the first tRNA molecule comprising a first unnatural anticodon and the second tRNA molecule comprising a second unnatural anticodon, wherein the at least four unnatural base pairs in the at least one DNA molecule are in sequence contexts such that the first and second unnatural codons of the mRNA molecule are complementary to the first and second unnatural anticodons, respectively.

46. The cell of claim 45, further comprising the mRNA molecule and the at least first and second tRNA molecules.

47. The cell of claim 46, wherein the at least first and second tRNA molecules are covalently linked to unnatural amino acids.

48. The cell of claim 47, further comprising the unnatural polypeptide.

49. A cell comprising:
a. at least two different unnatural codon-anticodon pairs, wherein each unnatural codon-anticodon pair comprises an unnatural codon from an unnatural messenger RNA (mRNA) and an unnatural anticodon from an unnatural transfer ribonucleic acid (tRNA), said unnatural codon comprising a first unnatural nucleotide and said unnatural anticodon comprising a second unnatural nucleotide; and
b. at least two different unnatural amino acids each covalently linked to a corresponding unnatural tRNA.

50. The cell of claim 49, further comprising at least one unnatural DNA molecule comprising at least four unnatural base pairs (UBPs).

51. The cell of any one of claims 45-50, wherein the first unnatural nucleotide is positioned at a second or a third position of the unnatural codon.

52. The cell of claim 51, wherein the first unnatural nucleotide is complementarily base paired with the second unnatural nucleotide of the unnatural anticodon.

53. The cell of any one of claims 45-52, wherein the first unnatural nucleotide and the second unnatural nucleotide comprise first and second bases, respectively, independently selected from the group consisting of 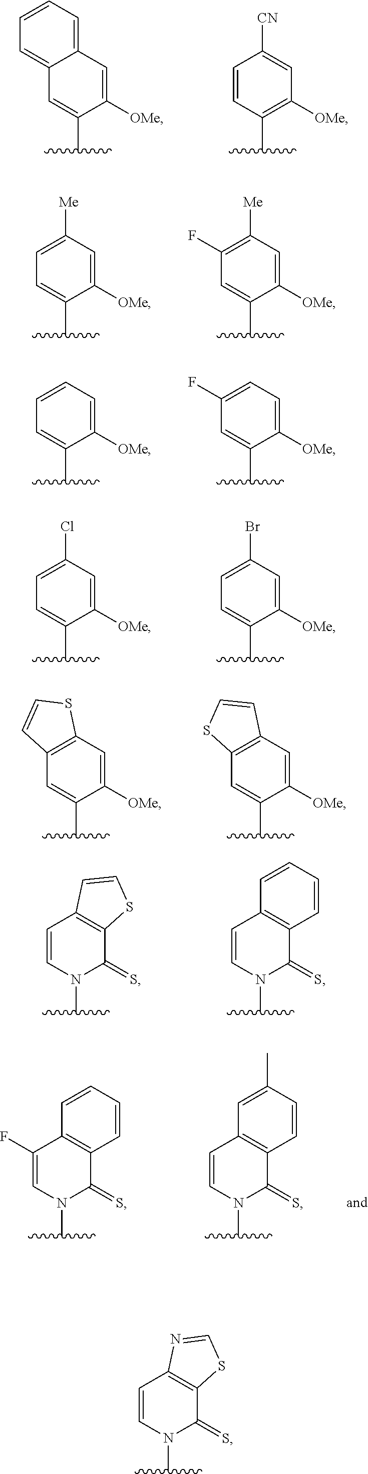
wherein the second base is different from the first base.

54. The cell of any one of claim 45 or 47-53, wherein the at least four unnatural base pairs are independently selected from the group consisting of dCNMO/dTPT3, dNaM/dTPT3, dCNMO/dTAT1, or dNaM/dTAT1.

55. The cell of any one of claim 45 or 47-54, wherein the at least one unnatural DNA molecule comprises at least one plasmid.

56. The cell of any one of claim 45 or 47-54, wherein the at least one unnatural DNA molecule is integrated into genome of the cell.

57. The cell of any one of claims 47-56, wherein the at least one unnatural DNA molecule encodes an unnatural polypeptide.

58. The cell of any one of claims 45-57, wherein the cell expresses a nucleoside triphosphate transporter.

59. The cell of claim 58, wherein the nucleoside triphosphate transporter comprises the amino acid sequence of PtNTT2.

60. The method of claim 59, wherein the nucleoside triphosphate transporter comprises a truncated amino acid sequence of PtNTT2, optionally wherein the truncated amino acid sequence of PtNTT2 is at least 80% identical to a PtNTT2 encoded by SEQ ID NO.1.

61. The cell of any one of claims 45 to 60, wherein the cell expresses at least two tRNA synthetases.

62. The cell of claim 61, wherein the at least two tRNA synthetases are chimeric PylRS (chPylRS) and M. jannaschii AzFRS (MjpAzFRS).

63. The cell of any one of claims 45 to 62, wherein the cell comprises unnatural nucleotides comprising an unnatural sugar moiety.

64. The cell of claim 63, wherein the unnatural sugar moiety is selected from the group consisting of:
a modification at the 2′ position comprising
OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, or NH₂F;
O-alkyl, S-alkyl, or N-alkyl;
O-alkenyl, S-alkenyl, or N-alkenyl;
O-alkynyl, S-alkynyl, or N-alkynyl;
O-alkyl-O-alkyl, 2′-F, 2′-OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —O(CH₂)ₙCH₃, —O(CH₂)ₙ—NH₂, or —O(CH₂)ₙON[(CH₂)ₙCH₃)]₂, wherein n and m are from 1 to about 10;
a modification at the 5′ position comprising:
5′-vinyl, 5′-methyl (R or S); or
a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide; or
any combination thereof.

65. The cell of any one of claims 45 to 64, wherein at least one unnatural nucleotide base is recognized by an RNA polymerase during transcription.

66. The cell of any one of claims 45 to 65, wherein the cell translates at least one unnatural polypeptide comprising the at least two unnatural amino acids.

67. The cell of any one of claim 45 to 66, wherein the at least two unnatural amino acids are independently selected from the group consisting of N6-azidoethoxy-carbonyl-L-lysine (AzK), N6-propargylethoxy-carbonyl-L-lysine (PraK), N6-(propargyloxy)-carbonyl-L-lysine (PrK), p-azido-phenylalanine(pAzF), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

68. The cell of any one of claims 45 to 67, wherein the cell is isolated.

69. The cell of any one of claims 45 to 68, wherein the cell is a prokaryote.

70. A cell line comprising the cell of any one of claims 45 to 69.
